(19) United States
(12) Patent Application Publication
(10) Pub. No.: US 2017/0002421 A1
(43) Pub. Date: Jan. 5, 2017

(54) METHODS AND ASSAYS FOR DETERMINING REDUCED BRCA1 PATHWAY FUNCTION IN A CANCER CELL

(71) Applicant: THE GENERAL HOSPITAL CORPORATION, Boston, MA (US)

(72) Inventors: Dennis Sgroi, Winchester, MA (US); Ryan P. McMullin, Princeton, MA (US); Ben Wittner, Salem, MA (US); Sridhar Ramaswamy, Wellesley, MA (US)

(73) Assignee: THE GENERAL HOSPITAL CORPORATION, Boston, MA (US)

(21) Appl. No.: 15/107,176

(22) PCT Filed: Dec. 22, 2014
(86) PCT No.: PCT/US2014/071948

§ 371 Date: Jun. 22, 2016

Related U.S. Application Data
(60) Provisional application No. 61/919,890, filed on Dec. 23, 2013.

(51) Int. Cl. C12Q 1/68

(52) U.S. Cl. CPC C12Q 1/6886; C12Q 2600/158; C12Q 2600/106

(57) ABSTRACT

Provided herein are methods and assays that can be used to predict whether a subject will or will not respond to a cancer treatment comprising a PARP inhibitor and a DNA damage-inducing chemotherapeutic.

### CROSS-REFERENCE TO RELATED APPLICATIONS

This application claims benefit under 35 U.S.C. §119(e) of U.S. Provisional Application Ser. No. 61/919,890 filed on Dec. 23, 2013, the contents of which are herein incorporated by reference in its entirety.

### TECHNICAL FIELD

The invention relates to the diagnosis of a cancer subtype and for predicting sensitivity of such cancers to combination treatment with a PARP inhibitor and a DNA damage inducing chemotherapeutic.

### BACKGROUND

Central nervous system metastases are diagnosed in approximately 10% to 16% of women with advanced breast cancer^{1,2}. The total incidence of brain metastases is potentially higher than currently reported statistics, as most brain metastases are diagnosed in response to clinical symptoms rather than by an initial detection. Several risk factors have been associated with the development of brain lesions in patients with metastatic breast cancer (MBC), including a younger age³, having more than two metastatic sites at diagnosis³, negative estrogen receptor (ER) status^{1,4,5}, HER2+ disease^{1,4}, and BRCA1/2 mutation⁶⁻⁸. Survival for breast cancer patients with brain metastases is poor, with a 1-year survival probability of approximately 20%². Patients with HER2+ MBC tumors are 2 to 4 times more likely to develop brain metastases than patients with HER2-negative disease^{1,4}. While systemic Trastuzumab has proven efficacious for treating aggressive HER2+ breast cancer, its use has been associated with the central nervous system as the first site of relapse⁹. It would be useful to develop methods to predict which cancer patients have a better chance of responding to treatments to allow more targeted treatment and reduce to exposure to side effects from treatments that can be predicted not to work with certain types of cancers.

### SUMMARY

The methods and assays described herein are based, in part, on the discovery that the expression level of a combination of markers, typically when normalized, can be used to predict sensitivity of a cancer to a combination treatment comprising a poly ADP ribose polymerase (PARP) inhibitor and a DNA damage inducing chemotherapeutic (e.g., a DNA alkylating agent). Provided herein are methods, assays and compositions that take advantage of this discovery to permit the selection of a treatment for a cancer, classifying a cancer subject into responsive and non-responsive subgroups, and determining reduced function of the BRCA1 pathway in a cancer cell to allow prediction of the response of the cell to certain types of cancer treatments.

Provided herein in one aspect is a method of treating cancer in a subject, the method comprising: (a) assaying a biological sample comprising a cancer cell obtained from a subject having cancer or suspected of having cancer for the expression of a combination of at least the following markers: EZR, SOX4, EMG1, NARS, SNRNP70, CLTA, EIF3B, EIF3E, UBAP2L, SF3A3, HK1, CKAP4, NOLC1, HCFC1, PPM1F, BMS1, AIMP2, POLG, MCM2, U2AF1, MOGS, SKIV2L, CCND1, MLEC, RARS, SLC7A5, MANF, RAE1, CHD4, BYSL, GTF3C2, TRAP1, KIAA0114, CDC25B, SF3B2, SMARCA4, DDX11, LMNB2, DHCR24, SAFB, CTPS, DUSP1, DDX10, CDC123, NDRG1, EIF2B2, FAM38A, SEC24C, RABEPK, FGFR3, SSRP1 and SLC1A3; (b) normalizing the expression data for each of the markers to a control, and (c) administering to the subject a therapeutically effective amount of a poly ADP ribose polymerase (PARP) inhibitor and a DNA damage-inducing chemotherapeutic agent when expression of the combination of markers is increased compared to a reference value and not administering a PARP inhibitor and a DNA damage-inducing chemotherapeutic agent when the expression of the combination of markers is not changed or decreased compared to a reference value.

Also provided herein in another aspect is a method of determining a BRCA1 functional pathway-deficient cancer in a subject, the method comprising: (a) assaying a biological sample comprising a cancer cell obtained from a subject having cancer or suspected of having cancer for the expression of a combination of at least the following markers: EZR, SOX4, EMG1, NARS, SNRNP70, CLTA, EIF3B, EIF3E, UBAP2L, SF3A3, HK1, CKAP4, NOLC1, HCFC1, PPM1F, BMS1, AIMP2, POLG, MCM2, U2AF1, MOGS, SKIV2L, CCND1, MLEC, RARS, SLC7A5, MANF, RAE1, CHD4, BYSL, GTF3C2, TRAP1, KIAA0114, CDC25B, SF3B2, SMARCA4, DDX11, LMNB2, DHCR24, SAFB, CTPS, DUSP1, DDX10, CDC123, NDRG1, EIF2B2, FAM38A, SEC24C, RABEPK, FGFR3, SSRP1, and SLC1A3; and wherein if expression of the combination of markers is increased compared to a reference value the subject is determined to have a BRCA1 functional pathway deficient cancer, and wherein if the expression of the combination of markers is not changed or reduced compared to the reference value, the subject is determined not to have a BRCA1 functional pathway deficient cancer.

In one embodiment of this aspect and all other aspects described herein, the method further comprises assaying the biological sample for at least one additional marker selected from the group consisting of: GCSH, BOP1, XRCC5, CCDC85B, H1FX, FDFT1, DNAJC7, PRKACA, MAP2K5, SNRPA1, RELA, RXRA, KDM5C, PWP2, NME3, FARSA, DNTTIP2, MRPS12, SCRIB, PPAT, CDC37, SLC29A1, SFRS11, SMAD6, EIF4H, VDAC1, NMT1, POLRMT, POLD1, CHAF1A, TAF1C, TCOF1, ALG3, CAD, RASSF7, MTHFD1, CCNE1, HSF1, PSMD1, AGPAT2, PHKG2, PKN1, GAL, SCAP, EIF4G1, DDT, PMPCA, RRP1B, FKBP4, NUP214, ATIC, FASN, AP3D1, RPIA, NSDHL, BRD2, PDCD11, SON, HSP90B1, and ANXA11.

In some aspects, of all the embodiments, assaying is performed using at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, or at least 50 of the genes selected from the group consisting of: EZR, SOX4, EMG1, NARS, SNRNP70, CLTA, EIF3B, EIF3E, UBAP2L, SF3A3, HK1, CKAP4, NOLC1, HCFC1, PPM1F, BMS1, AIMP2, POLG, MCM2, U2AF1, MOGS, SKIV2L, CCND1, MLEC, RARS, SLC7A5, MANF, RAE1, CHD4, BYSL, GTF3C2, TRAP1, KIAA0114, CDC25B, SF3B2, SMARCA4, DDX11, LMNB2, DHCR24, SAFB, CTPS, DUSP1, DDX10, CDC123, NDRG1, EIF2B2, FAM38A, SEC24C, RABEPK, FGFR3, SSRP1, and SLC1A3.

In some aspects, of all the embodiments, assaying is performed using at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, or at least 50, at least 51, at least 52, at least 53, at least 54, at least 55, at least 56, at least 57, at least 58, or at least 59 of the genes selected from the group consisting of: GCSH, BOP1, XRCC5, CCDC85B, H1FX, FDFT1, DNAJC7, PRKACA, MAP2K5, SNRPA1, RELA, RXRA, KDM5C, PWP2, NME3, FARSA, DNTTIP2, MRPS12, SCRIB, PPAT, CDC37, SLC29A1, SFRS11, SMAD6, EIF4H, VDAC1, NMT1, POLRMT, POLD1, CHAF1A, TAF1C, TCOF1, ALG3, CAD, RASSF7, MTHFD1, CCNE1, HSF1, PSMD1, AGPAT2, PHKG2, PKN1, GAL, SCAP, EIF4G1, DDT, PMPCA, RRP1B, FKBP4, NUP214, ATIC, FASN, AP3D1, RPIA, NSDHL, BRD2, PDCD11, SON, HSP90B1, and ANXA11.

In another embodiment of this aspect and all other aspects described herein, the control is the expression level of the total set of markers assayed.

In another embodiment of this aspect and all other aspects described herein, the method further comprises a step of calculating a metagene value to represent the normalized expression of the combination of markers.

In another embodiment of this aspect and all other aspects described herein, the step of calculating the metagene comprises linear combination of the expression values of each marker in the combination of markers.

In another embodiment of this aspect and all other aspects described herein, the linear combination has a coefficient of 1 for each marker.

In another embodiment of this aspect and all other aspects described herein, the biological sample comprises a tumor sample.

In another embodiment of this aspect and all other aspects described herein, the biological sample is assayed for expression of all of the markers SFRS11, EZR, PKN1, SOX4, EMG1, RELA, AIMP2, NARS, HSP90B1, SNRP70, NME3, CLTA, CAD, EIF3B, EIF3E, UBAP2L, SF3A3, HK1, TCOF1, CKAP4, CCNE1, NOLC1, HCFC1, PPM1F, BMS1L, RASSF7, FDFT1, FARSLA, POLG, MCM2, VDAC1, U2AF1, MOGS, ATIC, CHAF1A, SKIV2L, GCSH, GAL, CCDC85B, CCND1, MLEC, RARS, WBSCR1, FKBP4, SLC7A5, MTHFD1, NSDHL, PWP2H, FASN, MANF, EIF4G1, JARID1C, SCRIB, SCAP, PSMD1, RAE1, CHD4, SLC29A1, BYSL, GTF3C2, TRAP1, PRKACA, KIAA0179, KIAA0114, POLD1, DNTTIP2, BOP1, CDC25B, MRPS12, SF3B2, NUP214, PHKG2, AGPAT2, SMARCA4, PPAT, SNRPA1, DDX11, RPIA, ALG3, LMNB2, TAF1C, DDT, DHCR24, SAFB, CDC37, H1FX, AP3D1, DNAJC7, HSF1, CTPS, DUSP1, DDX10, PDCD11, CDC123, RXRA, NDRG1, EIF2B2, ANXA11, FAM38A, XRCC5, SEC24C, PMPCA, POLRMT, RABEPK, SMAD6, NMT1, SON, MAP2K5, SLC1A3, FGFR3, SSRP1 and BRD2.

In another embodiment of this aspect and all other aspects described herein, the subject is known to carry a mutation in the BRCA1 gene.

In another embodiment of this aspect and all other aspects described herein, the subject is known to carry a wild-type BRCA1 gene.

In another embodiment of this aspect and all other aspects described herein, the BRCA1 status of the subject is unknown.

In another embodiment of this aspect and all other aspects described herein, the cancer is breast cancer.

In another embodiment of this aspect and all other aspects described herein, the expression assayed is mRNA expression.

In another embodiment of this aspect and all other aspects described herein, the expression assayed is protein expression.

Also provided herein, in another aspect, is a method for selecting a PARP inhibitor/DNA damaging chemotherapeutic-sensitive or PARP inhibitor/DNA damaging chemotherapeutic-insensitive subject having cancer, the method comprising: (a) assaying a biological sample comprising a cancer cell obtained from a subject having cancer or suspected of having cancer for the expression of a combination of at least the following markers: EZR, SOX4, EMG1, NARS, SNRNP70, CLTA, EIF3B, EIF3E, UBAP2L, SF3A3, HK1, CKAP4, NOLC1, HCFC1, PPM1F, BMS1, AIMP2, POLG, MCM2, U2AF1, MOGS, SKIV2L, CCND1, MLEC, RARS, SLC7A5, MANF, RAE1, CHD4, BYSL, GTF3C2, TRAP1, KIAA0114, CDC25B, SF3B2, SMARCA4, DDX11, LMNB2, DHCR24, SAFB, CTPS, DUSP1, DDX10, CDC123, NDRG1, EIF2B2, FAM38A, SEC24C, RABEPK, FGFR3, SSRP1, and SLC1A3; (b) normalizing the expression data for each of the markers to a control, and (c) selecting the subject as a PARP inhibitor/DNA damaging chemotherapeutic-sensitive subject when expression of the combination of markers is increased compared to a reference value, and selecting the subject as a PARP inhibitor/DNA damaging chemotherapeutic-insensitive subject when expression of the combination of markers is not changed or decreased compared to the reference value.

In one embodiment of this aspect and all other aspects described herein, the method further comprises assaying the biological sample for at least one additional marker selected from the group consisting of: GCSH, BOP1, XRCC5, CCDC85B, H1FX, FDFT1, DNAJC7, PRKACA, MAP2K5, SNRPA1, RELA, RXRA, KDM5C, PWP2, NME3, FARSA, DNTTIP2, MRPS12, SCRIB, PPAT, CDC37, SLC29A1, SFRS11, SMAD6, EIF4H, VDAC1, NMT1, POLRMT, POLD1, CHAF1A, TAF1C, TCOF1, ALG3, CAD, RASSF7, MTHFD1, CCNE1, HSF1, PSMD1, AGPAT2, PHKG2, PKN1, GAL, SCAP, EIF4G1, DDT, PMPCA, RRP1B, FKBP4, NUP214, ATIC, FASN, AP3D1, RPIA, NSDHL, BRD2, PDCD11, SON, HSP90B1, and ANXA11.

In another embodiment of this aspect and all other aspects described herein, the biological sample is assayed for expression of all of the markers SFRS11, EZR, PKN1, SOX4, EMG1, RELA, AIMP2, NARS, HSP90B1, SNRP70, NME3, CLTA, CAD, EIF3B, EIF3E, UBAP2L, SF3A3, HK1, TCOF1, CKAP4, CCNE1, NOLC1, HCFC1, PPM1F, BMS1L, RASSF7, FDFT1, FARSLA, POLG, MCM2, VDAC1, U2AF1, MOGS, ATIC, CHAF1A, SKIV2L, GCSH, GAL, CCDC85B, CCND1, MLEC, RARS, WBSCR1, FKBP4, SLC7A5, MTHFD1, NSDHL, PWP2H, FASN, MANF, EIF4G1, JARID1C, SCRIB, SCAP, PSMD1, RAE1, CHD4, SLC29A1, BYSL, GTF3C2, TRAP1, PRKACA, KIAA0179, KIAA0114, POLD1, DNTTIP2, BOP1, CDC25B, MRPS12, SF3B2, NUP214, PHKG2, AGPAT2, SMARCA4, PPAT, SNRPA1, DDX11, RPIA, ALG3, LMNB2, TAF1C, DDT, DHCR24, SAFB, CDC37, H1FX, AP3D1, DNAJC7, HSF1, CTPS, DUSP1, DDX10, PDCD11, CDC123, RXRA, NDRG1, EIF2B2, ANXA11, FAM38A, XRCC5, SEC24C, PMPCA, POLRMT, RABEPK, SMAD6, NMT1, SON, MAP2K5, SLC1A3, FGFR3, SSRP1, and BRD2.

In another embodiment of this aspect and all other aspects described herein, the control is the expression level of the total set of markers assayed.

In another embodiment of this aspect and all other aspects described herein, the method further comprises a step of calculating a metagene value to represent the normalized expression of the combination of markers.

In another embodiment of this aspect and all other aspects described herein, the step of calculating the metagene comprises linear combination of the expression values of each marker in the combination of markers.

In another embodiment of this aspect and all other aspects described herein, the linear combination has a coefficient of 1 for each marker.

In another embodiment of this aspect and all other aspects described herein, the biological sample comprises a tumor sample.

In another embodiment of this aspect and all other aspects described herein, the subject carries a mutation in the BRCA1 gene.

In another embodiment of this aspect and all other aspects described herein, the subject carries wild-type BRCA1 gene.

In another embodiment of this aspect and all other aspects described herein, the BRCA1 status of the subject is unknown.

In another embodiment of this aspect and all other aspects described herein, the cancer is breast cancer.

In another embodiment of this aspect and all other aspects described herein, the expression assayed is mRNA expression.

In another embodiment of this aspect and all other aspects described herein, the expression assayed is protein expression.

Also provided herein are methods of determining reduced BRCA1 pathway function in a cancer cell, the method comprising: (a) assaying a biological sample comprising a cancer cell for expression of at least the following markers: EZR, SOX4, EMG1, NARS, SNRNP70, CLTA, EIF3B, EIF3E, UBAP2L, SF3A3, HK1, CKAP4, NOLC1, HCFC1, PPM1F, BMS1, AIMP2, POLG, MCM2, U2AF1, MOGS, SKIV2L, CCND1, MLEC, RARS, SLC7A5, MANF, RAE1, CHD4, BYSL, GTF3C2, TRAP1, KIAA0114, CDC25B, SF3B2, SMARCA4, DDX11, LMNB2, DHCR24, SAFB, CTPS, DUSP1, DDX10, CDC123, NDRG1, EIF2B2, FAM38A, SEC24C, RABEPK, FGFR3, SSRP1, and SLC1A3, and (b) normalizing the expression data for each of the markers to a control, wherein the cancer cell is determined to have reduced BRCA1 pathway function when the metagene value is increased compared to a reference value, and wherein the cancer cell is determined not to have reduced BRCA1 pathway function when the metagene value is not changed or reduced compared to a reference value.

In one embodiment of this aspect and all other aspects described herein, the control is the expression level of the total set of markers assayed.

In another embodiment of this aspect and all other aspects described herein, the method further comprises a step of calculating a metagene value to represent the normalized expression of the combination of markers.

In another embodiment of this aspect and all other aspects described herein, the step of calculating the metagene comprises linear combination of the expression values of each marker in the combination of markers.

In another embodiment of this aspect and all other aspects described herein, the linear combination has a coefficient of 1 for each marker.

In another embodiment of this aspect and all other aspects described herein, the biological sample comprises a tumor sample.

In another embodiment of this aspect and all other aspects described herein, the method further comprises assaying the biological sample for at least one additional marker selected from the group consisting of: GCSH, BOP1, XRCC5, CCDC85B, H1FX, FDFT1, DNAJC7, PRKACA, MAP2K5, SNRPA1, RELA, RXRA, KDM5C, PWP2, NME3, FARSA, DNTTIP2, MRPS12, SCRIB, PPAT, CDC37, SLC29A1, SFRS11, SMAD6, EIF4H, VDAC1, NMT1, POLRMT, POLD1, CHAF1A, TAF1C, TCOF1, ALG3, CAD, RASSF7, MTHFD1, CCNE1, HSF1, PSMD1, AGPAT2, PHKG2, PKN1, GAL, SCAP, EIF4G1, DDT, PMPCA, RRP1B, FKBP4, NUP214, ATIC, FASN, AP3D1, RPIA, NSDHL, BRD2, PDCD11, SON, HSP90B1, and ANXA11.

In another embodiment of this aspect and all other aspects described herein, the biological sample is assayed for expression of all of the markers SFRS11, EZR, PKN1, SOX4, EMG1, RELA, AIMP2, NARS, HSP90B1, SNRP70, NME3, CLTA, CAD, EIF3B, EIF3E, UBAP2L, SF3A3, HK1, TCOF1, CKAP4, CCNE1, NOLC1, HCFC1, PPM1F, BMS1L, RASSF7, FDFT1, FARSLA, POLG, MCM2, VDAC1, U2AF1, MOGS, ATIC, CHAF1A, SKIV2L, GCSH, GAL, CCDC85B, CCND1, MLEC, RARS, WBSCR1, FKBP4, SLC7A5, MTHFD1, NSDHL, PWP2H, FASN, MANF, EIF4G1, JARID1C, SCRIB, SCAP, PSMD1, RAE1, CHD4, SLC29A1, BYSL, GTF3C2, TRAP1, PRKACA, KIAA0179, KIAA0114, POLD1, DNTTIP2, BOP1, CDC25B, MRPS12, SF3B2, NUP214, PHKG2, AGPAT2, SMARCA4, PPAT, SNRPA1, DDX11, RPIA, ALG3, LMNB2, TAF1C, DDT, DHCR24, SAFB, CDC37, H1FX, AP3D1, DNAJC7, HSF1, CTPS, DUSP1, DDX10, PDCD11, CDC123, RXRA, NDRG1, EIF2B2, ANXA11, FAM38A, XRCC5, SEC24C, PMPCA, POLRMT, RABEPK, SMAD6, NMT1, SON, MAP2K5, SLC1A3, FGFR3, SSRP1, and BRD2.

In another embodiment of this aspect and all other aspects described herein, the subject is known to carry a mutation in the BRCA1 gene.

In another embodiment of this aspect and all other aspects described herein, the subject is known to carry a wild-type BRCA1 gene.

In another embodiment of this aspect and all other aspects described herein, the BRCA1 status of the subject is unknown.

In another embodiment of this aspect and all other aspects described herein, the cancer is breast cancer.

In another embodiment of this aspect and all other aspects described herein, the expression assayed is mRNA expression.

In another embodiment of this aspect and all other aspects described herein, the expression assayed is protein expression.

In another embodiment of this aspect and all other aspects described herein, the PARP inhibitor is olaparib.

In another embodiment of this aspect and all other aspects described herein, the DNA damage inducing chemotherapeutic agent is temozolomide.

Another aspect provided herein relates to a composition comprising an array consisting essentially of probes directed to the following genes: EZR, SOX4, EMG1, NARS, SNRNP70, CLTA, EIF3B, EIF3E, UBAP2L, SF3A3, HK1, CKAP4, NOLC1, HCFC1, PPM1F, BMS1, AIMP2, POLG, MCM2, U2AF1, MOGS, SKIV2L, CCND1, MLEC, RARS, SLC7A5, MANF, RAE1, CHD4, BYSL, GTF3C2, TRAP1, KIAA0114, CDC25B, SF3B2, SMARCA4, DDX11, LMNB2, DHCR24, SAFB, CTPS, DUSP1, DDX10, CDC123, NDRG1, EIF2B2, FAM38A, SEC24C, RABEPK, FGFR3, SSRP1, and SLC1A3; and attached to a solid support.

In one embodiment of this aspect and all other aspects described herein, the probes are cDNA probes.

Another aspect provided herein relates to a composition comprising an array consisting essentially of probes directed to the following genes: SFRS11, EZR, PKN1, SOX4, EMG1, RELA, AIMP2, NARS, HSP90B1, SNRP70, NME3, CLTA, CAD, EIF3B, EIF3E, UBAP2L, SF3A3, HK1, TCOF1, CKAP4, CCNE1, NOLC1, HCFC1, PPM1F, BMS1L, RASSF7, FDFT1, FARSLA, POLG, MCM2, VDAC1, U2AF1, MOGS, ATIC, CHAF1A, SKIV2L, GCSH, GAL, CCDC85B, CCND1, MLEC, RARS, WBSCR1, FKBP4, SLC7A5, MTHFD1, NSDHL, PWP2H, FASN, MANF, EIF4G1, JARID1C, SCRIB, SCAP, PSMD1, RAE1, CHD4, SLC29A1, BYSL, GTF3C2, TRAP1, PRKACA, KIAA0179, KIAA0114, POLD1, DNTTIP2, BOP1, CDC25B, MRPS12, SF3B2, NUP214, PHKG2, AGPAT2, SMARCA4, PPAT, SNRPA1, DDX11, RPIA, ALG3, LMNB2, TAF1C, DDT, DHCR24, SAFB, CDC37, H1FX, AP3D1, DNAJC7, HSF1, CTPS, DUSP1, DDX10, PDCD11, CDC123, RXRA, NDRG1, EIF2B2, ANXA11, FAM38A, XRCC5, SEC24C, PMPCA, POLRMT, RABEPK, SMAD6, NMT1, SON, MAP2K5, SLC1A3, FGFR3, SSRP1, and BRD2.

In one embodiment of this aspect and all other aspects described herein, the probes are cDNA probes.

Also provided herein, in another aspect, is a method of treating cancer in a subject, the method comprising: administering to a subject a therapeutically effective amount of a poly ADP ribose polymerase (PARP) inhibitor and a DNA damage-inducing chemotherapeutic agent when the subject has been determined to have increased expression of a metagene comprising a combination of markers selected from the group consisting of EZR, SOX4, EMG1, NARS, SNRNP70, CLTA, EIF3B, EIF3E, UBAP2L, SF3A3, HK1, CKAP4, NOLC1, HCFC1, PPM1F, BMS1, AIMP2, POLG, MCM2, U2AF1, MOGS, SKIV2L, CCND1, MLEC, RARS, SLC7A5, MANF, RAE1, CHD4, BYSL, GTF3C2, TRAP1, KIAA0114, CDC25B, SF3B2, SMARCA4, DDX11, LMNB2, DHCR24, SAFB, CTPS, DUSP1, DDX10, CDC123, NDRG1, EIF2B2, FAM38A, SEC24C, RABEPK, FGFR3, SSRP1, and SLC1A3 as compared to a reference value, and not administering a PARP inhibitor and a DNA damage-inducing chemotherapeutic agent when the subject has been determined to have unchanged or decreased expression of a metagene comprising a combination of markers selected from the group consisting of EZR, SOX4, EMG1, NARS, SNRNP70, CLTA, EIF3B, EIF3E, UBAP2L, SF3A3, HK1, CKAP4, NOLC1, HCFC1, PPM1F, BMS1, AIMP2, POLG, MCM2, U2AF1, MOGS, SKIV2L, CCND1, MLEC, RARS, SLC7A5, MANF, RAE1, CHD4, BYSL, GTF3C2, TRAP1, KIAA0114, CDC25B, SF3B2, SMARCA4, DDX11, LMNB2, DHCR24, SAFB, CTPS, DUSP1, DDX10, CDC123, NDRG1, EIF2B2, FAM38A, SEC24C, RABEPK, FGFR3, SSRP1, and SLC1A3 as compared to a reference value.

Another aspect provided herein relates to methods of treating cancer in a human in need thereof, the method comprising (a) selecting a human who has increased expression compared to a reference value of the combination of at least five markers selected from the following markers: EZR, SOX4, EMG1, NARS, SNRNP70, CLTA, EIF3B, EIF3E, UBAP2L, SF3A3, HK1, CKAP4, NOLC1, HCFC1, PPM1F, BMS1, AIMP2, POLG, MCM2, U2AF1, MOGS, SKIV2L, CCND1, MLEC, RARS, SLC7A5, MANF, RAE1, CHD4, BYSL, GTF3C2, TRAP1, KIAA0114, CDC25B, SF3B2, SMARCA4, DDX11, LMNB2, DHCR24, SAFB, CTPS, DUSP1, DDX10, CDC123, NDRG1, IF2B2, FAM38A, SEC24C, RABEPK, FGFR3, SSRP1, and SLC1A3 in a biological sample; and (b) administering to said human a therapeutically effective amount of a poly ADP ribose polymerase (PARP) inhibitor and a DNA damage-inducing chemotherapeutic agent.

In one embodiment of this aspect and all other aspects provided herein, the step of selecting comprises assaying a biological sample from the human for a nucleotide sequence encoding the at least five markers.

In another embodiment of this aspect and all other aspects provided herein, the assaying comprises nucleic acid amplification and optionally one or more methods selected from sequencing, next generation sequencing, nucleic acid hybridization, and allele-specific amplification.

In another embodiment of this aspect and all other aspects provided herein, the assaying is performed in a multiplex format.

In another embodiment of this aspect and all other aspects provided herein, the method further comprises obtaining the biological sample from the human.

In another embodiment of this aspect and all other aspects provided herein, the biological sample comprises serum, blood, feces, tissue, a cell, urine and/or saliva of the human.

Another aspect provided herein relates to a method of treating cancer in a human in need thereof, the method comprising (a) selecting a human who has comparable expression compared to a reference value of the combination of at least five markers selected from the following markers: EZR, SOX4, EMG1, NARS, SNRNP70, CLTA, EIF3B, EIF3E, UBAP2L, SF3A3, HK1, CKAP4, NOLC1, HCFC1, PPM1F, BMS1, AIMP2, POLG, MCM2, U2AF1, MOGS, SKIV2L, CCND1, MLEC, RARS, SLC7A5, MANF, RAE1, CHD4, BYSL, GTF3C2, TRAP1, KIAA0114, CDC25B, SF3B2, SMARCA4, DDX11, LMNB2, DHCR24, SAFB, CTPS, DUSP1, DDX10, CDC123, NDRG1, IF2B2, FAM38A, SEC24C, RABEPK, FGFR3, SSRP1, and SLC1A3 in a biological sample; and (b) administering to said human a therapeutically effective amount of a cancer treatment that does not comprise poly ADP ribose polymerase (PARP) inhibitor and a DNA damage-inducing chemotherapeutic agent.

In one embodiment of this aspect and all other aspects provided herein, the step of selecting comprises assaying a biological sample from the human for a nucleotide sequence encoding the at least five markers.

In another embodiment of this aspect and all other aspects provided herein, the assaying comprises nucleic acid amplification and optionally one or more methods selected from sequencing, next generation sequencing, nucleic acid hybridization, and allele-specific amplification.

In another embodiment of this aspect and all other aspects provided herein, the assaying is performed in a multiplex format.

In another embodiment of this aspect and all other aspects provided herein, the method further comprises obtaining the biological sample from the human.

In another embodiment of this aspect and all other aspects provided herein, the biological sample comprises serum, blood, feces, tissue, a cell, urine and/or saliva of said human.

### BRIEF DESCRIPTION OF THE FIGURES

FIGS. 1A-1B show examples of BRCA1-metagene values in primary breast cancer and metastatic breast cancer samples as calculated from a previously described cohort described by Zhang et al. (2009) Cancer Cell 16:67-78. FIG. 1A shows an example dot blot of individual metagene values. FIG. 1B shows an example of a box plot of metagene values. Statistically significant differences in BD-L metagene value were observed for the brain metastases when compared to primary tumors (p-value=0.0043), bone metastases (p-value=4×10⁻⁶), and lung metastases (p-value=0.001).

FIGS. 2A-2B show examples of BRCA1-metagene values in primary sporadic breast cancer and BRCA1 and BRCA2 primary breast cancer samples as calculated from a previously described cohort described by van't Veer et al. (2002) Nature 415:530-6. FIG. 2A shows an example dot plot of individual metagene values. FIG. 2B shows an example box plot of metagene values. A statistically significant difference in BD-L metagene value was observed for sporadic primary breast tumors when compared to mutant BRCA1 carrier primary breast tumors (p-value=0.033).

FIGS. 3A-3C show an example of a dot plot and box plot distribution of BD-L metagene values by ER and HER2 status and correlation with clinical outcome in the NK1295 (FIG. 3A), EMC286/MSK82 (FIG. 3B), and EMC192 (FIG. 3C) cohorts. Statistically significant differences in BD-L metagene values were observed for ER−/HER− primary tumors when compared to ER+/HER2+(p-value=4.5×10⁻⁶, 0.0025, and 2.8×10⁻⁵ in FIGS. 3A, 3B, and 3C, respectively) and ER+/HER2− (p-value=1.1×10⁻¹³, 4.0×10⁻⁸, and 8.7×10⁻¹⁰ in FIGS. 3A, 3B, and 3C, respectively) subgroups.

FIGS. 4A-4D show linear regression analyses of single and combination treatment with 40 nM olaparib and 100 um temozolomide using: BRCA1 deficient-like metagene values calculated using datasets from Neve et al. (2006) Cancer Cell 10:515-527 (FIG. 4A), Garnett et al. (2012) Nature 483-570-575 (FIG. 4B); BRCA1 breast cancer signature as described in van't Veer et al. calculated using datasets from Neve et al. (2006) Cancer Cell 10:515-527 (FIG. 4C), and Garnett et al. (2012) Nature 483-570-575 (FIG. 4D).

### DETAILED DESCRIPTION

The methods and assays described herein are based, in part, on the discovery that a metagene value representing the expression of a combination of markers can be used to predict whether a subject will or will not respond to a cancer treatment comprising a PARP inhibitor and a DNA damage-inducing chemotherapeutic. Typically, the metagene value is based on normalized expression levels of the novel identified combination of the markers.

### DEFINITIONS

All scientific and technical terms used in this application have meanings commonly used in the art unless otherwise specified. As used in this application, the following words or phrases have the meanings specified.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable nucleic acid molecules are DNA, including genomic DNA or cDNA. Other suitable nucleic acid molecules are RNA, including mRNA.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g., the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, at least about a 20-fold increase, at least about a 50-fold increase, at least about a 100-fold increase, at least about a 1000-fold increase or more as compared to a reference level.

The term “statistically significant” or “significantly” refers to statistical significance and generally means two standard deviations (2SD) or more above or below normal or a reference. The term refers to statistical evidence that there is a difference. It is defined as the probability of making a decision to reject the null hypothesis when the null hypothesis is actually true. The decision is often made using the p-value.

As used herein, the term “transforming” or “transformation” refers to changing an object or a substance, e.g., biological sample, nucleic acid or protein, into another substance. The transformation can be physical, biological or chemical. Exemplary physical transformation includes, but not limited to, pre-treatment of a biological sample, e.g., from whole blood to blood serum by differential centrifugation. A biological/chemical transformation can involve at least one enzyme and/or a chemical reagent in a reaction. For example, a DNA sample can be digested into fragments by one or more restriction enzyme, or an exogenous molecule can be attached to a fragmented DNA sample with a ligase. In some embodiments, a DNA sample can undergo enzymatic replication, e.g., by polymerase chain reaction (PCR). In some embodiments an RNA transcript can be enzymatically reverse-transcribed to a cDNA sequence or a probe.

As used herein, a “probe” is defined as a molecule or moiety (e.g., a cDNA containing moiety) that specifically binds to a particular marker, e.g., in the present application, a probe is a molecule or moiety that specifically binds any one of the markers in Tables 1-4. Probes can include proteins, such as antigens derived from the biomarkers, antibodies, nucleic acid molecules, carbohydrates, lipids, ligands, drugs, ions and any other compound that can specifically bind to the markers contemplated for use with the methods and assays described herein. In one embodiment, a probe is a cDNA probe, such as probes useful in generating a gene expression array (e.g., a microarray).

As used herein, the phrase “a combination of at least the following markers,” at a minimum refers to assaying the expression levels for each of the following markers: EZR, SOX4, EMG1, NARS, SNRNP70, CLTA, EIF3B, EIF3E, UBAP2L, SF3A3, HK1, CKAP4, NOLC1, HCFC1, PPM1F, BMS1, AIMP2, POLG, MCM2, U2AF1, MOGS, SKIV2L, GCSH, CCND1, MLEC, RARS, SLC7A5, MANF, RAE1, CHD4, BYSL, GTF3C2, TRAP1, KIAA0114, BOP1, CDC25B, SF3B2, SMARCA4, DDX11, LMNB2, DHCR24, SAFB, CTPS, DUSP1, DDX10, CDC123, NDRG1, EIF2B2, FAM38A, SEC24C, RABEPK, FGFR3, SSRP1, and SLC1A3. Any number of additional markers such as those described in Tables 1, 2, and/or 3 or other markers (e.g., on a microarray) can be assayed with the combination of at least the markers noted above, however the expressional analysis or calculation of a metagene value is performed using the genes in the above-noted list or the combination of markers in any one of Tables 1-4. For example, a standard AFFYMETRIX™ array can be used to assay the expression levels of the above-noted markers, but the metagene value will be calculated for the combination of markers as outlined herein.

As used herein, the term “reference value” refers to a reference value, or range of values, obtained for one or more markers, a combination of markers, or a metagene value as described herein from e.g., at least one subject. In some embodiments, the reference value is obtained from the same subject prior to diagnosis of cancer or from a population of subjects that are substantially cancer free (e.g., evidence of cancerous cells or tumors are below detection levels for the cancer type). Alternatively, the reference value or range of values can be obtained from a subject or plurality of subjects determined to have a specific cancer. The reference sample can be stored as a value(s) on a computer or PDA device to permit comparison with a value obtained from a subject using the methods described herein. The reference sample can also be obtained from the same subject e.g., at an earlier time point prior to onset of a cancer or prior to initiation of treatment with a PARP inhibitor/DNA damage-inducing chemotherapeutic combination treatment using clinical tests known to those of skill in the art. One of skill in the art can determine an appropriate reference sample for use with the methods described herein.

As used herein, the term “reduced BRCA1 pathway function” refers to a reduction in the BRCA1 signaling pathway or BRCA1 DNA repair pathway in a biological sample or a cell by at least 20% compared to a biological sample or cell obtained from a subject substantially free of detectable cancer. In other embodiments, the phrase “reduced BRCA1 pathway function” refers to a reduction of the BRCA1 signaling or DNA repair pathway ina biological sample or cell by at at least 30%, at least 40%, at least 50%, at least 60%, at least 70%, at least 80%, at least 90%, at least 1-fold, at least 2-fold, at least 5-fold, at least 10-fold, at least 100-fold, at least 1000-fold or more as compared to a biological sample or cell obtained from a subject substantially free of detectable cancer. In one embodiment, the term “reduced BRCA1 pathway function” refers to the absence of detectable BRCA1 pathway function as assessed by e.g., assaying for BRCA-dependent DNA repair mechanisms. In another embodiment, the term “reduced BRCA1 pathway function” refers to an increase in the metagene value for the combination of markers in any one of Tables 1, 2, or 3, as that term is used herein.

As used herein, the term “metagene” refers to a pattern of gene expression of a combination of markers (i.e., not an actual gene). For example, a metagene refers to a pattern of gene expression of a combination of markers listed in any one of Tables 1-3.

As used herein, the term “metagene value” refers to a value or mathematical representation of expression of a combination of markers or metagene as that term is used herein. In one embodiment, the metagene value is an integer. In one embodiment, the metagene value is calculated using linear combination. In another embodiment, the metagene value is calculated using linear combination of normalized gene expression values, wherein the coefficient for each gene is 1.

As used herein, the terms “treat” “treatment” “treating,” or “amelioration” refer to therapeutic treatments, wherein the object is to reverse, alleviate, ameliorate, inhibit, slow down or stop the progression or severity of a condition associated with a disease or disorder, e.g., cancer, such as breast cancer or brain metastases. The term “treating” includes reducing or alleviating at least one adverse effect or symptom of a condition, disease or disorder associated with a cancer. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), reduced interventions, shortened hospital stays, and/or decreased mortality, whether detectable or undetectable. The term “treatment” of a disease also includes providing relief from the symptoms or side-effects of the disease (including palliative treatment). Treatment in this context does not include or encompass a complete “cure.”

As used herein, “pharmaceutically acceptable salt” refers to a salt that retains the desired biological activity of the parent compound and does not impart any undesired toxicological effects. Examples of such salts include, but are not limited to, (a) acid addition salts formed with inorganic acids, for example hydrochloric acid, hydrobromic acid, sulfuric acid, phosphoric acid, nitric acid and the like; and salts formed with organic acids such as, for example, acetic acid, oxalic acid, tartaric acid, succinic acid, maleic acid, furmaric acid, gluconic acid, citric acid, malic acid, ascorbic acid, benzoic acid, tannic acid, pamoic acid, alginic acid, polyglutamic acid, naphthalenesulfonic acids, naphthalenedisulfonic acids, polygalacturonic acid; (b) salts with polyvalent metal cations such as zinc, calcium, bismuth, barium, magnesium, aluminum, copper, cobalt, nickel, cadmium, and the like; or (c) salts formed with an organic cation formed from N,N′-dibenzylethylenediamine or ethylenediamine; or (d) combinations of (a) and (b) or (c), e.g., a zinc tannate salt; and the like. The preferred acid addition salts are the trifluoroacetate salt and the acetate salt.

The term “pharmaceutically acceptable” refers to compounds and compositions which can be administered to mammals without undue toxicity. The term “pharmaceutically acceptable carriers” excludes tissue culture medium.

As used herein, “pharmaceutically acceptable carrier” includes any material which, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. Preferred diluents for aerosol or parenteral administration are phosphate buffered saline or normal (0.9%) saline. Compositions comprising such carriers are formulated by well-known conventional methods (see, for example, Remington's Pharmaceutical Sciences, 18th edition, A. Gennaro, ed., Mack Publishing Co., Easton, Pa., 1990).

As used herein, the term “comprising” means that other elements can also be present in addition to the defined elements presented. The use of “comprising” indicates inclusion rather than limitation. For example, in the context of the present application, a metagene comprising a set of markers means a metagene wherein at least the set of indicated markers are included.

As used herein the term “consisting essentially of” refers to those elements required for a given embodiment. The term permits the presence of additional elements that do not materially affect the basic and novel or functional characteristic(s) of that embodiment of the invention. For example, in the context of the present application, a metagene consisting essentially of a set of markers means that the marker set includes the set of markers that have been determined to form the metagene but can include one or more markers that are not used in the calculations for the metagene value but are rather used as controls for background, or positive or negative expression or otherwise added to increase the accuracy of the assay but without including additional markers to the calculation of the metagene value.

The term “consisting of” refers to compositions, methods, and respective components thereof as described herein, which are exclusive of any element not recited in that description of the embodiment. For example, in the context of the present application, the metagene consisting of markers would mean that only the named markers are included in the metagene. The assay may still comprise other markers that may be needed for calculating the metagene value, such as markers for determining the background or markers that are used to control function of the assay or as positive or negative controls or to determine the normalizing expression background.

As used herein, “a” or “an” means at least one, unless clearly indicated otherwise. As used herein, to “prevent” or “protect against” a condition or disease means to hinder, reduce or delay the onset or progression of the condition or disease. Further, unless otherwise required by context, singular terms shall include pluralities and plural terms shall include the singular.

Other than in the operating examples, or where otherwise indicated, all numbers expressing quantities of ingredients or reaction conditions used herein should be understood as modified in all instances by the term “about.” The term “about” when used in connection with percentages can mean±1%.

It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims.

### Cancer

As described herein, we have identified a novel way of assaying a cell to determine whether BRCA1 pathway function in the cell had been reduced. Accordingly, the methods of selecting a treatment for cancer or methods of treating patients with reduced BRCA1 function are broadly applicable to essentially any cancer associated with reduced BRCA1 pathway function. Thus essentially any cancer or cancer cell population or cancer cell can be detected, or assayed, or diagnosed or treated according to the methods and assays described herein. Exemplary cancers, wherein the determination of or assaying for BRCA1 pathway function can be made to assist in treatment include, but are not limited to, bladder cancer; breast cancer; brain metastases; brain cancer including glioblastomas and medulloblastomas; cervical cancer; choriocarcinoma; colon cancer including colorectal carcinomas; endometrial cancer; esophageal cancer; gastric cancer; head and neck cancer; hematological neoplasms including acute lymphocytic and myelogenous leukemia, multiple myeloma, AIDS associated leukemias and adult T-cell leukemia lymphoma; intraepithelial neoplasms including Bowen's disease and Paget's disease, liver cancer; lung cancer including small cell lung cancer and non-small cell lung cancer; lymphomas including Hodgkin's disease and lymphocytic lymphomas; neuroblastomas; oral cancer including squamous cell carcinoma; osteosarcomas; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, synovial sarcoma and osteosarcoma; skin cancer including melanomas, Kaposi's sarcoma, basocellular cancer, and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullar carcinoma; transitional cancer and renal cancer including adenocarcinoma and Wilm's tumor.

### Biological Samples

As used herein, the term “biological sample” refers to a sample obtained from a subject that comprises a cell or population of cells or a quantity of tissue or fluid from a subject that comprises at least one cancer cell for which an analysis or assay can be performed to determine the function of the BRCA1 pathway. The sample typically has been removed from a subject, i.e., the biological sample is analyzed in vitro. In some aspects, the biological sample is assayed in vivo, i.e. without removal from the subject. A “biological sample” will contain cancer cells from a subject, but it can also comprise non-cellular biological material, such as non-cellular fractions of blood, saliva, or urine. As used herein, a “biological sample” or “tissue sample” refers to a sample of tissue or fluid isolated from an individual that comprises, consists essentially of or consists of cancer cells, including but not limited to, for example, blood, plasma, serum, tumor biopsy, circulating tumor cells, isolate dexosomes, urine, stool, sputum, spinal fluid, pleural fluid, nipple aspirates, lymph fluid, the external sections of the skin, respiratory, intestinal, and genitourinary tracts, tears, saliva, milk, cells (including but not limited to blood cells), tumors, organs, and also samples of in vitro cell culture constituent. In some embodiments, a biological sample is from a resection, bronchoscopic biopsy, or core needle biopsy of a primary, secondary or metastatic tumor, or a cellblock from pleural fluid. In addition, fine needle aspirate biological samples are also useful. In some embodiments, a biological sample is primary ascite cells. Samples can be fresh, frozen, fixed or optionally paraffin-embedded, frozen or subjected to other tissue preservation methods, including for example methods to preserve the phosphorylation status of polypeptides in the biological sample.

A biological sample can be provided by removing a sample of cells from subject, but can also be accomplished by using previously isolated cells (e.g., isolated by another person, at another time, and/or for another purpose), or by performing the methods of the invention in vivo. Archival tissues, such as those having treatment or outcome history can also be used. Biological samples also include tissue biopsies, cell culture. The biological sample can be pretreated as necessary for storage or preservation, by dilution in an appropriate buffer solution or concentrated, if desired. Any of a number of standard aqueous buffer solutions, employing one of a variety of buffers, such as phosphate, Tris, or the like, at physiological pH can be used. The biological sample can in certain circumstances be stored for use prior to use in the assay as disclosed herein. Such storage can be at +4° C. or frozen, for example at −20° C. or −80° C., provided suitable cryopreservation agents are used to maintain cell viability once the cells are thawed.

### Combinations of Markers for Expression Analysis

The methods, assays and compositions contemplated herein rely, in part, on assaying the expression level of a combination of genes e.g., that indicate reduced function of the BRCA1 pathway in a cancer cell. Tables 1, 2, and 3 show exemplary marker combinations that can be used to permit the selection of a treatment for a cancer, to classify a cancer subject into a subgroup, and to determine reduced function of the BRCA1 pathway in a cancer cell that can provide guidance in selection of the appropriate treatment for the patient. Table 3 shows an exemplary combination of control markers that can be used to normalize the expression measurements for the combinations of markers in Tables 1, 2, or 3.

TABLE 1
Genes with reduced expression in cancers having reduced function
of the BRCA1 pathway.

| MSigDB | Entrez | Official | | GenBank |
|---|---|---|---|---|
| Symbol | GeneID | Symbol | ID_REF | Accession No. |
| FGFR3 | 2261 | FGFR3 | 204379_s_at | NM_000142 |
| | | | 204380_s_at | |
| SFRS11 | 9295 | SRSF11 | 200685_at | NM_004768 |
| | | | 200686_s_at | Contig33298_RC |
| | | | 213742_at | Contig52308_RC |
| | | | | Contig42005_RC |
| VIL2 | 7430 | EZR | 208621_s_at | NM_003379 |
| | | | 208622_s_at | |
| | | | 208623_s_at | |
| | | | 217230_at | |
| | | | 217234_s_at | |
| PKN1 | 5585 | PKN1 | 202161_at | NM_002741 |
| SOX4 | 6659 | SOX4 | 201416_at | NM_003107 |
| | | | 201417_at | |
| | | | 201418_s_at | |
| | | | 213665_at | |
| | | | 213668_s_at | |
| EMG1 | 10436 | EMG1 | 209233_at | U72514 |
| RELA | 5970 | RELA | 201783_s_at | L19067 |
| | | | 209878_s_at | |
| JTV1 | 7965 | AIMP2 | 202138_x_at | NM_006303 |
| | | | 209971_x_at | |
| | | | 209972_s_at | |
| NARS | 4677 | NARS | 200027_at | NM_004539 |
| HSP90B1 | 7184 | HSP90B1 | 200598_s_at | NM_003299 |
| | | | 200599_s_at | |
| | | | 216449_x_at | |
| | | | 216450_x_at | |
| SNRP70 | 6625 | SNRNP70 | 201221_s_at | NM_003089 |
| | | | 213121_at | |
| NME3 | 4832 | NME3 | 204862_s_at | |
| CLTA | 1211 | CLTA | 200960_x_at | NM_001833 |
| | | | 204050_s_at | NM_007096 |
| | | | 216293_at | |
| | | | 216295_s_at | |
| | | | 216296_at | |
| CAD | 790 | CAD | 202715_at | NM_004341 |
| EIF3S9 | 8662 | EIF3B | 203462_x_at | NM_003751 |
| | | | 208688_x_at | Contig62628_RC |
| | | | 211501_s_at | |
| EIF3S6 | 3646 | EIF3E | 208697_s_at | NM_001568 |
| UBAP2L | 9898 | UBAP2L | 201377_at | NM_014847 |
| | | | 201378_s_at | Contig112_RC |
| | | | 209947_at | |
| | | | 214695_at | |
| SF3A3 | 10946 | SF3A3 | 203818_s_at | NM_006802 |
| HK1 | 3098 | HK1 | 200697_at | NM_000188 |
| TCOF1 | 6949 | TCOF1 | 202384_s_at | NM_000356 |
| | | | 202385_s_at | |
| CKAP4 | 10970 | CKAP4 | 200998_s_at | NM_006825 |
| | | | 200999_s_at | |
| CCNE1 | 898 | CCNE1 | 213523_at | M74093 |
| NOLC1 | 9221 | NOLC1 | 205895_s_at | NM_004741 |
| | | | 211949_s_at | |
| | | | 211951_at | |
| HCFC1 | 3054 | HCFC1 | 202473_x_at | NM_005334 |
| | | | 202474_s_at | |
| PPM1F | 9647 | PPM1F | 203063_at | NM_014634 |
| | | | 37384_at | |
| BMS1L | 9790 | BMS1 | 203082_at | NM_014753 |
| RASSF7 | 8045 | RASSF7 | 204927_at | NM_003475 |
| | | | 40359_at | |
| FDFT1 | 2222 | FDFT1 | 208647_at | NM_004462 |
| | | | 210950_s_at | |
| FARSLA | 2193 | FARSA | 202159_at | NM_004461 |
| | | | 216602_s_at | |
| POLG | 5428 | POLG | 203366_at | NM_002693 |
| | | | 217635_s_at | |
| | | | 217636_at | |
| MCM2 | 4171 | MCM2 | 202107_s_at | NM_004526 |
| VDAC1 | 7416 | VDAC1 | 212038_s_at | NM_003374 |
| | | | 217139_at | Contig28450_RC |
| | | | 217140_s_at | |
| U2AF1 | 7307 | U2AF1 | 202858_at | NM_006758 |
| GCS1 | 7841 | MOGS | 210627_s_at | NM_006302 |
| ATIC | 471 | ATIC | 208758_at | NM_004044 |
| CHAF1A | 10036 | CHAF1A | 203975_s_at | NM_005483 |
| | | | 203976_s_at | |
| | | | 214426_x_at | |
| SKIV2L | 6499 | SKIV2L | 203727_at | NM_006929 |
| GCSH | 2653 | GCSH | 213129_s_at | NM_004483 |
| | | | 213133_s_at | |
| GAL | 51083 | GAL | 207466_at | NM_015973 |
| | | | 214240_at | |
| CCDC85B | 11007 | CCDC85B | 204609_at | |
| | | | 204610_s_at | |
| | | | 213250_at | |
| CCND1 | 595 | CCND1 | 208711_s_at | |
| | | | 208712_at | |
| KIAA0152 | 9761 | MLEC | 200616_s_at | NM_014730 |
| | | | 200617_at | |
| RARS | 5917 | RARS | 201330_at | NM_002887 |
| WBSCR1 | 7458 | EIF4H | 206621_s_at | D26068 |
| FKBP4 | 2288 | FKBP4 | 200894_s_at | NM_002014 |
| | | | 200895_s_at | |
| SLC7A5 | 8140 | SLC7A5 | 201195_s_at | NM_003486 |
| MTHFD1 | 4522 | MTHFD1 | 202309_at | NM_005956 |
| NSDHL | 50814 | NSDHL | 209279_s_at | NM_015922 |
| | | | 215093_at | |
| PWP2H | 5822 | PWP2 | 209336_at | NM_005049 |
| FASN | 2194 | FASN | 212218_s_at | NM_004104 |
| | | | 217006_x_at | |
| ARMET | 7873 | MANF | 202655_at | NM_006010 |
| EIF4G1 | 1981 | EIF4G1 | 208624_s_at | NM_004953 |
| | | | 208625_s_at | |
| JARID1C | 8242 | KDM5C | 202383_at | L25270 |
| SCRIB | 23513 | SCRIB | 212556_at | D63481 |
| SCAP | 22937 | SCAP | 212329_at | D83782 |
| PSMD1 | 5707 | PSMD1 | 201198_s_at | NM_002807 |
| | | | 201199_s_at | |
| RAE1 | 8480 | RAE1 | 201558_at | NM_003610 |
| | | | 211318_s_at | |
| | | | 211319_at | |
| CHD4 | 1108 | CHD4 | 201182_s_at | NM_001273 |
| | | | 201183_s_at | |
| | | | 201184_s_at | |
| SLC29A1 | 2030 | SLC29A1 | 201801_s_at | NM_004955 |
| | | | 201802_at | |
| BYSL | 705 | BYSL | 203612_at | NM_004053 |
| GTF3C2 | 2976 | GTF3C2 | 204366_s_at | NM_001521 |
| | | | 210620_s_at | |
| | | | 212429_s_at | |
| TRAP1 | 10131 | TRAP1 | 201391_at | NM_016292 |
| PRKACA | 5566 | PRKACA | 202801_at | NM_002730 |
| | | | 216234_s_at | |
| KIAA0179 | 23076 | RRP1B | 212844_at | D80001 |
| | | | 212846_at | |
| KIAA0114 | 57291 | DANCR | | Contig57239_RC |
| POLD1 | 5424 | POLD1 | 203422_at | NM_002691 |
| DNTTIP2 | 30836 | DNTTIP2 | 202776_at | NM_014597 |
| BOP1 | 23246 | BOP1 | 212563_at | D50914 |
| | | | 216397_s_at | |
| CDC25B | 994 | CDC25B | 201853_s_at | NM_004358 |
| MRPS12 | 6183 | MRPS12 | 204330_s_at | Y11681 |
| | | | 204331_s_at | |
| | | | 210008_s_at | |
| | | | 213840_s_at | |
| SF3B2 | 10992 | SF3B2 | 200619_at | NM_006842 |
| NUP214 | 8021 | NUP214 | 202155_s_at | NM_005085 |
| | | | 211261_at | Contig43761_RC |
| PHKG2 | 5261 | PHKG2 | 203709_at | Contig15439_RC |
| | | | | NM_000294 |
| AGPAT2 | 10555 | AGPAT2 | 210678_s_at | NM_006412 |
| | | | 32837_at | |
| SMARCA4 | 6597 | SMARCA4 | 208793_x_at | NM_003072 |
| | | | 208794_s_at | |
| | | | 212520_s_at | |
| | | | 213719_s_at | |
| | | | 213720_s_at | |
| | | | 214360_at | |
| | | | 214728_x_at | |
| | | | 215714_s_at | |
| | | | 217656_at | |
| PPAT | 5471 | PPAT | 209433_s_at | NM_002703 |
| | | | 209434_s_at | |
| SNRPA1 | 6627 | SNRPA1 | 206055_s_at | NM_003090 |
| | | | 215722_s_at | |
| | | | 216977_x_at | |
| DDX11 | 1663 | DDX11 | 208149_x_at | NM_004399 |
| | | | 208159_x_at | U75968 |
| | | | 210206_s_at | |
| | | | 213378_s_at | |
| RPIA | 22934 | RPIA | 212973_at | L35035 |
| ALG3 | 10195 | ALG3 | 207396_s_at | NM_005787 |
| LMNB2 | 84823 | LMNB2 | 216952_s_at | M94362 |
| TAF1C | 9013 | TAF1C | 203937_s_at | NM_005679 |
| | | | 203938_s_at | |
| DDT | 1652 | DDT | 202929_s_at | NM_001355 |
| DHCR24 | 1718 | DHCR24 | 200862_at | D13643 |
| SAFB | 6294 | SAFB | 201747_s_at | NM_002967 |
| | | | 201748_s_at | |
| | | | 213635_s_at | |
| CDC37 | 11140 | CDC37 | 209953_s_at | NM_007065 |
| H1FX | 8971 | H1FX | 204805_s_at | NM_006026 |
| AP3D1 | 8943 | AP3D1 | 206592_s_at | NM_003938 |
| | | | 208710_s_at | |
| | | | 210974_s_at | |
| DNAJC7 | 7266 | DNAJC7 | 202416_at | NM_003315 |
| HSF1 | 3297 | HSF1 | 202344_at | NM_005526 |
| | | | 213756_s_at | |
| CTPS | 1503 | CTPS1 | 202613_at | NM_001905 |
| SSRP1 | 6749 | SSRP1 | 200956_s_at | NM_003146 |
| | | | 200957_s_at | |
| DUSP1 | 1843 | DUSP1 | 201041_s_at | NM_004417 |
| | | | 201044_x_at | |
| DDX10 | 1662 | DDX10 | 204977_at | NM_004398 |
| PDCD11 | 22984 | PDCD11 | 212422_at | D80007 |
| | | | 212424_at | |
| C10ORF7 | 8872 | CDC123 | 201725_at | NM_006023 |
| RXRA | 6256 | RXRA | 202426_s_at | NM_002957 |
| | | | 202449_s_at | |
| NDRG1 | 10397 | NDRG1 | 200632_s_at | NM_006096 |
| | | | | Contig27182_RC |
| EIF2B2 | 8892 | EIF2B2 | 202461_at | NM_014239 |
| ANM11 | 311 | ANXA11 | 206200_s_at | NM_001157 |
| | | | 214783_s_at | |
| FAM38A | 9780 | PIEZO1 | 202771_at | |
| XRCC5 | 7520 | XRCC5 | 208642_s_at | NM_021141 |
| | | | 208643_s_at | M30938 |
| SEC24C | 9632 | SEC24C | 202361_at | NM_004922 |
| PMPCA | 23203 | PMPCA | 212088_at | D21064 |
| POLRMT | 5442 | POLRMT | 203782_s_at | NM_005035 |
| | | | 203783_x_at | |
| RABEPK | 10244 | RABEPK | 203150_at | NM_005833 |
| SMAD6 | 4091 | SMAD6 | 207069_s_at | NM_005585 |
| | | | 209886_s_at | |
| | | | 209887_at | |
| | | | 213565_s_at | |
| NMT1 | 4836 | NMT1 | 201157_s_at | AF043324 |
| | | | 201158_at | NM_021079 |
| | | | 201159_s_at | |
| SON | 6651 | SON | 201085_s_at | NM_003103 |
| | | | 201086_x_at | |
| | | | 213538_at | |
| | | | 214988_s_at | |
| MAP2K5 | 5607 | MAP2K5 | 204756_at | NM_002757 |
| | | | 210482_x_at | |
| | | | 211370_s_at | |
| | | | 211371_at | |
| | | | 216435_at | |
| | | | 216765_at | |
| SLC1A3 | 6507 | SLC1A3 | 202800_at | NM_004172 |
| BRD2 | 6046 | BRD2 | 208685_x_at | NM_005104 |
| | | | 208686_s_at | |
| | | | 214911_s_at | |

TABLE 2
An alternative list of genes with reduced expression in cancers having
reduced function of the BRCA1 pathway.

| | Entrez | | Entrez | | Entrez |
|---|---|---|---|---|---|
| Official | Gene | Official | Gene | Official | Gene |
| symbol | ID | symbol | ID | symbol | ID |
| XRCC5 | 7520 | SLC29A1 | 2030 | SKIV2L | 6499 |
| CCDC85B | 11007 | GCSH | 2653 | HK1 | 3098 |
| CTPS | 1503 | NDRG1 | 10397 | HCFC1 | 3054 |
| BMS1 | 9790 | UBAP2L | 9898 | SF3B2 | 10992 |
| MANF | 7873 | SFRS11 | 9295 | NUP214 | 8021 |
| H1FX | 8971 | SMAD6 | 4091 | ATIC | 471 |
| SLC7A5 | 8140 | CKAP4 | 10970 | SAFB | 6294 |
| FDFT1 | 2222 | EIF4H | 7458 | FASN | 2194 |
| DDX11 | 1663 | VDAC1 | 7416 | AP3D1 | 8943 |
| RAE1 | 8480 | MOGS | 7841 | RPIA | 22934 |
| U2AF1 | 7307 | NMT1 | 4836 | GTF3C2 | 2976 |
| DNAJC7 | 7266 | POLRMT | 5442 | CHD4 | 1108 |
| EIF3E | 3646 | NOLC1 | 9221 | NSDHL | 50814 |
| EIF3B | 8662 | POLD1 | 5424 | SSRP1 | 6749 |
| PRKACA | 5566 | CHAF1A | 10036 | BRD2 | 6046 |
| DDX10 | 1662 | TAF1C | 9013 | PDCD11 | 22984 |
| EIF2B2 | 8892 | CLTA | 1211 | SNRNP70 | 6625 |
| MAP2K5 | 5607 | NARS | 4677 | KIAA0114 | 57291 |
| SNRPA1 | 6627 | TCOF1 | 6949 | SF3A3 | 10946 |
| POLG | 5428 | ALG3 | 10195 | CDC25B | 994 |
| EMG1 | 10436 | CAD | 790 | SON | 6651 |
| RELA | 5970 | RASSF7 | 8045 | HSP90B1 | 7184 |
| BYSL | 705 | MTHFD1 | 4522 | DUSP1 | 1843 |
| RXRA | 6256 | CCNE1 | 898 | ANXA11 | 311 |
| KDM5C | 8242 | SLC1A3 | 6507 | | |
| CDC123 | 8872 | 1-15F1 | 3297 | | |
| AIMP2 | 7965 | PSMD1 | 5707 | | |
| PWP2 | 5822 | SEC24C | 9632 | | |
| NME3 | 4832 | AGPAT2 | 10555 | | |
| RARS | 5917 | DHCR24 | 1718 | | |
| FARSA | 2193 | EZR | 7430 | | |
| DNTTIP2 | 30836 | PHKG2 | 5261 | | |
| FAM38A | 9780 | PKN1 | 5585 | | |
| SMARCA4 | 6597 | MCM2 | 4171 | | |
| FGFR3 | 2261 | GAL | 51083 | | |
| LMNB2 | 84823 | SCAP | 22937 | | |
| MRPS12 | 6183 | PPM1F | 9647 | | |
| RABEPK | 10244 | EIF4G1 | 1981 | | |
| SOX4 | 6659 | TRAP1 | 10131 | | |
| BOP1 | 23246 | CCND1 | 595 | | |
| MLEC | 9761 | DDT | 1652 | | |
| SCRIB | 23513 | PMPCA | 23203 | | |
| PPAT | 5471 | RRP1B | 23076 | | |
| CDC37 | 11140 | FKBP4 | 2288 | | |

TABLE 3
BRCA1_OVEREXP_CE gene set

| Official | Entrez | Official | Entrez |
|---|---|---|---|
| symbol | Gene ID | symbol | Gene ID |
| CTPS | 1503 | UBAP2L | 9898 |
| BM S1 | 9790 | CKAP4 | 10970 |
| MANF | 7873 | MOGS | 7841 |
| SLC7A5 | 8140 | NOLC1 | 9221 |
| DDX11 | 1663 | CLTA | 1211 |
| RAE1 | 8480 | NARS | 4677 |
| U2AF1 | 7307 | SLC1A3 | 6507 |
| EIF3E | 3646 | SEC24C | 9632 |
| EIF3B | 8662 | DHCR24 | 1718 |
| DDX10 | 1662 | EZR | 7430 |
| EIF2B2 | 8892 | MCM2 | 4171 |
| POLG | 5428 | PPM1F | 9647 |
| EMG1 | 10436 | TRAP1 | 10131 |
| BYSL | 705 | CCND1 | 595 |
| CDC123 | 8872 | SKIV2L | 6499 |
| AIMP2 | 7965 | HK1 | 3098 |
| RARS | 5917 | HCFC1 | 3054 |
| FAM38A | 9780 | SF3B2 | 10992 |
| SMARCA4 | 6597 | SAFB | 6294 |
| FGFR3 | 2261 | GTF3C2 | 2976 |
| LMNB2 | 84823 | CHD4 | 1108 |
| RABEPK | 10244 | SSRP1 | 6749 |
| SOX4 | 6659 | SNRNP70 | 6625 |
| BOP1 | 23246 | KIAA0114 | 57291 |
| MLEC | 9761 | SF3A3 | 10946 |
| GCSH | 2653 | CDC25B | 994 |
| NDRG1 | 10397 | DUSP1 | 1843 |

One of skill in the art will appreciate that one need not necessarily use all of the markers listed in each of Tables 1, 2, and 3 with the methods and assays described herein. For example, in some aspects, the methods and assays can be performed herein using at least five markers from Tables 1, 2, or 3; in other embodiments, the methods and assays described herein are performed using expression levels for at least 6, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 110 markers or more of Tables 1, 2, or 3. In one embodiment, the methods and assays described herein are performed by measuring expression of all of the markers listed in each of Tables, 1, 2 or 3.

In some embodiments, the methods and assays are performed herein by assaying expression levels for all of the markers in Table 3 and at least one additional non-duplicative marker from Table 1, or Table 2. In other embodiments, the methods and assays are performed herein by assaying expression levels for all of the markers in Table 3 and at least 2, at least 3, at least 4, at least 5, at least 6, at least 7, at least 8, at least 9, at least 10, at least 15, at least 20, at least 25, at least 30, at least 35, at least 40, or at least 45 (e.g., 46, 47, or 48) additional, non-duplicative markers from Table 1 or Table 2.

The level of expression of a combination of markers in Tables 1, 2 or 3, which is higher than a reference level by at least about 10%, at least about 20%, at least about 30%, at least about 40%, at least about 50%, at least about 80%, at least about 100%, at least about 200%, at least about 300%, at least about 500% or at least about 1000% or more, is indicative that the subject is likely to benefit from combined treatment with a PARP inhibitor and a DNA damage-inducing chemotherapeutic and/or that the subject should be administered a PARP inhibitor and a DNA damage-inducing chemotherapeutic in accordance with the methods described herein. Therefore, when an increased metagene value is detected or indicated in the cancer cell from the patient, the patient can be administered a combined cancer treatment with a PARP inhibitor and a DNA damage-inducing chemotherapeutic and more likely than not expected to benefit from the treatment. If no increase in the metagene value is seen in a cancer cell sample from the subject, a combined treatment with a PARP inhibitor and a DNA damage-inducing chemotherapeutic is contraindicated, and thus should not be administered as it would more likely than not be expected to result in side effects without benefit to the subject.

Measuring Protein and mRNA Expression of a Combination of Markers

Methods to measure gene expression products associated with the marker genes described herein are well known to a skilled artisan. Such methods to measure gene expression products, e.g., protein level, include ELISA (enzyme linked immunosorbent assay), Western blot, and immunoprecipitation, immunofluorescence using detection reagents such as an antibody or protein binding agents. Alternatively, a peptide can be detected in a subject by introducing into a subject a labeled anti-peptide antibody and other types of detection agent. For example, the antibody can be labeled with a radioactive marker whose presence and location in the subject is detected by standard imaging techniques.

For example, antibodies for the polypeptide expression products of the marker genes described herein are commercially available and can be used for the purposes of the invention to measure protein expression levels. Alternatively, since the amino acid sequences for the marker genes described herein are known and publically available at NCBI website, one of skill in the art can raise their own antibodies against these proteins of interest for the purpose of the invention. The amino acid sequences of the marker genes described herein have been assigned NCBI accession numbers for different species such as human, mouse and rat.

In some embodiments, immunohistochemistry (“IHC”) and immunocytochemistry (“ICC”) techniques can be used to assay expression levels of a combination of markers as described herein. IHC is the application of immunochemistry to tissue sections, whereas ICC is the application of immunochemistry to cells or tissue imprints after they have undergone specific cytological preparations such as, for example, liquid-based preparations Immunochemistry is a family of techniques based on the use of an antibody, wherein the antibodies are used to specifically target molecules inside or on the surface of cells. The antibody typically contains a marker that undergoes a biochemical reaction, and thereby experiences a change in color upon encountering the targeted molecules. In some instances, signal amplification can be integrated into the particular protocol, wherein a secondary antibody, that includes the marker stain or marker signal, follows the application of a primary specific antibody.

In some embodiments, the assay can be a Western blot analysis. Alternatively, proteins can be separated by two-dimensional gel electrophoresis systems. Two-dimensional gel electrophoresis is well known in the art and typically involves iso-electric focusing along a first dimension followed by SDS-PAGE electrophoresis along a second dimension. These methods can also require a considerable amount of cellular material. The analysis of 2D SDS-PAGE gels can be performed by determining the intensity of protein spots on the gel, or can be performed using immune detection. In other embodiments, protein samples are analyzed by mass spectroscopy.

Immunological tests can be used with the methods and assays described herein and include, for example, competitive and non-competitive assay systems using techniques such as Western blots, radioimmunoassay (RIA), ELISA (enzyme linked immunosorbent assay), “sandwich” immunoassays, immunoprecipitation assays, immunodiffusion assays, agglutination assays, e.g. latex agglutination, complement-fixation assays, immunoradiometric assays, fluorescent immunoassays, e.g. FIA (fluorescence-linked immunoassay), chemiluminescence immunoassays (CLIA), electrochemiluminescence immunoassay (ECLIA, counting immunoassay (CIA), lateral flow tests or immunoassay (LFIA), magnetic immunoassay (MIA), and protein A immunoassays. Methods for performing such assays are known in the art, provided an appropriate antibody reagent is available. In some embodiment, the immunoassay can be a quantitative or a semi-quantitative immunoassay.

An immunoassay is a biochemical test that measures the concentration of a substance in a biological sample, typically a fluid sample such as serum, using the interaction of an antibody or antibodies to its antigen. The assay takes advantage of the highly specific binding of an antibody with its antigen. For the methods and assays described herein, specific binding of the target polypeptides with respective proteins or protein fragments, or an isolated peptide, or a fusion protein described herein occurs in the immunoassay to form a target protein/peptide complex. The complex is then detected by a variety of methods known in the art. An immunoassay also often involves the use of a detection antibody.

Enzyme-linked immunosorbent assay, also called ELISA, enzyme immunoassay or EIA, is a biochemical technique used mainly in immunology to detect the presence of an antibody or an antigen in a sample. The ELISA has been used as a diagnostic tool in medicine and plant pathology, as well as a quality control check in various industries.

In one embodiment, an ELISA involving at least one antibody with specificity for the particular desired antigen (e.g., a marker gene polypeptide as described herein) can also be performed. A known amount of sample and/or antigen is immobilized on a solid support (e.g., a polystyrene micro titer plate). Immobilization can be either non-specific (e.g., by adsorption to the surface) or specific (e.g., where another antibody immobilized on the surface is used to capture antigen or a primary antibody). After the antigen is immobilized, the detection antibody is added, forming a complex with the antigen. The detection antibody can be covalently linked to an enzyme, or can itself be detected by a secondary antibody which is linked to an enzyme through bio-conjugation. Between each step the plate is typically washed with a mild detergent solution to remove any proteins or antibodies that are not specifically bound. After the final wash step the plate is developed by adding an enzymatic substrate to produce a visible signal, which indicates the quantity of antigen in the sample. Older ELISAs utilize chromogenic substrates, though newer assays employ fluorogenic substrates with much higher sensitivity.

In another embodiment, a competitive ELISA is used. Purified antibodies that are directed against a target polypeptide or fragment thereof are coated on the solid phase of multi-well plate, e.g., conjugated to a solid surface. A second batch of purified antibodies that are not conjugated on any solid support is also needed. These non-conjugated purified antibodies are labeled for detection purposes, for example, labeled with horseradish peroxidase to produce a detectable signal. A sample (e.g., tumor, blood, serum or urine) from a subject is mixed with a known amount of desired antigen (e.g., a known volume or concentration of a sample comprising a target polypeptide) together with the horseradish peroxidase labeled antibodies and the mixture is then are added to coated wells to form competitive combination. After incubation, if the polypeptide level is high in the sample, a complex of labeled antibody reagent-antigen will form. This complex is free in solution and can be washed away. Washing the wells will remove the complex. Then the wells are incubated with TMB (3, 3′,5, 5′-tetramethylbenzidene) color development substrate for localization of horseradish peroxidase-conjugated antibodies in the wells. There will be no color change or little color change if the target polypeptide level is high in the sample. If there is little or no target polypeptide present in the sample, a different complex in formed, the complex of solid support bound antibody reagents-target polypeptide. This complex is immobilized on the plate and is not washed away in the wash step. Subsequent incubation with TMB will produce much color change. Such a competitive ELISA test is specific, sensitive, reproducible and easy to operate.

There are other different forms of ELISA, which are well known to those skilled in the art. The standard techniques known in the art for ELISA are described in “Methods in Immunodiagnosis”, 2nd Edition, Rose and Bigazzi, eds. John Wiley & Sons, 1980; and Oellerich, M. 1984, J. Clin. Chem. Clin. Biochem. 22:895-904, which are incorporated herein by reference in their entirety.

In one embodiment, the levels of a polypeptide in a sample can be detected by a lateral flow immunoassay test (LFIA), also known as the immunochromatographic assay, or strip test. LFIAs are a simple device intended to detect the presence (or absence) of antigen, e.g. a polypeptide, in a fluid sample. There are currently many LFIA tests are used for medical diagnostics either for home testing, point of care testing, or laboratory use. LFIA tests are a form of immunoassay in which the test sample flows along a solid substrate via capillary action. After the sample is applied to the test strip it encounters a colored reagent (generally comprising antibody specific for the test target antigen) bound to microparticles which mixes with the sample and transits the substrate encountering lines or zones, which have been pretreated with another antibody or antigen. Depending upon the level of target polypeptides present in the sample the colored reagent can be captured and become bound at the test line or zone. LFIAs are essentially immunoassays adapted to operate along a single axis to suit the test strip format or a dipstick format. Strip tests are extremely versatile and can be easily modified by one skilled in the art for detecting an enormous range of antigens from fluid samples such as urine, blood, water, and/or homogenized tumor samples etc. Strip tests are also known as dip stick tests, the name bearing from the literal action of “dipping” the test strip into a fluid sample to be tested. LFIA strip tests are easy to use, require minimal training and can easily be included as components of point-of-care test (POCT) diagnostics to be used on site in the field. LFIA tests can be operated as either competitive or sandwich assays. Sandwich LFIAs are similar to sandwich ELISA. The sample first encounters colored particles which are labeled with antibodies raised to the target antigen. The test line will also contain antibodies to the same target, although it may bind to a different epitope on the antigen. The test line will show as a colored band in positive samples. In some embodiments, the lateral flow immunoassay can be a double antibody sandwich assay, a competitive assay, a quantitative assay or variations thereof. Competitive LFIAs are similar to competitive ELISA. The sample first encounters colored particles which are labeled with the target antigen or an analogue. The test line contains antibodies to the target/its analogue. Unlabelled antigen in the sample will block the binding sites on the antibodies preventing uptake of the colored particles. The test line will show as a colored band in negative samples. There are a number of variations on lateral flow technology. It is also possible to apply multiple capture zones to create a multiplex test.

The use of “dip sticks” or LFIA test strips and other solid supports have been described in the art in the context of an immunoassay for a number of antigen biomarkers. U.S. Pat. Nos. 4,943,522; 6,485,982; 6,187,598; 5,770,460; 5,622,871; 6,565,808, U.S. patent application Ser. No. 10/278,676; U.S. Ser. No. 09/579,673 and U.S. Ser. No. 10/717,082, which are incorporated herein by reference in their entirety, are non-limiting examples of such lateral flow test devices. Examples of patents that describe the use of “dip stick” technology to detect soluble antigens via immunochemical assays include, but are not limited to U.S. Pat. Nos. 4,444,880; 4,305,924; and 4,135,884; which are incorporated by reference herein in their entireties. The apparatuses and methods of these three patents broadly describe a first component fixed to a solid surface on a “dip stick” which is exposed to a solution containing a soluble antigen that binds to the component fixed upon the “dip stick,” prior to detection of the component-antigen complex upon the stick. It is within the skill of one in the art to modify the teachings of this “dip stick” technology for the detection of polypeptides using antibody reagents as described herein.

Other techniques can be used to detect the level of a polypeptide in a sample. One such technique is the dot blot, and adaptation of Western blotting (Towbin et at., Proc. Nat. Acad. Sci. 76:4350 (1979)). In a Western blot, the polypeptide or fragment thereof can be dissociated with detergents and heat, and separated on an SDS-PAGE gel before being transferred to a solid support, such as a nitrocellulose or PVDF membrane. The membrane is incubated with an antibody reagent specific for the target polypeptide or a fragment thereof. The membrane is then washed to remove unbound proteins and proteins with non-specific binding. Detectably labeled enzyme-linked secondary or detection antibodies can then be used to detect and assess the amount of polypeptide in the sample tested. The intensity of the signal from the detectable label corresponds to the amount of enzyme present, and therefore the amount of polypeptide. Levels can be quantified, for example by densitometry.

In certain embodiments, the gene expression products as described herein can be instead determined by determining the level of messenger RNA (mRNA) expression of genes associated with the marker genes described herein. Such molecules can be isolated, derived, or amplified from a biological sample, such as a tumor biopsy. Detection of mRNA expression is known by persons skilled in the art, and comprise, for example but not limited to, PCR procedures, RT-PCR, Northern blot analysis, differential gene expression, RNA protection assay, microarray analysis, hybridization methods, next-generation sequencing etc. Non-limiting examples of next-generation sequencing technologies can include Ion Torrent, Illumina, SOLiD, 454; Massively Parallel Signature Sequencing solid-phase, reversible dye-terminator sequencing; and DNA nanoball sequencing. In one embodiment, the gene expression products are measured using a targeted multiplex platform and/or a barcoding technology such as e.g., NANOSTRING NCOUNTER™.

In general, the PCR procedure describes a method of gene amplification which is comprised of (i) sequence-specific hybridization of primers to specific genes or sequences within a nucleic acid sample or library, (ii) subsequent amplification involving multiple rounds of annealing, elongation, and denaturation using a thermostable DNA polymerase, and (iii) screening the PCR products for a band of the correct size. The primers used are oligonucleotides of sufficient length and appropriate sequence to provide initiation of polymerization, e.g., each primer is specifically designed to be complementary to a strand of the genomic locus to be amplified. In an alternative embodiment, mRNA level of gene expression products described herein can be determined by reverse-transcription (RT) PCR and by quantitative RT-PCR (QRT-PCR) or real-time PCR methods. Methods of RT-PCR and QRT-PCR are well known in the art. The nucleic acid sequences of the marker genes described herein have been assigned NCBI accession numbers for different species such as human, mouse and rat. Accordingly, a skilled artisan can design an appropriate primer based on the known sequence for determining the mRNA level of the respective gene.

Nucleic acid and ribonucleic acid (RNA) molecules can be isolated from a particular biological sample using any of a number of procedures, which are well-known in the art, the particular isolation procedure chosen being appropriate for the particular biological sample. For example, freeze-thaw and alkaline lysis procedures can be useful for obtaining nucleic acid molecules from solid materials; heat and alkaline lysis procedures can be useful for obtaining nucleic acid molecules from urine; and proteinase K extraction can be used to obtain nucleic acid from blood (Roiff, A et al. PCR: Clinical Diagnostics and Research, Springer (1994)).

In general, the PCR procedure describes a method of gene amplification which is comprised of (i) sequence-specific hybridization of primers to specific genes within a nucleic acid sample or library, (ii) subsequent amplification involving multiple rounds of annealing, elongation, and denaturation using a DNA polymerase, and (iii) screening the PCR products for a band of the correct size. The primers used are oligonucleotides of sufficient length and appropriate sequence to provide initiation of polymerization, e.g., each primer is specifically designed to be complementary to each strand of the nucleic acid molecule to be amplified.

In an alternative embodiment, mRNA level of gene expression products described herein can be determined by reverse-transcription (RT) PCR and by quantitative RT-PCR (QRT-PCR) or real-time PCR methods. Methods of RT-PCR and QRT-PCR are well known in the art.

In one embodiment, the NanoString nCounter® Analysis System can be used to determine the expression levels of any or all of the genes described above. The NanoString nCounter® Analysis System permits direct, multiplexed measurements of gene expression through digital readouts of the relative abundance of hundreds of mRNA transcripts. The nCounter® Analysis System uses gene-specific probe pairs that hybridize directly to the mRNA sample in solution, eliminating any enzymatic reactions that might introduce bias in the results. The NanoString nCounter® Analysis System is available commercially from e.g., NANOSTRING TECHNOLOGIES™ (Seattle, Wash.).

In some embodiments, one or more of the reagents (e.g. an antibody reagent and/or nucleic acid probe) described herein can comprise a detectable label and/or comprise the ability to generate a detectable signal (e.g. by catalyzing reaction converting a compound to a detectable product). Detectable labels can comprise, for example, a light-absorbing dye, a fluorescent dye, or a radioactive label. Detectable labels, methods of detecting them, and methods of incorporating them into reagents (e.g. antibodies and nucleic acid probes) are well known in the art.

In some embodiments, detectable labels can include labels that can be detected by spectroscopic, photochemical, biochemical, immunochemical, electromagnetic, radiochemical, or chemical means, such as fluorescence, chemifluoresence, or chemiluminescence, or any other appropriate means. The detectable labels used in the methods described herein can be primary labels (where the label comprises a moiety that is directly detectable or that produces a directly detectable moiety) or secondary labels (where the detectable label binds to another moiety to produce a detectable signal, e.g., as is common in immunological labeling using secondary and tertiary antibodies). The detectable label can be linked by covalent or non-covalent means to the reagent. Alternatively, a detectable label can be linked such as by directly labeling a molecule that achieves binding to the reagent via a ligand-receptor binding pair arrangement or other such specific recognition molecules. Detectable labels can include, but are not limited to radioisotopes, bioluminescent compounds, chromophores, antibodies, chemiluminescent compounds, fluorescent compounds, metal chelates, and enzymes.

In other embodiments, the detection reagent is label with a fluorescent compound. When the fluorescently labeled antibody is exposed to light of the proper wavelength, its presence can then be detected due to fluorescence. In some embodiments, a detectable label can be a fluorescent dye molecule, or fluorophore including, but not limited to fluorescein, phycoerythrin, phycocyanin, o-phthaldehyde, fluorescamine, Cy3™, Cy5™, allophycocyanine, Texas Red, peridenin chlorophyll, cyanine, tandem conjugates such as phycoerythrin-Cy5™, green fluorescent protein, rhodamine, fluorescein isothiocyanate (FITC) and Oregon Green™, rhodamine and derivatives (e.g., Texas red and tetrarhodimine isothiocynate (TRITC)), biotin, phycoerythrin, AMCA, CyDyes™, 6-carboxyfhiorescein (commonly known by the abbreviations FAM and F), 6-carboxy-2′,4′,7′,4,7-hexachlorofiuorescein (HEX), 6-carboxy-4′,5′-dichloro-2′,7′-dimethoxyfluorescein (JOE or J), N,N,N′,N′-tetramethyl-6carboxyrhodamine (TAMRA or T), 6-carboxy-X-rhodamine (ROX or R), 5-carboxyrhodamine-6G (R6G5 or G5), 6-carboxyrhodamine-6G (R6G6 or G6), and rhodamine 110; cyanine dyes, e.g. Cy3, Cy5 and Cy7 dyes; coumarins, e.g., umbelliferone; benzimide dyes, e.g. Hoechst 33258; phenanthridine dyes, e.g. Texas Red; ethidium dyes; acridine dyes; carbazole dyes; phenoxazine dyes; porphyrin dyes; polymethine dyes, e.g. cyanine dyes such as Cy3, Cy5, etc; BODIPY dyes and quinoline dyes. In some embodiments, a detectable label can be a radiolabel including, but not limited to ³H, ¹²⁵I, ³⁵S, ¹⁴C, ³²P, and ³³P. In some embodiments, a detectable label can be an enzyme including, but not limited to horseradish peroxidase and alkaline phosphatase. An enzymatic label can produce, for example, a chemiluminescent signal, a color signal, or a fluorescent signal. Enzymes contemplated for use to detectably label an antibody reagent include, but are not limited to, malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholinesterase. In some embodiments, a detectable label is a chemiluminescent label, including, but not limited to lucigenin, luminol, luciferin, isoluminol, theromatic acridinium ester, imidazole, acridinium salt and oxalate ester. In some embodiments, a detectable label can be a spectral colorimetric label including, but not limited to colloidal gold or colored glass or plastic (e.g., polystyrene, polypropylene, and latex) beads.

In some embodiments, detection reagents can also be labeled with a detectable tag, such as c-Myc, HA, VSV-G, HSV, FLAG, V5, HIS, or biotin. Other detection systems can also be used, for example, a biotin-streptavidin system. In this system, the antibodies immunoreactive (e.g., specific for) with the biomarker of interest is biotinylated. Quantity of biotinylated antibody bound to the biomarker is determined using a streptavidin-peroxidase conjugate and a chromagenic substrate. Such streptavidin peroxidase detection kits are commercially available, e.g., from DAKO™; Carpinteria, Calif. A reagent can also be detectably labeled using fluorescence emitting metals such as ¹⁵²Eu, or others of the lanthanide series. These metals can be attached to the reagent using such metal chelating groups as diethylenetriaminepentaacetic acid (DTPA) or ethylenediaminetetraacetic acid (EDTA).

In some embodiments of any of the aspects described herein, the level of expression products of more than one gene can be determined simultaneously (e.g. a multiplex assay) or in parallel. In some embodiments, the level of expression products of no more than 200 other genes is determined. In some embodiments, the level of expression products of no more than 100 other genes is determined. In some embodiments, the level of expression products of no more than 20 other genes is determined. In some embodiments, the level of expression products of no more than 10 other genes is determined.

The term “sample” or “test sample” as used herein denotes a sample taken or isolated from a biological organism, e.g., a blood sample or tumor sample from a subject. Exemplary biological samples include, but are not limited to, a biofluid sample, whole blood, serum, plasma, urine, saliva, a tumor sample, circulating tumor cells, exosomes, a tumor biopsy, and/or tissue sample etc. The term also includes a mixture of the above-mentioned samples. The term “test sample” also includes untreated or pretreated (or pre-processed) biological samples. In some embodiments, a test sample can comprise cells from subject. In some embodiments, a test sample can be a tumor cell test sample, e.g. the sample can comprise cancerous cells, cells from a tumor, and/or a tumor biopsy. In some embodiments, the test sample can be a urine sample.

The test sample can be obtained by removing a sample of cells from a subject, but can also be accomplished by using previously isolated cells (e.g. isolated at a prior timepoint and isolated by the same or another person). In addition, the test sample can be freshly collected or a previously collected sample. In some embodiments, the test sample can be cells that have been expanded or grown in culture. In other embodiments, the test sample can be cells of a cell line.

In some embodiments, the test sample can be an untreated test sample. As used herein, the phrase “untreated test sample” refers to a test sample that has not had any prior sample pre-treatment except for dilution and/or suspension in a solution. Exemplary methods for treating a test sample include, but are not limited to, centrifugation, filtration, sonication, homogenization, heating, freezing and thawing, and combinations thereof. In some embodiments, the test sample can be a frozen test sample, e.g., a frozen tissue. The frozen sample can be thawed before employing methods, assays and systems described herein. After thawing, a frozen sample can be centrifuged before being subjected to methods, assays and systems described herein. In some embodiments, the test sample is a clarified test sample, for example, by centrifugation and collection of a supernatant comprising the clarified test sample. In some embodiments, a test sample can be a pre-processed test sample, for example, supernatant or filtrate resulting from a treatment selected from the group consisting of centrifugation, filtration, thawing, purification, and any combinations thereof. In some embodiments, the test sample can be treated with a chemical and/or biological reagent. Chemical and/or biological reagents can be employed to protect and/or maintain the stability of the sample, including biomolecules (e.g., nucleic acid and protein) therein, during processing. One exemplary reagent is a protease inhibitor, which is generally used to protect or maintain the stability of protein during processing. The skilled artisan is well aware of methods and processes appropriate for pre-processing of biological samples required for determination of the level of an expression product as described herein.

In some embodiments, the methods, assays, and systems described herein can further comprise a step of obtaining a test sample from a subject. In some embodiments, the subject is a human subject.

### Arrays and Uses Thereof for Measuring Expression of a Combination of Markers

As used herein, the term “gene expression profile” refers to a reference to univariate or multivariate gene expression results. For example, the “profile” can correlate to the expression level of one or more marker genes as described herein or the result of the multivariate analysis of the genes and/or gene sets hereinbefore described.

As used herein, the term “microarray” refers to a linear or multi-dimensional array of preferably discrete regions, each having a defined area, formed on the surface of a solid support. The density of the discrete regions on a microarray is determined by the total numbers of target polynucleotides to be detected on the surface of a single solid phase support, e.g., at least about 50/cm², at least about 100/cm², at least about 500/cm², or at least about 1,000/cm². As used herein, a DNA microarray is an array of oligonucleotide probes placed onto a chip or other surfaces used to amplify or clone target polynucleotides. Since the position of each particular group of probes in the array is typically known, the identities of the target polynucleotides can be determined based on their binding to a particular position in the microarray.

DNA microarray technology can be used to conduct a large scale assay of a plurality of target nucleic acid molecules on a single solid phase support. One of ordinary skill in the art can understand and implement methods for immobilizing an array of oligonucleotide probes for hybridization and detection of specific nucleic acid sequences in a sample. Breifly, target polynucleotides of interest isolated from a tissue of interest are hybridized to the DNA chip and the specific sequences detected based on the target polynucleotides' preference and degree of hybridization at discrete probe locations. One important use of arrays is in the analysis of differential gene expression, where the profile of expression of genes in different cells or tissues, often a tissue of interest and a control tissue, is compared and any differences in gene expression among the respective tissues are identified. Such information is useful for the identification of the types of genes expressed in a particular tissue type and diagnosis of conditions based on the expression profile.

In one embodiment, RNA from the sample of interest is subjected to reverse transcription to obtain labelled cDNA. See U.S. Pat. No. 6,410,229 (Lockhart et al.) The cDNA can then be hybridized to oligonucleotides or cDNAs of known sequence arrayed on a chip or other surface in a known order. In another example, the RNA is isolated from a biological sample and hybridized to a chip on which are anchored cDNA probes. The location of the oligonucleotide to which the labelled cDNA hybridizes provides sequence information on the cDNA, while the amount of labelled hybridized RNA or cDNA provides an estimate of the relative representation of the RNA or cDNA of interest. See Schena, et al. Science 270:467-470 (1995). For example, use of a cDNA microarray to analyze gene expression patterns in human cancer is described by DeRisi, et al. (Nature Genetics 14:457-460 (1996)).

In another embodiment, nucleic acid probes corresponding to the subject nucleic acids (e.g., the markers of any one of Tables 1-4) are made. The nucleic acid probes attached to the biochip are designed to be substantially complementary to the nucleic acids of the biological sample such that specific hybridization of the target sequence and the probes occurs. This complementarity need not be perfect, in that there may be any number of base pair mismatches that will interfere with hybridization between the target sequence and the single stranded nucleic acids of the present invention. It is expected that the overall homology of the genes at the nucleotide level probably will be about 40% or greater, probably about 60% or greater, and even more probably about 80% or greater; and in addition that there will be corresponding contiguous sequences of about 8-12 nucleotides or longer. However, if the number of mutations is so great that no hybridization can occur under even the least stringent of hybridization conditions, the sequence is not a complementary target sequence. Thus, by “substantially complementary” herein is meant that the probes are sufficiently complementary to the target sequences to hybridize under normal reaction conditions, particularly high stringency conditions.

A nucleic acid probe is generally single stranded but can be partly single and partly double stranded. The strandedness of the probe is dictated by the structure, composition, and properties of the target sequence. In general, the oligonucleotide probes range from about 6, 8, 10, 12, 15, 20, 30 to about 100 bases long, with from about 10 to about 80 bases being preferred, and from about 15 to about 40 bases being particularly preferred. That is, generally entire genes are rarely used as probes. In some embodiments, much longer nucleic acids can be used, up to hundreds of bases. The probes are sufficiently specific to hybridize to a complementary template sequence under conditions known by those of skill in the art. The number of mismatches between the probe's sequences and their complementary template (target) sequences to which they hybridize during hybridization generally do not exceed 15%, do not exceed 10% or do not exceed 5%, as-determined by BLAST (default settings).

Oligonucleotide probes can include the naturally-occurring heterocyclic bases normally found in nucleic acids (uracil, cytosine, thymine, adenine and guanine), as well as modified bases and base analogues. Any modified base or base analogue compatible with hybridization of the probe to a target sequence is useful with the methods and assays described herein. The sugar or glycoside portion of the probe can comprise deoxyribose, ribose, and/or modified forms of these sugars, such as, for example, 2′-O-alkyl ribose. In a preferred embodiment, the sugar moiety is 2′-deoxyribose; however, any sugar moiety that is compatible with the ability of the probe to hybridize to a target sequence can be used.

In one embodiment, the nucleoside units of the probe are linked by a phosphodiester backbone, as is well known in the art. In additional embodiments, internucleotide linkages can include any linkage known to one of skill in the art that is compatible with specific hybridization of the probe including, but not limited to phosphorothioate, methylphosphonate, sulfamate (e.g., U.S. Pat. No. 5,470,967) and polyamide (i.e., peptide nucleic acids). Peptide nucleic acids are described in Nielsen et al. (1991) Science 254: 1497-1500, U.S. Pat. No. 5,714,331, and Nielsen (1999) Curr. Opin. Biotechnol. 10:71-75.

In certain embodiments, the probe can be a chimeric molecule; e.g., can comprise more than one type of base or sugar subunit, and/or the linkages can be of more than one type within the same primer. The probe can comprise a moiety to facilitate hybridization to its target sequence, as are known in the art, for example, intercalators and/or minor groove binders. Variations of the bases, sugars, and internucleoside backbone, as well as the presence of any pendant group on the probe, will be compatible with the ability of the probe to bind, in a sequence-specific fashion, with its target sequence. A large number of structural modifications are possible within these bounds. Advantageously, the probes according to the methods and assays described herein can have structural characteristics such that they allow the signal amplification, such structural characteristics being, for example, branched DNA probes as those described by Urdea et al. (Nucleic Acids Symp. Ser., 24:197-200 (1991)). Moreover, synthetic methods for preparing the various heterocyclic bases, sugars, nucleosides and nucleotides that form the probe, and preparation of oligonucleotides of specific predetermined sequence, are well-developed and known in the art.

Multiple probes can be designed for a particular target nucleic acid to account for polymorphism and/or secondary structure in the target nucleic acid, redundancy of data and the like. In some embodiments, where more than one probe per sequence is used, either overlapping probes or probes to different sections of a single target gene are used. That is, two, three, four or more probes, are used to build in a redundancy for a particular target. The probes can be overlapping (e.g., have some sequence in common), or are specific for distinct sequences of a gene. When multiple target polynucleotides are to be detected according to the methods and assays described herein, each probe or probe group corresponding to a particular target polynucleotide is situated in a discrete area of the microarray.

Probes can be in solution, such as in wells or on the surface of a micro-array, or attached to a solid support. Examples of solid support materials that can be used include a plastic, a ceramic, a metal, a resin, a gel and a membrane. Useful types of solid supports include plates, beads, magnetic material, microbeads, hybridization chips, membranes, crystals, ceramics and self-assembling monolayers. One example comprises a two-dimensional or three-dimensional matrix, such as a gel or hybridization chip with multiple probe binding sites (Pevzner et al., J. Biomol. Struc. & Dyn. 9:399-410, 1991; Maskos and Southern, Nuc. Acids Res. 20:1679-84, 1992).

Hybridization chips can be used to construct very large probe arrays that are subsequently hybridized with a target nucleic acid. Analysis of the hybridization pattern of the chip can assist in the identification of the target nucleotide sequence. Patterns can be manually or computer analyzed, but it is clear that positional sequencing by hybridization lends itself to computer analysis and automation. In another example, one may use an AFFYMETRIX™ chip on a solid phase structural support in combination with a fluorescent bead based approach. In yet another example, one may utilize a cDNA microarray. In one embodiment, the oligonucleotides described by Lockkart et al. (e.g., AFFYMETRIX™ synthesis probes in situ on the solid phase) are used, that is, photolithography.

As will be appreciated by those in the art, nucleic acids can be attached or immobilized to a solid support in a wide variety of ways. By “immobilized” herein is meant the association or binding between the nucleic acid probe and the solid support is sufficient to be stable under the conditions of binding, washing, analysis, and removal. The binding can be covalent or non-covalent. By “non-covalent binding” and grammatical equivalents herein is meant one or more of either electrostatic, hydrophilic, and hydrophobic interactions. Included in non-covalent binding is the covalent attachment of a molecule, such as streptavidin, to the support and the non-covalent binding of the biotinylated probe to the streptavidin. By “covalent binding” and grammatical equivalents herein is meant that the two moieties, the solid support and the probe, are attached by at least one bond, including sigma bonds, pi bonds and coordination bonds. Covalent bonds can be formed directly between the probe and the solid support or can be formed by a cross linker or by inclusion of a specific reactive group on either the solid support or the probe or both molecules. Immobilization may also involve a combination of covalent and non-covalent interactions.

Nucleic acid probes can be attached to the solid support by covalent binding such as by conjugation with a coupling agent or by covalent or non-covalent binding such as electrostatic interactions, hydrogen bonds or antibody-antigen coupling, or by combinations thereof. Typical coupling agents include biotin/avidin, biotin/streptavidin, Staphylococcus aureus protein A/IgG antibody Fc fragment, and streptavidin/protein A chimeras (T. Sano and C. R. Cantor, Bio/Technology 9:1378-81 (1991)), or derivatives or combinations of these agents. Nucleic acids may be attached to the solid support by a photocleavable bond, an electrostatic bond, a disulfide bond, a peptide bond, a diester bond or a combination of these sorts of bonds. The arraycan also be attached to the solid support by a selectively releasable bond such as 4,4′-dimethoxytrityl or its derivative. Derivatives which have been found to be useful include 3 or 4[bis-(4-methoxyphenyl)]-methyl-benzoic acid, N-succinimidyl-3 or 4[bis-(4-methoxyphenyl)]-methyl-benzoic acid, N-succinimidyl-3 or 4[bis-(4-methoxyphenyl)]-hydroxymethyl-benzoic acid, N-succinimidyl-3 or 4[bis-(4-methoxyphenyl)]-chloromethyl-benzoic acid, and salts of these acids.

In general, the probes are attached to the biochip in a wide variety of ways, as will be appreciated by those in the art. As described herein, the nucleic acids can either be synthesized first, with subsequent attachment to the biochip, or can be directly synthesized on the biochip.

The biochip comprises a suitable solid substrate. By “substrate” or “solid support” or other grammatical equivalents herein is meant any material that can be modified to contain discrete individual sites appropriate for the attachment or association of the nucleic acid probes and is amenable to at least one detection method. The solid phase support of the array, biochip or microarray can be of any solid materials and structures suitable for supporting nucleotide hybridization and synthesis. Preferably, the solid phase support comprises at least one substantially rigid surface on which the primers can be immobilized and the reverse transcriptase reaction performed. The substrates with which the polynucleotide microarray elements are stably associated and can be fabricated from a variety of materials, including plastics, ceramics, metals, acrylamide, cellulose, nitrocellulose, glass, polystyrene, polyethylene vinyl acetate, polypropylene, polymethacrylate, polyethylene, polyethylene oxide, polysilicates, polycarbonates, TEFLON®, fluorocarbons, nylon, silicon rubber, polyanhydrides, polyglycolic acid, polylactic acid, polyorthoesters, polypropylfumerate, collagen, glycosaminoglycans, and polyamino acids. Substrates can be two-dimensional or three-dimensional in form, such as gels, membranes, thin films, glasses, plates, cylinders, beads, magnetic beads, optical fibers, woven fibers, etc. In one embodiment, the array is a three-dimensional array. In another embodiment, the three-dimensional array is a collection of tagged beads. Each tagged bead has different primers attached to it. Tags are detectable by signalling means such as color (LUMINEX™, ILLUMINA™) and electromagnetic field (PHARMASEQ™) and signals on tagged beads can even be remotely detected (e.g., using optical fibers). The size of the solid support can be any of the standard microarray sizes, useful for DNA microarray technology, and the size can be tailored to fit the particular machine being used to conduct a reaction for use with the methods and assays described herein. In general, the substrates allow optical detection and do not appreciably fluoresce.

In one embodiment, the surface of the biochip and the probe can be derivatized with chemical functional groups for subsequent attachment of the two. Thus, for example, the biochip is derivatized with a chemical functional group including, but not limited to, amino groups, carboxy groups, oxo groups and thiol groups, with amino groups being particularly preferred. Using these functional groups, the probes can be attached using functional groups on the probes. For example, nucleic acids containing amino groups can be attached to surfaces comprising amino groups, for example using linkers as are known in the art; for example, homo- or hetero-bifunctional linkers as are well known. In addition, in some cases, additional linkers, such as alkyl groups (including substituted and heteroalkyl groups) can be used. In this embodiment, the oligonucleotides are synthesized as is known in the art, and then attached to the surface of the solid support. As will be appreciated by those skilled in the art, either the 5′ or 3′ terminus may be attached to the solid support, or attachment may be via an internal nucleoside. In an additional embodiment, the immobilization to the solid support may be very strong, yet non-covalent. For example, biotinylated oligonucleotides can be made, which bind to surfaces covalently coated with streptavidin, resulting in attachment.

The arrays described herein can be produced according to any convenient methodology, such as preforming the polynucleotide microarray elements and then stably associating them with the surface. Alternatively, the oligonucleotides can be synthesized on the surface, as is known in the art. A number of different array configurations and methods for their production are known to those of skill in the art and disclosed in WO 95/25116 and WO 95/35505 (photolithographic techniques), U.S. Pat. No. 5,445,934 (in situ synthesis by photolithography), U.S. Pat. No. 5,384,261 (in situ synthesis by mechanically directed flow paths); and U.S. Pat. No. 5,700,637 (synthesis by spotting, printing or coupling); the disclosure of which are herein incorporated in their entirety by reference. Another method for coupling DNA to beads uses specific ligands attached to the end of the DNA to link to ligand-binding molecules attached to a bead. Possible ligand-binding partner pairs include biotin-avidin/streptavidin, or various antibody/antigen pairs such as digoxygenin-antidigoxygenin antibody (Smith et al., Science 258.1122-1126 (1992)). Covalent chemical attachment of DNA to the support can be accomplished by using standard coupling agents to link the 5′-phosphate on the DNA to coated microspheres through a phosphoamidate bond. Methods for immobilization of oligonucleotides to solid-state substrates are well established. See Pease et al., Proc. Natl. Acad. Sci. USA 91(11):5022-5026 (1994). A preferred method of attaching oligonucleotides to solid-state substrates is described by Guo et al., Nucleic Acids Res. 22:5456-5465 (1994) Immobilization can be accomplished either by in situ DNA synthesis (Maskos and Southern, supra) or by covalent attachment of chemically synthesized oligonucleotides (Guo et al., supra) in combination with robotic arraying technologies.

In addition to the solid-phase technology represented by biochip arrays, gene expression can also be quantified using liquid-phase arrays. One such system is kinetic polymerase chain reaction (PCR). Kinetic PCR allows for the simultaneous amplification and quantification of specific nucleic acid sequences. The specificity is derived from synthetic oligonucleotide primers designed to preferentially adhere to single-stranded nucleic acid sequences bracketing the target site. This pair of oligonucleotide primers form specific, non-covalently bound complexes on each strand of the target sequence. These complexes facilitate in vitro transcription of double-stranded DNA in opposite orientations. Temperature cycling of the reaction mixture creates a continuous cycle of primer binding, transcription, and re-melting of the nucleic acid to individual strands. The result is an exponential increase of the target dsDNA product. This product can be quantified in real time either through the use of an intercalating dye or a sequence specific probe. SYBR(r) Green 1, is an example of an intercalating dye, that preferentially binds to dsDNA resulting in a concomitant increase in the fluorescent signal. Sequence specific probes, such as used with TaqMan® technology, consist of a fluorochrome and a quenching molecule covalently bound to opposite ends of an oligonucleotide. The probe is designed to selectively bind the target DNA sequence between the two primers. When the DNA strands are synthesized during the PCR reaction, the fluorochrome is cleaved from the probe by the exonuclease activity of the polymerase resulting in signal dequenching. The probe signalling method can be more specific than the intercalating dye method, but in each case, signal strength is proportional to the dsDNA product produced. Each type of quantification method can be used in multi-well liquid phase arrays with each well representing primers and/or probes specific to nucleic acid sequences of interest. When used with messenger RNA preparations of tissues or cell lines, an array of probe/primer reactions can simultaneously quantify the expression of multiple gene products of interest. See Germer et al., Genome Res. 10:258-266 (2000); Heid et al., Genome Res. 6:986-994 (1996).

### Normalizing Expression Data to a Control

In some embodiments, the expression levels of each of the markers assayed is normalized to a control. Suitable controls for normalizing expression levels of biomarkers are known to those of skill in the art. In certain embodiments, any gene or gene product can be used as a normalizing control, provided that the mRNA or protein is constitutively expressed, and is not differentially regulated in disease states (e.g., cancer). One of skill in the art can easily determine if a gene or gene product can be used as a normalizing marker by comparing the expression levels in samples taken at different time points from one individual, or among a plurality of samples taken from diseased (e.g., cancer) and control populations. Typically, an appropriate normalization control marker will not fluctuate widely (e.g., less than 30%) among time points or among disease populations when assessed using an assay (e.g., a microarray).

In some embodiments, the control can be the total expression level of the markers assayed, which can include the markers assayed for determining a metagene value and can also include additional control markers. For example, when an array (e.g., a microarray) is used to assay the expression level of the markers e.g., in Tables 1, 2, or 3, the control can be a gene or set of genes on the array. The control can include the markers of Tables 1, 2 or 3, for example, when the exposure of the array is normalized to a desired level. Alternatively, the control can include a specific set of control markers, for example, the markers in Table 4. In some embodiments, the control is the expression level of at least 1 marker listed in Table 4. In other embodiments, the control is the expression level of at least 2, at least at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24 or at least 25 markers listed in Table 4.

TABLE 4
An exemplary gene set useful for normalizingexpression
of a combination of markers

| | Entrez |
|---|---|
| | Gene |
| Official symbol | ID |
| ACTB | 11461 |
| HMBS | 3145 |
| RPLP0 | 6175 |
| UBC | 7316 |
| ZFAND3 | 60685 |
| ZNF212 | 7988 |
| FBXO34 | 55030 |
| DDX3X | 1654 |
| SDHAF1 | 644096 |
| EIF4G2 | 1982 |
| USP12 | 219333 |
| MUS81 | 80198 |
| HDAC6 | 10013 |
| CTDSP2 | 10106 |
| COPS7A | 50813 |
| CTCF | 10664 |
| ANAPC5 | 51433 |
| VPS11 | 55823 |
| DPP8 | 54878 |
| UBAP1 | 51271 |
| HBXIP | 10542 |
| UBA1 | 7317 |
| PRR13 | 54458 |
| TRA2B | 6434 |
| OPA1 | 4976 |
| RAB7A | 7879 |
| MAPRE1 | 22919 |
| RXRB | 6257 |
| SNW1 | 22938 |
| TTC31 | 64427 |
| PUM2 | 23369 |
| C12orf41 | 54934 |
| SAP130 | 79595 |
| BFAR | 51283 |

In embodiments where an array is used to assay the expression level of the combination of markers as described herein, the control is the expression of at least 5 markers on the array; in other embodiments, the control is the expression of at least 10, at least 20, at least 30, at least 40, at least 50, at least 60, at least 70, at least 80, at least 90, at least 100, at least 125, at least 150, at least 200, at least 250, at least 500, at least 1000, at least 2000, at least 3000, at least 4000, at least 5000, at least 7500, at least 10,000, at least 20,000, at least 30,000, at least 40,000 or more markers (e.g., up to and including the total number of markers) on the array.

As used herein, the phrase “normalizing the expression data for each of the markers to a control” or “normalizing” refers to the conversion of a data value representing the level of a marker or a combination of markers as described herein in a sample by dividing it by the expression data value representing the level of a normalizing protein (e.g., actin) or combination of proteins in the sample, thereby permitting comparison of normalized marker values among a plurality of samples, across array platforms, or to a reference.

As used herein, the terms “normalizing protein”, “normalizing factor” and “surrogate marker” are used interchangeably herein and refer to expression of mRNA or protein of a control marker against which the amounts of marker or combination of markers of interest are normalized to permit comparison of amounts of the mRNA or protein of interest among different biological samples. In some embodiments, a normalizing protein is constitutively expressed and is not differentially regulated between at least two physiological states or conditions from which samples will be analyzed, e.g., given disease and non-disease states. Thus, for example, a normalizing control does not vary substantially outside of a range found in a normal healthy population (e.g., <30%, <25%, <20%, <15%, preferably <10%, <7%, <5%, <4%, <3%, <2%, <1% or less) or in the presence and absence of e.g., cancer.

As used herein, the term “housekeeping gene” refers to a gene encoding a protein that is constitutively expressed, and is necessary for basic maintenance and essential cellular functions. A housekeeping gene generally is not expressed in a cell- or tissue-dependent manner, most often being expressed by all cells in a given organism. Some examples of housekeeping proteins include e.g., actin, tubulin, GAPDH, among others. In one embodiment, a housekeeping gene is used as a normalizing protein to which expression of a combination of markers (e.g., from Tables 1, 2, or 3) is compared.

### Calculation of the Metagene Value

The expression level of a combination of markers as described herein can be normalized and analyzed using any number of computations or algorithms (see e.g., U.S. Pat. No. 7,430,475, which is incorporated herein by reference in its entirety). One of ordinary skill in the art of microarray analysis or multiplex gene expression analysis can apply any such algorithm to aid in interpretation of the raw expression data. In one embodiment, an algorithm is generated by “training” it with raw data produced under a variety of conditions. Such trained algorithms will have a coefficient for each gene to improve analysis. However, such training of algorithms is often platform or method specific; that is data cannot be compared across two different platform or methods.

In another embodiment, the algorithm used to analyze raw expression data and calculate a metagene value comprises linear combination. In such embodiments, the coefficient for each gene can be 1. The use of a linear combination analysis permits the resulting metagene value to be compared across different platforms (e.g., microarray platforms). As used herein, the term “linear combination” refers to an expression constructed from a set of terms by multiplying each term by a constant (e.g., 1) and adding the results (e.g., a linear combination of x and y would be any expression of the form ax+by, where a and b are constants). Such methods for calculating a metagene value are known to those of skill in the art.

Alternatively or additionally, a predictive model can be developed and used based on a metagene developed from expression values of two or more genes within a gene group. Models can be developed and used based on selecting the groups as follows, and using one or more of the exemplified genes within the selected groups or a metagene determined from the selected groups, or a gene whose expression is highly correlated with that of an exemplified gene.

Predictions from Tree Models

In one embodiment, analysis of expression values for a combination of markers as described herein comprises averaging the predictions of one or more statistical tree models applied to the metagenes values, wherein each model includes one or more nodes, each node representing a metagene, each node including a statistical predictive probability of tumor recurrence. The statistical tree models may be generated using the methods described herein for the generation of tree models. General methods of generating tree models may also be found in the art (See for example Pitman et al., Biostatistics 2004; 5:587-601; Denison et al. Biometrika 1999; 85:363-77; Nevins et al. Hum Mol Genet 2003; 12:R153-7; Huang et al. Lancet 2003; 361:1590-6; West et al. Proc Natl Acad Sci USA 2001; 98:11462-7; U.S. Patent Pub. Nos. 2003-0224383; 2004-0083084; 2005-0170528; 2004-0106113; and U.S. application Ser. No. 11/198,782).

In one embodiment, the methods and assays described herein comprise deriving a prediction from a single statistical tree model, wherein the model includes one or more nodes, each node representing a metagene, each node including a statistical predictive probability of tumor recurrence. In one embodiment, the tree comprises at least 2 nodes; in other embodiements the tree comprises at least 3 nodes, at least 4 nodes or at least 5 nodes.

In one embodiment, the methods and assays described herein comprise averaging the predictions of one or more statistical tree models applied to the metagenes values, wherein each model includes one or more nodes, each node representing a metagene or a clinical factor, each node including a statistical predictive probability of BRCA1 pathway dysfunction. Accordingly, the methods and assays described herein can use mixed trees, where a tree may contain at least two nodes, where one node represents a metagene and at least one node represents a clinical variable. In one embodiment, the clinical variables can be selected from age of the subject, gender of the subject, tumor size of the sample, stage of cancer disease, histological subtype of the sample and smoking history of the subject, among others.

In one embodiment, the statistical predictive probability is derived from a Bayesian analysis. In another embodiment, the Bayesian analysis includes a sequence of Bayes factor based tests of association to rank and select predictors that define a node binary split, the binary split including a predictor/threshold pair. Bayesian analysis is an approach to statistical analysis that is based on the Bayes law, which states that the posterior probability of a parameter p is proportional to the prior probability of parameter p multiplied by the likelihood of p derived from the data collected. This methodology represents an alternative to the traditional (or frequentist probability) approach: whereas the latter attempts to establish confidence intervals around parameters, and/or falsify a-priori null-hypotheses, the Bayesian approach attempts to keep track of how a-priori expectations about some phenomenon of interest can be refined, and how observed data can be integrated with such a-priori beliefs, to arrive at updated posterior expectations about the phenomenon. Bayesian analysis can be applied to numerous statistical models to predict outcomes of events based on available data. These include standard regression models, e.g. binary regression models, as well as to more complex models that are applicable to multi-variate and essentially non-linear data.

Another such model is commonly known as the tree model which is essentially based on a decision tree. Decision trees can be used in clarification, prediction and regression. A decision tree model is built starting with a root mode, and training data partitioned to what are essentially the “children” nodes using a splitting rule. For instance, for clarification, training data contains sample vectors that have one or more measurement variables and one variable that determines that class of the sample. Various splitting rules may be used; however, the success of the predictive ability varies considerably as data sets become larger. Furthermore, past attempts at determining the best splitting for each mode is often based on a “purity” function calculated from the data, where the data is considered pure when it contains data samples only from one clan. Most frequently used purity functions are entropy, gini-index, and towing rule. A statistical predictive tree model to which Bayesian analysis is applied may consistently deliver accurate results with high predictive capabilities.

### Reference Value

The terms “reference value,” “reference level,” “reference sample,” and “reference” are used interchangeably herein and refer to the level of expression of a control marker or combination of control markers in a known sample against which another sample (i.e., one obtained from a subject having, or suspected of having, cancer) is compared. A reference value is useful for determining the amount of expression of a combination of markers or the relative increase/decrease of such expressional levels in a biological sample. A reference value serves as a reference level for comparison, such that samples can be normalized to an appropriate standard in order to infer the presence, absence or extent of BRCA1 pathway function activity or PARP/DNA damage-inducing chemotherapeutic sensitivity in a subject.

In one embodiment, a biological standard is obtained at an earlier time point (e.g., prior to the onset of a cancer) from the same individual that is to be tested or treated as described herein. Alternatively, a standard can be from the same individual having been taken at a time after the onset or diagnosis of a cancer. In such instances, the reference value can provide a measure of the efficacy of treatment. It can be useful to use as a reference for a given patient a level from a sample taken after a cancer diagnosis but before the administration of any therapy to that patient.

Alternatively, a reference value can be obtained, for example, from a known biological sample from a different individual (e.g., not the individual being tested) that is e.g., substantially free of detectable cancer. A known sample can also be obtained by pooling samples from a plurality of individuals to produce a reference value or range of values over an averaged population, wherein a reference value represents an average level of expression of a combination of markers as described herein among a population of individuals (e.g., a population of individuals substantially free of detectable cancer). Thus, the metagene value or expression level of a combination of control markers in a reference value obtained in this manner is representative of an average level of this marker or combination of markers in a general population of individuals lacking cancer. An individual sample is compared to this population reference value by comparing expression of the same or substantially similar combination of markers from a sample relative to the population reference value. Generally, an increase in the amount of expression over the reference value (e.g., a reference obtained from subjects lacking cancer) indicates low BRCA1 pathway function and/or predicts the sensitivity of a caner to combination treatment as described herein, while a decrease in the amount of expression indicates normal BRCA1 pathway function and/or predicts that the cancer is not as sensitive to the combination treatment described herein. The converse is contemplated in cases where a reference value is obtained from a population of subjects having cancer. It should be noted that there is often variability among individuals in a population, such that some individuals will have higher levels of expression, while other individuals have lower levels of expression. However, one skilled in the art can make logical inferences on an individual basis regarding the detection and treatment of cancer as described herein.

In one embodiment, a range of metagene values or expression levels of a combination of markers can be defined for a plurality of cancer-free subjects and/or for a plurality of subjects having cancer. Provided that the number of individuals in each group is sufficient, one can define a range of metagene or expression values for each population. These values can be used to define cut-off points for selecting a therapy or for monitoring progression of disease. Thus, one of skill in the art can determine a metagene value and compare the value to the ranges in each particular sub-population to aid in determining the status of disease and the recommended course of treatment. Such value ranges are analogous to e.g., HDL and LDL cholesterol levels detected clinically. For example, LDL levels below 100 mg/dL are considered optimal and do not require therapeutic intervention, while LDL levels above 190 mg/dL are considered ‘very high’ and will likely require some intervention. One of skill in the art can readily define similar parameters for metagene values or expression values in a cancer. These value ranges can be provided to clinicians, for example, on a chart, programmed into a PDA etc.

A standard comprising a reference value or range of values can also be synthesized. A known amount of a marker or combination of markers (or a series of known amounts) can be prepared within the typical expression range that is observed in a general cancer or cancer-free population. This method has an advantage of being able to compare the extent of disease in one or more individuals in a mixed population. This method can also be useful for subjects who lack a prior sample to act as a reference value or for routine follow-up post-diagnosis. This type of method can also allow standardized tests to be performed among several clinics, institutions, or countries etc.

### PARP Inhibitors

Poly(ADP-ribose)polymerase (PARP) has an essential role in facilitating DNA repair, controlling RNA transcription, mediating cell death, and regulating immune response. PARP inhibitors have demonstrated efficacy in numerous models of disease, particularly in models of ischemia reperfusion injury, inflammatory disease, degenerative diseases, protection from adverse effects of cytoxic compounds, and the potentiation of cytotoxic cancer therapy.

PARP1 is a protein that is important for repairing single-strand breaks (‘nicks’ in the DNA). If such nicks persist unrepaired until DNA is replicated (which must precede cell division), then the replication itself can cause double strand breaks to form. Drugs that inhibit PARP1 cause multiple double strand breaks to form in this way, and in tumors with BRCA1, BRCA2 or PALB2 mutations these double strand breaks cannot be efficiently repaired, leading to the death of the cells. Normal cells that don't replicate their DNA as often as cancer cells, and that lack any mutated BRCA1 or BRCA2 still have homologous repair operating, which allows them to survive the inhibition of PARP.

Thus, a PARP inhibitor can be used in combination with a DNA damage-inducing chemotherapeutic to treat a cancer or cancer cell having reduced BRCA1 pathway function in accordance with the methods and assays described herein. Any PARP inhibitor can be used including, but not limited to, olaparib (AZD-2281), rucaparib (AG 014669), veliparib (ABT-888), CEP-9722, MK-4827, BMN-673, 3-aminobenzamide, 5-aminoisoquinolinone (AIQ), and PJ-34. Other PARP inhibitors may also be used with the methods and assays described herein. For example, the following PARP inhibitors are commercially available from ALEXIS BIOCHEMICALS™: 1,5-Isoquinolinediol; 3-Methyl-5-AIQ hydrochloride; 4-Amino-1,8-naphthalimide; 4-Hydroxyquinazoline; 5-AIQ hydrochloride; 5-Iodo-6-amino-1,2-benzopyrone; 6(5H)-Phenanthridinone; EB-47 dihydrochloride dihydrate; NU1025; TIQ-A; DR2313; PJ-34.

Additional PARP inhibitors and their sources also include: BSI 401 (BIPAR SCIENCES™); BSI 201 (BIPAR SCIENCES™); AZD 2281 (KU-0059436) (KUDOS PHARMACEUTICALS™); INO 1001 (INOTEK PHARMACEUTICALS™); GPI 15427 (10-(4-methyl-piperazin-1-ylmethyl)-2H-7-oxa-1,2-diaza-benzo[de] anthracen-3-one) (Salvatore Cuzzocrea et al.); GPI 16539 (2-(4-methyl-piperazin-1-yl)-5H-benzo[c][1,5]naphthyridin-6-one) (Salvatore Cuzzocrea et al.); GPI 6150 (1,11b-dihydro-[2H]benzopyrano[4,3,2-de]isoquinolin-3-one) (Salvatore Cuzzocrea et al.); DR2313 (CALBIOCHEM™, ALEXIS BIOCHEMICALS™); AG14361 (PFIZER™); NU1025 (8-hydroxy-2 methyl-quinazolin-4-[3H]one) (ALEXIS BIOCHEMICALS™);); CEP 6800 (CEPHALON™, Pa., USA); AG 014699 (developed by collaboration among Newcastle University, Cancer Research UK, and Agouron Pharmaceuticals); ABT-888 ((2-[(R)-2-methylpyrrolidin-2-yl]-1H-benzimidazole-4-carboxamide) (ABBOTT LABORATORIES™); minocycline or other tetracycline derivatives (SIGMA-ALDRICH™, ALEXIS BIOCHEMICALS™, among others).

### DNA Damage-Inducing Chemotherapeutics

Essentially any chemotherapeutic comprising a mechanism of DNA damage can be used with a PARP inhibitor in accordance with the methods and assays described herein. In one embodiment, the DNA damage-inducing chemotherapeutic is a DNA alkylating agent.

A DNA alkylating agent is an alkylating agent used in cancer treatment that attaches an alkyl group to DNA. Since cancer cells, in general, proliferate faster and with less error-correcting than healthy cells, cancer cells are more sensitive to DNA damage, including alkylation. Exemplary DNA alkylating agents for use with the methods and assays described herein include, but are not limited to, cyclophosphamide, mechloroethamine, uramustine, melphalan, chlorambucil, ifosfamide, bendamustine, carmustine, lomustine, streptozocin, busulfan, cisplatin, carboplatin, nedaplatin, oxaliplatin, satraplatin, triplatin tetranitrate, procarbazine, altretamine, and tetrazines (e.g., dacarbazine, mitozolomide, temozolomide etc.).

### Treatment of Cancer

As used herein, the terms “treat” “treatment,” “treating,” or “amelioration” refer to therapeutic treatments, wherein the object is to reverse, alleviate, ameliorate, inhibit, slow down or stop the progression or severity of a condition associated with a disease or disorder. The term “treating” includes reducing or alleviating at least one adverse effect or symptom of a condition, disease or disorder associated with cancer. Treatment is generally “effective” if one or more symptoms or clinical markers are reduced. Alternatively, treatment is “effective” if the progression of a disease is reduced or halted. That is, “treatment” includes not just the improvement of symptoms or markers, but can also include a cessation or at least slowing of progress or worsening of symptoms that would be expected in absence of treatment. Beneficial or desired clinical results include, but are not limited to, alleviation of one or more symptom(s) of a cancer (e.g., tumor size), diminishment of extent of a cancer or tumor, stabilized (i.e., not worsening) cancer, delay or slowing of progression of the disease, amelioration or palliation of the disease state, and remission (whether partial or total). The term “treatment” of a disease also includes providing at least partial relief from the symptoms or side-effects of the disease (including palliative treatment). Treatment does not encompass cure.

In one embodiment, as used herein, the term “prevention” or “preventing” when used in the context of a subject refers to stopping, hindering, and/or slowing the development of a cancer or metastasis from a primary or a secondary tumor.

As used herein, the term “therapeutically effective amount” means that amount necessary, at least partly, to attain the desired effect, or to delay the onset of, inhibit the progression of, or halt altogether, the onset or progression of the particular disease or disorder being treated (e.g., cancer). Such amounts will depend, of course, on the particular condition being treated, the severity of the condition and individual patient parameters including age, physical condition, size, weight and concurrent treatment. These factors are well known to those of ordinary skill in the art and can be addressed with no more than routine experimentation. In some embodiments, a maximum dose of a therapeutic agent is used, that is, the highest safe dose according to sound medical judgment. It will be understood by those of ordinary skill in the art, however, that a lower dose or tolerable dose that is effective can be administered for medical reasons, psychological reasons or for virtually any other reason.

In one embodiment, a therapeutically effective amount of a pharmaceutical formulation, or a composition described herein for a method of treating cancer is an amount sufficient to reduce the level of at least one symptom of cancer (e.g., tumor size, tumor growth rate, number of circulating tumor cells etc.) as compared to the level in the absence of the compound, the combination of compounds, the pharmaceutical composition/formulation of the composition. In other embodiments, the amount of the composition administered is preferably safe and sufficient to treat, delay the development of cancer, and/or delay onset of the disease. In some embodiments, the amount can thus cure or result in amelioration of the symptoms of cancer, slow the course of the disease, slow or inhibit a symptom of the disease, or slow or inhibit the establishment or development of secondary symptoms of a cancer. As but two examples, an effective amount of a composition described herein can inhibit further symptoms associated with a cancer or cause a reduction in one or more symptoms associated with a cancer. While effective treatment need not necessarily initiate complete regression of the disease, such effect would be effective treatment. The effective amount of a given therapeutic agent will vary with factors such as the nature of the agent, the route of administration, the size and species of the animal to receive the therapeutic agent, and the purpose of the administration. Thus, it is not possible or prudent to specify an exact “therapeutically effective amount.” However, for any given case, an appropriate “effective amount” can be determined by a skilled artisan according to established methods in the art using only routine experimentation.

### Pharmaceutical Compositions

Provided herein are compositions that are useful for treating a cancer having reduced BRCA1 pathway function in a subject. In one embodiment, the composition is a pharmaceutical composition. The composition can comprise a therapeutically effective amount of a PARP inhibitor in combination with a therapeutically effective amount of a DNA damage-inducing chemotherapeutic.

The composition can optionally include a carrier, such as a pharmaceutically acceptable carrier. Pharmaceutically acceptable carriers are determined in part by the particular composition being administered, as well as by the particular method used to administer the composition. Accordingly, there is a wide variety of suitable formulations of pharmaceutical compositions. Formulations suitable for parenteral administration can be formulated, for example, for intravenous, intramuscular, intradermal, intraperitoneal, and subcutaneous routes. Carriers can include aqueous isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, preservatives, liposomes, microspheres and emulsions.

Therapeutic compositions contain a physiologically tolerable carrier together with an active agent as described herein, dissolved or dispersed therein as an active ingredient. As used herein, the terms “pharmaceutically acceptable”, “physiologically tolerable” and grammatical variations thereof, as they refer to compositions, carriers, diluents and reagents, are used interchangeably and represent that the materials are capable of administration to or upon a mammal without the production of undesirable physiological effects such as nausea, dizziness, gastric upset and the like. A pharmaceutically acceptable carrier will not promote the raising of an immune response to an agent with which it is admixed, unless so desired. The preparation of a pharmaceutical composition that contains active ingredients dissolved or dispersed therein is well understood in the art and need not be limited based on formulation. Typically such compositions are prepared as injectable either as liquid solutions or suspensions; however, solid forms suitable for solution, or suspensions in liquid prior to use can also be prepared. The preparation can also be emulsified or presented as a liposome composition. The active ingredient can be mixed with excipients which are pharmaceutically acceptable and compatible with the active ingredient and in amounts suitable for use in the therapeutic methods described herein. Suitable excipients include, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient. The therapeutic composition of the present invention can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Examples of such additional liquid phases are glycerin, vegetable oils such as cottonseed oil, and water-oil emulsions. The amount of an active agent used in the methods described herein that will be effective in the treatment of a particular disorder or condition will depend on the nature of the disorder or condition, and can be determined by standard clinical techniques.

While any suitable carrier known to those of ordinary skill in the art can be employed in the pharmaceutical compositions of this invention, the type of carrier will vary depending on the mode of administration. Compositions of the present invention can be formulated for any appropriate manner of administration, including for example, topical, oral, nasal, intravenous, intracranial, intraperitoneal, subcutaneous or intramuscular administration. For parenteral administration, such as intramuscular or subcutaneous injection, the carrier preferably comprises water, saline, alcohol, a fat, a wax or a buffer. For oral administration, any of the above carriers or a solid carrier, such as mannitol, lactose, starch, magnesium stearate, sodium saccharine, talcum, cellulose, glucose, sucrose, and magnesium carbonate, may be employed. Biodegradable microspheres (e.g., polylactate polyglycolate) can also be employed as carriers for the pharmaceutical compositions. Suitable biodegradable microspheres are disclosed, for example, in U.S. Pat. Nos. 4,897,268 and 5,075,109. Such compositions can also comprise buffers (e.g., neutral buffered saline or phosphate buffered saline), carbohydrates (e.g., glucose, mannose, sucrose or dextrans), mannitol, proteins, polypeptides or amino acids such as glycine, antioxidants, chelating agents such as EDTA or glutathione, adjuvants (e.g., aluminum hydroxide) and/or preservatives. Alternatively, compositions as described herein can be formulated as a lyophilizate. Compounds can also be encapsulated within liposomes using well known technology. The compositions described herein can be administered as part of a sustained release formulation (i.e., a formulation such as a capsule or sponge that affects a slow release of compound following administration). Such formulations can generally be prepared using well known technology and administered by, for example, oral, rectal or subcutaneous implantation, or by implantation at the desired target site. Sustained-release formulations can contain a polypeptide, polynucleotide dispersed in a carrier matrix and/or contained within a reservoir surrounded by a rate controlling membrane. Carriers for use within such formulations are biocompatible, and can also be biodegradable; preferably the formulation provides a relatively constant level of active component release. The amount of active compound contained within a sustained release formulation depends upon the site of implantation, the rate and expected duration of release and the nature of the condition to be treated or prevented.

### Dosage and Administration

Treatment includes prophylaxis and therapy. Prophylaxis or treatment can be accomplished by a single direct injection at a single time point or multiple time points. Administration can also be nearly simultaneous to multiple sites. Patients or subjects include mammals, such as human, bovine, equine, canine, feline, porcine, and ovine animals as well as other veterinary subjects. Preferably, the patients or subjects are human.

In one aspect, the methods described herein provide a method for treating cancer in a subject (e.g., a cancer associated with reduced BRCA1 pathway function). In one embodiment, the subject can be a mammal. In another embodiment, the mammal can be a human, although the approach is effective with respect to all mammals. The method comprises administering to the subject an effective amount of a pharmaceutical composition comprising a PARP inhibitor in combination with a DNA damage-inducing chemotherapeutic.

The dosage range for the agent depends upon the potency, and includes amounts large enough to produce the desired effect, e.g., reduction in at least one symptom of cancer. The dosage should not be so large as to cause unacceptable adverse side effects. Generally, the dosage will vary with the type of inhibitor (e.g., an antibody or fragment, small molecule, siRNA, etc.) and with the age, condition, and sex of the patient. The dosage can be determined by one of skill in the art and can also be adjusted by the individual physician in the event of any complication. Typically, the dosage ranges from 0.001 mg/kg body weight to 5 g/kg body weight. In some embodiments, the dosage range is from 0.001 mg/kg body weight to 1 g/kg body weight, from 0.001 mg/kg body weight to 0.5 g/kg body weight, from 0.001 mg/kg body weight to 0.1 g/kg body weight, from 0.001 mg/kg body weight to 50 mg/kg body weight, from 0.001 mg/kg body weight to 25 mg/kg body weight, from 0.001 mg/kg body weight to 10 mg/kg body weight, from 0.001 mg/kg body weight to 5 mg/kg body weight, from 0.001 mg/kg body weight to 1 mg/kg body weight, from 0.001 mg/kg body weight to 0.1 mg/kg body weight, from 0.001 mg/kg body weight to 0.005 mg/kg body weight. Alternatively, in some embodiments the dosage range is from 0.1 g/kg body weight to 5 g/kg body weight, from 0.5 g/kg body weight to 5 g/kg body weight, from 1 g/kg body weight to 5 g/kg body weight, from 1.5 g/kg body weight to 5 g/kg body weight, from 2 g/kg body weight to 5 g/kg body weight, from 2.5 g/kg body weight to 5 g/kg body weight, from 3 g/kg body weight to 5 g/kg body weight, from 3.5 g/kg body weight to 5 g/kg body weight, from 4 g/kg body weight to 5 g/kg body weight, from 4.5 g/kg body weight to 5 g/kg body weight, from 4.8 g/kg body weight to 5 g/kg body weight. In one embodiment, the dose range is from 5 μg/kg body weight to 30 μg/kg body weight. Alternatively, the dose range will be titrated to maintain serum levels between 5 μg/mL and 30 μg/mL.

Administration of the doses recited above can be repeated for a limited period of time. In some embodiments, the doses are given once a day, or multiple times a day, for example but not limited to three times a day. In another embodiment, the doses recited above are administered daily for several weeks or months. The duration of treatment depends upon the subject's clinical progress and responsiveness to therapy. Continuous, relatively low maintenance doses are contemplated after an initial higher therapeutic dose.

A therapeutically effective amount is an amount of an agent that is sufficient to produce a statistically significant, measurable change in at least one symptom of a cancer (see “Efficacy Measurement” below). Such effective amounts can be gauged in clinical trials as well as animal studies for a given agent.

Agents useful in the methods and compositions described herein can be administered systemically or can be administered orally. It is also contemplated herein that the agents can also be delivered intravenously (by bolus or continuous infusion), by inhalation, intranasally, intraperitoneally, intramuscularly, subcutaneously, intracavity, and can be delivered by peristaltic means, if desired, or by other means known by those skilled in the art.

In some embodiments, the pharmaceutically acceptable formulation used to administer the active compound provides sustained delivery, such as “slow release” of the active compound to a subject. For example, the formulation can deliver the agent or composition for at least one, two, three, or four weeks after the pharmaceutically acceptable formulation is administered to the subject. Preferably, a subject to be treated in accordance with the methods described herein is treated with the active composition for at least 30 days (either by repeated administration or by use of a sustained delivery system, or both).

As used herein, the term “sustained delivery” is intended to include continual delivery of the composition in vivo over a period of time following administration, preferably at least several days, a week, several weeks, one month or longer. Sustained delivery of the active compound can be demonstrated by, for example, the continued therapeutic effect of the composition over time (such as sustained delivery of the agents can be demonstrated by continued improvement or maintained improvement in cancer symptoms in a subject).

Preferred approaches for sustained delivery include use of a polymeric capsule, a minipump to deliver the formulation, a biodegradable implant, or implanted transgenic autologous cells (as described in U.S. Pat. No. 6,214,622). Implantable infusion pump systems (such as Infusaid; see such as Zierski, J. et al, 1988; Kanoff, R. B., 1994) and osmotic pumps (sold by ALZA CORPORATION) are available in the art. Another mode of administration is via an implantable, externally programmable infusion pump. Suitable infusion pump systems and reservoir systems are described in U.S. Pat. No. 5,368,562 by Blomquist and U.S. Pat. No. 4,731,058 by Doan, developed by Pharmacia Deltec Inc.

Therapeutic compositions containing at least one agent can be conventionally administered in a unit dose. The term “unit dose” when used in reference to a therapeutic composition refers to physically discrete units suitable as unitary dosage for the subject, each unit containing a predetermined quantity of active material calculated to produce the desired therapeutic effect in association with the required physiologically acceptable diluent, i.e., carrier, or vehicle.

The compositions are administered in a manner compatible with the dosage formulation, and in a therapeutically effective amount. The quantity to be administered and timing depends on the subject to be treated, capacity of the subject's system to utilize the active ingredient, and degree of therapeutic effect desired. An agent can be targeted by means of a targeting moiety, such as e.g., an antibody or targeted liposome technology. In some embodiments, an agent can be targeted to a tissue by using bispecific antibodies, for example produced by chemical linkage of an anti-ligand antibody (Ab) and an Ab directed toward a specific target. To avoid the limitations of chemical conjugates, molecular conjugates of antibodies can be used for production of recombinant bispecific single-chain Abs directing ligands and/or chimeric inhibitors at cell surface molecules. The addition of an antibody to an agent permits the agent to accumulate additively at the desired target site (e.g., tumor site). Antibody-based or non-antibody-based targeting moieties can be employed to deliver a ligand or the inhibitor to a target site. Preferably, a natural binding agent for an unregulated or disease associated antigen is used for this purpose.

Precise amounts of active ingredient required to be administered depend on the judgment of the practitioner and are particular to each individual. However, suitable dosage ranges for systemic application are disclosed herein and depend on the route of administration. Suitable regimes for administration are also variable, but are typified by an initial administration followed by repeated doses at one or more intervals by a subsequent injection or other administration. Alternatively, continuous intravenous infusion sufficient to maintain concentrations in the blood or skeletal muscle tissue in the ranges specified for in vivo therapies are contemplated.

### Efficacy Measurement

The efficacy of a given treatment for a cancer as described herein can be determined by the skilled clinician. However, a treatment is considered “effective treatment,” as the term is used herein, if any one or all of the signs or symptoms of a cancer is/are altered in a beneficial manner (e.g., improved strength, reduced muscle atrophy etc.), other clinically accepted symptoms or markers of disease are improved, or even ameliorated, e.g., by at least 10% following treatment with an agent comprising a PARP inhibitor in combination with a DNA damage-inducing chemotherapeutic. Efficacy can also be measured by failure of an individual to worsen as assessed by stabilization of the cancer, hospitalization or need for medical interventions (i.e., progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (1) inhibiting the disease, e.g., arresting, or slowing progression of a cancer; or (2) relieving the disease, e.g., causing regression of symptoms; and (3) preventing or reducing the likelihood of the development of a cancer (e.g., cancer metastases).

An effective amount for the treatment of a disease means that amount which, when administered to a mammal in need thereof, is sufficient to result in effective treatment as that term is defined herein, for that disease. Efficacy of an agent can be determined by assessing physical indicators of a cancer, such as e.g., reduced tumor size, slowed tumor growth rate, reduced angiogenesis etc.

### Companion Diagnostics

The invention also provides a companion diagnostic for methods for treating cancer in a subject. The invention provides a method comprising: administering to a subject a therapeutically effective amount of a poly ADP ribose polymerase (PARP) inhibitor and a DNA damage-inducing chemotherapeutic agent when the subject has been determined to have increased expression of a metagene comprising a combination of markers selected from the group consisting of EZR, SOX4, EMG1, NARS, SNRNP70, CLTA, EIF3B, EIF3E, UBAP2L, SF3A3, HK1, CKAP4, NOLC1, HCFC1, PPM1F, BMS1, AIMP2, POLG, MCM2, U2AF1, MOGS, SKIV2L, CCND1, MLEC, RARS, SLC7A5, MANF, RAE1, CHD4, BYSL, GTF3C2, TRAP1, KIAA0114, CDC25B, SF3B2, SMARCA4, DDX11, LMNB2, DHCR24, SAFB, CTPS, DUSP1, DDX10, CDC123, NDRG1, EIF2B2, FAM38A, SEC24C, RABEPK, FGFR3, SSRP1, and SLC1A3 as compared to a reference value, and not administering a PARP inhibitor and a DNA damage-inducing chemotherapeutic agent when the subject has been determined to have unchanged or decreased expression of a metagene comprising a combination of markers selected from the group consisting of EZR, SOX4, EMG1, NARS, SNRNP70, CLTA, EIF3B, EIF3E, UBAP2L, SF3A3, HK1, CKAP4, NOLC1, HCFC1, PPM1F, BMS1, AIMP2, POLG, MCM2, U2AF1, MOGS, SKIV2L, CCND1, MLEC, RARS, SLC7A5, MANF, RAE1, CHD4, BYSL, GTF3C2, TRAP1, KIAA0114, CDC25B, SF3B2, SMARCA4, DDX11, LMNB2, DHCR24, SAFB, CTPS, DUSP1, DDX10, CDC123, NDRG1, EIF2B2, FAM38A, SEC24C, RABEPK, FGFR3, SSRP1, and SLC1A3 as compared to a reference value.

The method can further comprise a step of assaying for the metagene value and optionally also obtaining a biological cancer-cell comprising sample from the subject. The assaying may comprise expression analysis, normalizing the results of the expression analysis and calculation of the metagene value as taught herein and exemplified below.

Therefore, the invention provides a combination treatment with PARP inhibitor and a DNA damage-inducing chemotherapeutic agent, in a patient indicated to benefit from the treatment based on the metagene value that we have identified as a novel surrogate for analysis of BRCA1 function, wherein when the BRCA1 function is reduced as indicated by the metagene value, the combination treatment is administered based on the expectation that the patient will benefit from the combination treatment and that the side effects are outweight by the benefits, and wherein the BRCA1 function is not reduced, the combination treatment is not administered based on the expectation that it will not benefit the patient and rather is expected to only cause side effects.

The present invention may be as defined in any one of the following numbered paragraphs 1-65.

1. A method of treating cancer in a subject, the method comprising:

(a) assaying a biological sample comprising a cancer cell obtained from a subject having cancer or suspected of having cancer for the expression of a combination of at least the following markers: EZR, SOX4, EMG1, NARS, SNRNP70, CLTA, EIF3B, EIF3E, UBAP2L, SF3A3, HK1, CKAP4, NOLC1, HCFC1, PPM1F, BMS1, AIMP2, POLG, MCM2, U2AF1, MOGS, SKIV2L, CCND1, MLEC, RARS, SLC7A5, MANF, RAE1, CHD4, BYSL, GTF3C2, TRAP1, KIAA0114, CDC25B, SF3B2, SMARCA4, DDX11, LMNB2, DHCR24, SAFB, CTPS, DUSP1, DDX10, CDC123, NDRG1, EIF2B2, FAM38A, SEC24C, RABEPK, FGFR3, SSRP1, and SLC1A3;

(b) normalizing the expression data for each of the markers to a control, and

(c) administering to the subject a therapeutically effective amount of a poly ADP ribose polymerase (PARP) inhibitor and a DNA damage-inducing chemotherapeutic agent when expression of the combination of markers is increased compared to a reference value and not administering a PARP inhibitor and a DNA damage-inducing chemotherapeutic agent when the expression of the combination of markers is not changed or decreased compared to a reference value.

2. A method of determining a BRCA1 functional pathway-deficient cancer in a subject, the method comprising:

(a) assaying a biological sample comprising a cancer cell obtained from a subject having cancer or suspected of having cancer for the expression of a combination of at least the following markers: EZR, SOX4, EMG1, NARS, SNRNP70, CLTA, EIF3B, EIF3E, UBAP2L, SF3A3, HK1, CKAP4, NOLC1, HCFC1, PPM1F, BMS1, AIMP2, POLG, MCM2, U2AF1, MOGS, SKIV2L, CCND1, MLEC, RARS, SLC7A5, MANF, RAE1, CHD4, BYSL, GTF3C2, TRAP1, KIAA0114, CDC25B, SF3B2, SMARCA4, DDX11, LMNB2, DHCR24, SAFB, CTPS, DUSP1, DDX10, CDC123, NDRG1, EIF2B2, FAM38A, SEC24C, RABEPK, FGFR3, SSRP1, and SLC1A3; and

wherein if expression of the combination of markers is increased compared to a reference value the subject is determined to have a BRCA1 functional pathway deficient cancer, and

wherein if the expression of the combination of markers is not changed or reduced compared to the reference value, the subject is determined not to have a BRCA1 functional pathway deficient cancer.

3. The method of paragraph 1 or 2, further comprising assaying the biological sample for at least one additional marker selected from the group consisting of: GCSH, BOP1, XRCC5, CCDC85B, H1FX, FDFT1, DNAJC7, PRKACA, MAP2K5, SNRPA1, RELA, RXRA, KDM5C, PWP2, NME3, FARSA, DNTTIP2, MRPS12, SCRIB, PPAT, CDC37, SLC29A1, SFRS11, SMAD6, EIF4H, VDAC1, NMT1, POLRMT, POLD1, CHAF1A, TAF1C, TCOF1, ALG3, CAD, RASSF7, MTHFD1, CCNE1, HSF1, PSMD1, AGPAT2, PHKG2, PKN1, GAL, SCAP, EIF4G1, DDT, PMPCA, RRP1B, FKBP4, NUP214, ATIC, FASN, AP3D1, RPIA, NSDHL, BRD2, PDCD11, SON, HSP90B1, and ANXA11.
4. The method of paragraph 1, 2 or 3, wherein the control is the expression level of the total set of markers assayed.
5. The method of any one of the preceding paragraphs, further comprising a step of calculating a metagene value to represent the normalized expression of the combination of markers.
6. The method of any one of the preceding paragraphs, wherein the step of calculating the metagene comprises linear combination of the expression values of each marker in the combination of markers.
7. The method of paragraph 6, wherein the linear combination has a coefficient of 1 for each marker.
8. The method of any one of the preceding paragraphs, wherein the biological sample comprises a tumor sample.
9. The method of any one of the preceding paragraphs, wherein the biological sample is assayed for expression of all of the markers SFRS11, EZR, PKN1, SOX4, EMG1, RELA, AIMP2, NARS, HSP90B1, SNRP70, NME3, CLTA, CAD, EIF3B, EIF3E, UBAP2L, SF3A3, HK1, TCOF1, CKAP4, CCNE1, NOLC1, HCFC1, PPM1F, BMS1L, RASSF7, FDFT1, FARSLA, POLG, MCM2, VDAC1, U2AF1, MOGS, ATIC, CHAF1A, SKIV2L, GCSH, GAL, CCDC85B, CCND1, MLEC, RARS, WBSCR1, FKBP4, SLC7A5, MTHFD1, NSDHL, PWP2H, FASN, MANF, EIF4G1, JARID1C, SCRIB, SCAP, PSMD1, RAE1, CHD4, SLC29A1, BYSL, GTF3C2, TRAP1, PRKACA, KIAA0179, KIAA0114, POLD1, DNTTIP2, BOP1, CDC25B, MRPS12, SF3B2, NUP214, PHKG2, AGPAT2, SMARCA4, PPAT, SNRPA1, DDX11, RPIA, ALG3, LMNB2, TAF1C, DDT, DHCR24, SAFB, CDC37, H1FX, AP3D1, DNAJC7, HSF1, CTPS, DUSP1, DDX10, PDCD11, CDC123, RXRA, NDRG1, EIF2B2, ANXA11, FAM38A, XRCC5, SEC24C, PMPCA, POLRMT, RABEPK, SMAD6, NMT1, SON, MAP2K5, SLC1A3, FGFR3, SSRP1, and BRD2.
10. The method of any one of the preceding paragraphs, wherein the subject is known to carry a mutation in the BRCA1 gene.
11. The method of any one of the preceding paragraphs, wherein the subject is known to carry a wild-type BRCA1 gene.
12. The method of any one of the preceding paragraphs, wherein the BRCA1 status of the subject is unknown.
13. The method of any one of the preceding paragraphs, wherein the cancer is breast cancer.
14. The method of any one of the preceding paragraphs, wherein the expression assayed is mRNA expression.
15. The method of any one of the preceding paragraphs, wherein the expression assayed is protein expression.
16. A method for selecting a PARP inhibitor/DNA damaging chemotherapeutic-sensitive or PARP inhibitor/DNA damaging chemotherapeutic-insensitive subject having cancer, the method comprising:
(a) assaying a biological sample comprising a cancer cell obtained from a subject having cancer or suspected of having cancer for the expression of a combination of at least the following markers: EZR, SOX4, EMG1, NARS, SNRNP70, CLTA, EIF3B, EIF3E, UBAP2L, SF3A3, HK1, CKAP4, NOLC1, HCFC1, PPM1F, BMS1, AIMP2, POLG, MCM2, U2AF1, MOGS, SKIV2L, CCND1, MLEC, RARS, SLC7A5, MANF, RAE1, CHD4, BYSL, GTF3C2, TRAP1, KIAA0114, CDC25B, SF3B2, SMARCA4, DDX11, LMNB2, DHCR24, SAFB, CTPS, DUSP1, DDX10, CDC123, NDRG1, EIF2B2, FAM38A, SEC24C, RABEPK, FGFR3, SSRP1, and SLC1A3;

(b) normalizing the expression data for each of the markers to a control, and

(c) selecting the subject as a PARP inhibitor/DNA damaging chemotherapeutic-sensitive subject when expression of the combination of markers is increased compared to a reference value, and

selecting the subject as a PARP inhibitor/DNA damaging chemotherapeutic-insensitive subject when expression of the combination of markers is not changed or decreased compared to the reference value.

17. The method of paragraph 16, further comprising assaying the biological sample for at least one additional marker selected from the group consisting of: GCSH, BOP1, XRCC5, CCDC85B, H1FX, FDFT1, DNAJC7, PRKACA, MAP2K5, SNRPA1, RELA, RXRA, KDM5C, PWP2, NME3, FARSA, DNTTIP2, MRPS12, SCRIB, PPAT, CDC37, SLC29A1, SFRS11, SMAD6, EIF4H, VDAC1, NMT1, POLRMT, POLD1, CHAF1A, TAF1C, TCOF1, ALG3, CAD, RASSF7, MTHFD1, CCNE1, HSF1, PSMD1, AGPAT2, PHKG2, PKN1, GAL, SCAP, EIF4G1, DDT, PMPCA, RRP1B, FKBP4, NUP214, ATIC, FASN, AP3D1, RPIA, NSDHL, BRD2, PDCD11, SON, HSP90B1, and ANXA11.
18. The method of paragraph 16 or 17, wherein the biological sample is assayed for expression of all of the markers SFRS11, EZR, PKN1, SOX4, EMG1, RELA, AIMP2, NARS, HSP90B1, SNRP70, NME3, CLTA, CAD, EIF3B, EIF3E, UBAP2L, SF3A3, HK1, TCOF1, CKAP4, CCNE1, NOLC1, HCFC1, PPM1F, BMS1L, RASSF7, FDFT1, FARSLA, POLG, MCM2, VDAC1, U2AF1, MOGS, ATIC, CHAF1A, SKIV2L, GCSH, GAL, CCDC85B, CCND1, MLEC, RARS, WBSCR1, FKBP4, SLC7A5, MTHFD1, NSDHL, PWP2H, FASN, MANF, EIF4G1, JARID1C, SCRIB, SCAP, PSMD1, RAE1, CHD4, SLC29A1, BYSL, GTF3C2, TRAP1, PRKACA, KIAA0179, KIAA0114, POLD1, DNTTIP2, BOP1, CDC25B, MRPS12, SF3B2, NUP214, PHKG2, AGPAT2, SMARCA4, PPAT, SNRPA1, DDX11, RPIA, ALG3, LMNB2, TAF1C, DDT, DHCR24, SAFB, CDC37, H1FX, AP3D1, DNAJC7, HSF1, CTPS, DUSP1, DDX10, PDCD11, CDC123, RXRA, NDRG1, EIF2B2, ANXA11, FAM38A, XRCC5, SEC24C, PMPCA, POLRMT, RABEPK, SMAD6, NMT1, SON, MAP2K5, SLC1A3, FGFR3, SSRP1, and BRD2.
19. The method of paragraph 16, 17 or 18, wherein the control is the expression level of the total set of markers assayed.
20. The method of any one of paragraphs 16-19, further comprising a step of calculating a metagene value to represent the normalized expression of the combination of markers.
21. The method of any one of paragraphs 16-20, wherein the step of calculating the metagene comprises linear combination of the expression values of each marker in the combination of markers.
22. The method of any one of paragraphs 16-21, wherein the linear combination has a coefficient of 1 for each marker.
23. The method of any one of paragraphs 16-22, wherein the biological sample comprises a tumor sample.
24. The method of any one of paragraphs 16-23, wherein the subject carries a mutation in the BRCA1 gene.
25. The method of any one of paragraphs 16-24, wherein the subject carries wild-type BRCA1 gene.
26. The method of any one of paragraphs 16-25, wherein the BRCA1 status of the subject is unknown.
27. The method of any one of paragraphs 16-26, wherein the cancer is breast cancer.
28. The method of any one of paragraphs 16-27, wherein the expression assayed is mRNA expression.
29. The method of any one of paragraphs 16-28, wherein the expression assayed is protein expression.
30. A method of determining reduced BRCA1 pathway function in a cancer cell, the method comprising:

(a) assaying a biological sample comprising a cancer cell for expression of at least the following markers: EZR, SOX4, EMG1, NARS, SNRNP70, CLTA, EIF3B, EIF3E, UBAP2L, SF3A3, HK1, CKAP4, NOLC1, HCFC1, PPM1F, BMS1, AIMP2, POLG, MCM2, U2AF1, MOGS, SKIV2L, CCND1, MLEC, RARS, SLC7A5, MANF, RAE1, CHD4, BYSL, GTF3C2, TRAP1, KIAA0114, CDC25B, SF3B2, SMARCA4, DDX11, LMNB2, DHCR24, SAFB, CTPS, DUSP1, DDX10, CDC123, NDRG1, EIF2B2, FAM38A, SEC24C, RABEPK, FGFR3, SSRP1, and SLC1A3, and

(b) normalizing the expression data for each of the markers to a control,

wherein the cancer cell is determined to have reduced BRCA1 pathway function when the metagene value is increased compared to a reference value, and wherein the cancer cell is determined not to have reduced BRCA1 pathway function when the metagene value is not changed or reduced compared to a reference value.

31. The method of paragraph 30, wherein the control is the expression level of the total set of markers assayed.
32. The method of paragraph 30 or 31, wherein the control is the expression level of at least one of the following markers: ACTB, HBMS, RPLPO, UBC, ZFAND3, ZNF212, FBXO34, DDX3X, SDHAF1, EIF4G2, USP12, MUS81, HDAC6, CTDSP2, COPS7A, CTCF, ANAPC5, VPS11, DPP8, UBAP1, HBXIP, UBA1, PRR13, TRA2B, OPA1, RAB7A, MAPRE1, RXRB, SNW, TTC31, PUM2, C12orf41, SAP130, and BFAR.
33. The method of paragraph 30, 31, or 32, further comprising a step of calculating a metagene value to represent the normalized expression of the combination of markers.
34. The method of any one of paragraphs 30-33, wherein the step of calculating the metagene comprises linear combination of the expression values of each marker in the combination of markers.
35. The method of any one of paragraphs 30-34, wherein the linear combination has a coefficient of 1 for each marker.
36. The method of any one of paragraphs 30-35, wherein the biological sample comprises a tumor sample.
37. The method of any one of paragraphs 30-36, further comprising assaying the biological sample for at least one additional marker selected from the group consisting of: GCSH, BOP1, XRCC5, CCDC85B, H1FX, FDFT1, DNAJC7, PRKACA, MAP2K5, SNRPA1, RELA, RXRA, KDM5C, PWP2, NME3, FARSA, DNTTIP2, MRPS12, SCRIB, PPAT, CDC37, SLC29A1, SFRS11, SMAD6, EIF4H, VDAC1, NMT1, POLRMT, POLD1, CHAF1A, TAF1C, TCOF1, ALG3, CAD, RASSF7, MTHFD1, CCNE1, HSF1, PSMD1, AGPAT2, PHKG2, PKN1, GAL, SCAP, EIF4G1, DDT, PMPCA, RRP1B, FKBP4, NUP214, ATIC, FASN, AP3D1, RPIA, NSDHL, BRD2, PDCD11, SON, HSP90B1, and ANXA11.
38. The method of any one of paragraphs 30-37, wherein the biological sample is assayed for expression of all of the markers SFRS11, EZR, PKN1, SOX4, EMG1, RELA, AIMP2, NARS, HSP90B1, SNRP70, NME3, CLTA, CAD, EIF3B, EIF3E, UBAP2L, SF3A3, HK1, TCOF1, CKAP4, CCNE1, NOLC1, HCFC1, PPM1F, BMS1L, RASSF7, FDFT1, FARSLA, POLG, MCM2, VDAC1, U2AF1, MOGS, ATIC, CHAF1A, SKIV2L, GCSH, GAL, CCDC85B, CCND1, MLEC, RARS, WBSCR1, FKBP4, SLC7A5, MTHFD1, NSDHL, PWP2H, FASN, MANF, EIF4G1, JARID1C, SCRIB, SCAP, PSMD1, RAE1, CHD4, SLC29A1, BYSL, GTF3C2, TRAP1, PRKACA, KIAA0179, KIAA0114, POLD1, DNTTIP2, BOP1, CDC25B, MRPS12, SF3B2, NUP214, PHKG2, AGPAT2, SMARCA4, PPAT, SNRPA1, DDX11, RPIA, ALG3, LMNB2, TAF1C, DDT, DHCR24, SAFB, CDC37, H1FX, AP3D1, DNAJC7, HSF1, CTPS, DUSP1, DDX10, PDCD11, CDC123, RXRA, NDRG1, EIF2B2, ANXA11, FAM38A, XRCC5, SEC24C, PMPCA, POLRMT, RABEPK, SMAD6, NMT1, SON, MAP2K5, SLC1A3, FGFR3, SSRP1, and BRD2.
39. The method of any one of paragraphs 30-38, wherein the subject is known to carry a mutation in the BRCA1 gene.
40. The method of any one of paragraphs 30-39, wherein the subject is known to carry a wild-type BRCA1 gene.
41. The method of any one of paragraphs 30-40, wherein the BRCA1 status of the subject is unknown.
42. The method of any one of paragraphs 30-41, wherein the cancer is breast cancer.
43. The method of any one of paragraphs 30-42, wherein the expression assayed is mRNA expression.
44. The method of any one of paragraphs 30-43, wherein the expression assayed is protein expression.
45. The method of any one of the preceding paragraphs, wherein the PARP inhibitor is olaparib.
46. The method of any one of the preceding paragraphs, wherein the DNA damage inducing chemotherapeutic agent is temozolomide.
47. A composition comprising an array consisting essentially of probes directed to the following genes: EZR, SOX4, EMG1, NARS, SNRNP70, CLTA, EIF3B, EIF3E, UBAP2L, SF3A3, HK1, CKAP4, NOLC1, HCFC1, PPM1F, BMS1, AIMP2, POLG, MCM2, U2AF1, MOGS, SKIV2L, CCND1, MLEC, RARS, SLC7A5, MANF, RAE1, CHD4, BYSL, GTF3C2, TRAP1, KIAA0114, CDC25B, SF3B2, SMARCA4, DDX11, LMNB2, DHCR24, SAFB, CTPS, DUSP1, DDX10, CDC123, NDRG1, EIF2B2, FAM38A, SEC24C, RABEPK, FGFR3, SSRP1, and SLC1A3; and attached to a solid support.
48. The composition of paragraph 47, wherein the probes are cDNA probes.
49. A composition comprising an array consisting essentially of probes directed to the following genes: SFRS11, EZR, PKN1, SOX4, EMG1, RELA, AIMP2, NARS, HSP90B1, SNRP70, NME3, CLTA, CAD, EIF3B, EIF3E, UBAP2L, SF3A3, HK1, TCOF1, CKAP4, CCNE1, NOLC1, HCFC1, PPM1F, BMS1L, RASSF7, FDFT1, FARSLA, POLG, MCM2, VDAC1, U2AF1, MOGS, ATIC, CHAF1A, SKIV2L, GCSH, GAL, CCDC85B, CCND1, MLEC, RARS, WBSCR1, FKBP4, SLC7A5, MTHFD1, NSDHL, PWP2H, FASN, MANF, EIF4G1, JARID1C, SCRIB, SCAP, PSMD1, RAE1, CHD4, SLC29A1, BYSL, GTF3C2, TRAP1, PRKACA, KIAA0179, KIAA0114, POLD1, DNTTIP2, BOP1, CDC25B, MRPS12, SF3B2, NUP214, PHKG2, AGPAT2, SMARCA4, PPAT, SNRPA1, DDX11, RPIA, ALG3, LMNB2, TAF1C, DDT, DHCR24, SAFB, CDC37, H1FX, AP3D1, DNAJC7, HSF1, CTPS, DUSP1, DDX10, PDCD11, CDC123, RXRA, NDRG1, EIF2B2, ANXA11, FAM38A, XRCC5, SEC24C, PMPCA, POLRMT, RABEPK, SMAD6, NMT1, SON, MAP2K5, SLC1A3, FGFR3, SSRP1, and BRD2.
50. The composition of paragraph 49, wherein the probes are cDNA probes.
51. A method of treating cancer in a subject, the method comprising: administering to a subject a therapeutically effective amount of a poly ADP ribose polymerase (PARP) inhibitor and a DNA damage-inducing chemotherapeutic agent when the subject has been determined to have increased expression of a metagene comprising a combination of markers selected from the group consisting of EZR, SOX4, EMG1, NARS, SNRNP70, CLTA, EIF3B, EIF3E, UBAP2L, SF3A3, HK1, CKAP4, NOLC1, HCFC1, PPM1F, BMS1, AIMP2, POLG, MCM2, U2AF1, MOGS, SKIV2L, CCND1, MLEC, RARS, SLC7A5, MANF, RAE1, CHD4, BYSL, GTF3C2, TRAP1, KIAA0114, CDC25B, SF3B2, SMARCA4, DDX11, LMNB2, DHCR24, SAFB, CTPS, DUSP1, DDX10, CDC123, NDRG1, EIF2B2, FAM38A, SEC24C, RABEPK, FGFR3, SSRP1, and SLC1A3 as compared to a reference value, and/or

not administering a PARP inhibitor and a DNA damage-inducing chemotherapeutic agent when the subject has been determined to have unchanged or decreased expression of a metagene comprising a combination of markers selected from the group consisting of EZR, SOX4, EMG1, NARS, SNRNP70, CLTA, EIF3B, EIF3E, UBAP2L, SF3A3, HK1, CKAP4, NOLC1, HCFC1, PPM1F, BMS1, AIMP2, POLG, MCM2, U2AF1, MOGS, SKIV2L, CCND1, MLEC, RARS, SLC7A5, MANF, RAE1, CHD4, BYSL, GTF3C2, TRAP1, KIAA0114, CDC25B, SF3B2, SMARCA4, DDX11, LMNB2, DHCR24, SAFB, CTPS, DUSP1, DDX10, CDC123, NDRG1, EIF2B2, FAM38A, SEC24C, RABEPK, FGFR3, SSRP1, and SLC1A3 as compared to a reference value.

52. The method of paragraph 51, further comprising assaying a biological sample from the subject for the expression of the metagene.
53. The method of paragraph 51, 52, or 53, further comprising obtaining a biological sample comprising cancer cells from the subject.

54. A method of treating cancer in a human in need thereof, the method comprising
(a) selecting a human who has increased expression compared to a reference value of the combination of at least five markers selected from the following markers: EZR, SOX4, EMG1, NARS, SNRNP70, CLTA, EIF3B, EIF3E, UBAP2L, SF3A3, HK1, CKAP4, NOLC1, HCFC1, PPM1F, BMS1, AIMP2, POLG, MCM2, U2AF1, MOGS, SKIV2L, CCND1, MLEC, RARS, SLC7A5, MANF, RAE1, CHD4, BYSL, GTF3C2, TRAP1, KIAA0114, CDC25B, SF3B2, SMARCA4, DDX11, LMNB2, DHCR24, SAFB, CTPS, DUSP1, DDX10, CDC123, NDRG1, EIF2B2, FAM38A, SEC24C, RABEPK, FGFR3, SSRP1, and SLC1A3 in a biological sample; and

(b) administering to the human a therapeutically effective amount of a poly ADP ribose polymerase (PARP) inhibitor and a DNA damage-inducing chemotherapeutic agent.

55. The method of paragraph 54, wherein the step of selecting comprises assaying a biological sample from the human for a nucleotide sequence encoding the at least five markers.
56. The method of paragraph 54 or 55, wherein the assaying comprises nucleic acid amplification and optionally one or more methods selected from sequencing, next generation sequencing, nucleic acid hybridization, and allele-specific amplification.
57. The method of paragraph 54, 55, or 56, wherein the assaying is performed in a multiplex format.
58. The method of any one of paragraphs 54-57, further comprising obtaining the biological sample from the human.
59. The method of any one of paragraphs 54-58, wherein the biological sample comprises serum, blood, feces, tissue, a cell, urine and/or saliva of the human.
60. A method of treating cancer in a human in need thereof, the method comprising

(a) selecting a human who has comparable expression compared to a reference value of the combination of at least five markers selected from the following markers: EZR, SOX4, EMG1, NARS, SNRNP70, CLTA, EIF3B, EIF3E, UBAP2L, SF3A3, HK1, CKAP4, NOLC1, HCFC1, PPM1F, BMS1, AIMP2, POLG, MCM2, U2AF1, MOGS, SKIV2L, CCND1, MLEC, RARS, SLC7A5, MANF, RAE1, CHD4, BYSL, GTF3C2, TRAP1, KIAA0114, CDC25B, SF3B2, SMARCA4, DDX11, LMNB2, DHCR24, SAFB, CTPS, DUSP1, DDX10, CDC123, NDRG1, EIF2B2, FAM38A, SEC24C, RABEPK, FGFR3, SSRP1, and SLC1A3 in a biological sample; and

(b) administering to the human a therapeutically effective amount of a cancer treatment that does not comprise poly ADP ribose polymerase (PARP) inhibitor and a DNA damage-inducing chemotherapeutic agent.

61. The method of paragraph 60, wherein the step of selecting comprises assaying a biological sample from the human for a nucleotide sequence encoding the at least five markers.
62. The method of paragraph 60 or 61, wherein the assaying comprises nucleic acid amplification and optionally one or more methods selected from sequencing, next generation sequencing, nucleic acid hybridization, and allele-specific amplification.
63. The method of paragraph 60, 61, or 62, wherein the assaying is performed in a multiplex format.
64. The method of any one of paragraphs 60-63, further comprising obtaining the biological sample from the human.
65. The method of any one of paragraphs 60-64, wherein the biological sample comprises serum, blood, feces, tissue, a cell, urine and/or saliva of the human.

### EXAMPLES

### Example 1

### Materials and Methods

### Patients and Clinical Samples

Patient and primary tumor characteristics are presented in Table 5.

| Brain Mets | AGE | ER | Her2 | MGH Primary Cases | AGE | ER | Her2 |
|---|---|---|---|---|---|---|---|
| J3 | 51 | − | 3+ | MGH16 | 51 | − | 3+ |
| BT25 | 52.8 | + | 3+ | MGH42 | 50 | + | 3+ |
| J12 | 55.4 | − | 3+ | MGH46 | 56 | − | 3+ |
| BT16 | 43.7 | − | 3+ | MGH133 | 44 | − | 3+ |
| J8 | 42.6 | + | 3+ | MGH153 | 46 | + | 3+ |
| BT34 | 55.5 | + | 3+ | MGH351 | 54 | + | 3+ |
| J1 | 37.6 | − | 3+ | MGH1104 | 32 | − | 3+ |
| BT11 | 71.7 | − | 3+ | MGH574 | 73 | − | 3+ |
| J2 | 51.5 | − | 3+ | MGH434 | 51 | − | 3+ |
| BT30 | 58.8 | − | 3+ | MGH450-3 | 60 | − | 3+ |
| J4 | 43.2 | − | 3+ | MGH421 | 71 | − | 3+ |
| BT57 | 66 | − | 3+ | MGH482 | 66 | − | 3+ |
| J5 | 40.7 | | 3+ | MGH963 | 41 | | 3+ |
| BT51 | 38.5 | − | 3+ | MGH455 | 61 | − | 3+ |
| BT31 | 57.2 | − | 3+ | MGH1084 | 57 | − | 3+ |
| J7 | 49.1 | − | 3+ | MGH1038 | 50 | − | 3+ |
| J10 | 43.6 | − | 3+ | MGH1057 | 47 | − | 3+ |
| J11 | 49.1 | − | 3+ | MGH674 | 53 | − | 3+ |
| BT40 | 50.4 | + | 3+ | MGH1102 | 51 | + | 3+ |

The HER2 status was assessed by HER2 immunohistochemistry (IHC) and/or gene amplification, and tumor grading were determined as described previouslyl¹⁰. These primary, nonmetastatic and brain metastatic specimens were matched based upon the patient age upon primary tumor detection and the estrogen receptor status of the primary tumor. The nonmetastatic HER2+ primary breast cancer specimens were obtained from patients with no overall relapse and consisted of fresh-frozen biopsies obtained from the Massachusetts General Hospital in 2006. Brain metastasis was an early first-site of relapse for 19 HER2+ breast cancer patients, and these specimens consisted of fresh-frozen biopsies obtained from the M.D. Anderson Cancer Center between 1998 and 2001; the matched primary tumor specimen for these patients was not available. The study was approved by the human research committees of M.D. Anderson Cancer Center and the Massachusetts General Hospital in accordance with the National Institutes of Health human research study guidelines.

Laser Capture Microdissection, RNA Extraction, and Microarray Hybridization

RNA was isolated from a highly enriched population of 4,000-5,000 malignant epithelial cells procured by laser capture microdissection and was hybridized to Affymetrix X3P GeneChips as previously described“. The data was deposited in the NCBI Gene Expression Omnibus” and are accessible through GEO Series accession number GSE43837.

### Gene Set Enrichment Analysis

Computation of gene expression was done using the MASS algorithm as implemented in the call.expers function in version 2.14.05 of the simple affy package of BIOCONDUCTOR™. GSEA analysis was performed using version 2.0 of GSEA run on all the gene sets in version 2.5 of MSigDB (BROAD INSTITUTE™).

### Calculation of BRCA1 Deficient-Like Metagene Value

All the genes in the BRCA1_OVEREXP_DN gene set, which was experimentally derived as described¹⁵, in Version 2.5 of the MSigDB (BROAD INSTITUTE™) were mapped as described above to microarray identifiers. The gene expression values for all those identifiers were then averaged to form the BRCA1 Deficient-Like metagene. Specific probes measured are indicated in Table 1 for each figure.

### Mapping Gene Symbols to Microarray Identifiers

Gene symbols were mapped to Entrez GenelDs using the 02/02/2008 version of the gene info file from ftp.ncbi.nlm.nih gov/gene/DATA. First the “Symbol” column was searched and, if that failed, the “Synonyms” column was searched. To map an Entrez GeneID to AFFYMETRIX™ HG-U133A probe set identifiers, version na24 of the annotation file from the AFFYMETRIX™ web site was used. The “Entrez Gene” column of that annotation file was augmented by trying to fill empty entries by using the corresponding entries in the “UniGene ID” and “Representative Public ID” columns to search the file Hs.data from build 209 of Unigene and the 02/02/2008 version of the gene2accession file from ftp.ncbi.nlm.nih.gov/gene/DATA. An Entrez GeneID was then mapped to every probe set identifier that had the Entrez GeneID in the augmented “Entrez Gene” column. To map Entrez GenelDs to Rosetta spot IDs, the inventors used www.rii.com/publications/data/ArrayNomenclature_contig_accession.xls (downloaded Feb. 2, 2008), the file Hs.data from build 209 of Unigene and the 02/02/2008 version of the gene2accession file from ftp.ncbi.nlm.nih.gov/gene/DATA.

### Sequencing of Genomic BRCA1

All 22 coding exons of the BRCA1 (NM_007294.3) gene were amplified and sequenced in 33 fragments in tumor DNA. Primers were designed using PRIMER 3™ software¹³ to cover at least 20 base pairs at each 5′ and 3′ side of the exons. The amplified DNA fragments were sequenced by using the BIGDYE TERMINAROT CYCLE SEQUENCING™ kit on an ABI 3500×1 DNA ANALYZER™ (APPLIED BIOSYSTEMS™, Foster City, Calif., USA). Sequencing chromatograms generated by the analyzer were examined for variant detection using MUTATION SURVEYOR software (SOFTGENETICS LLC™, PA, USA).

### Statistical Methods for Correlative Analyses

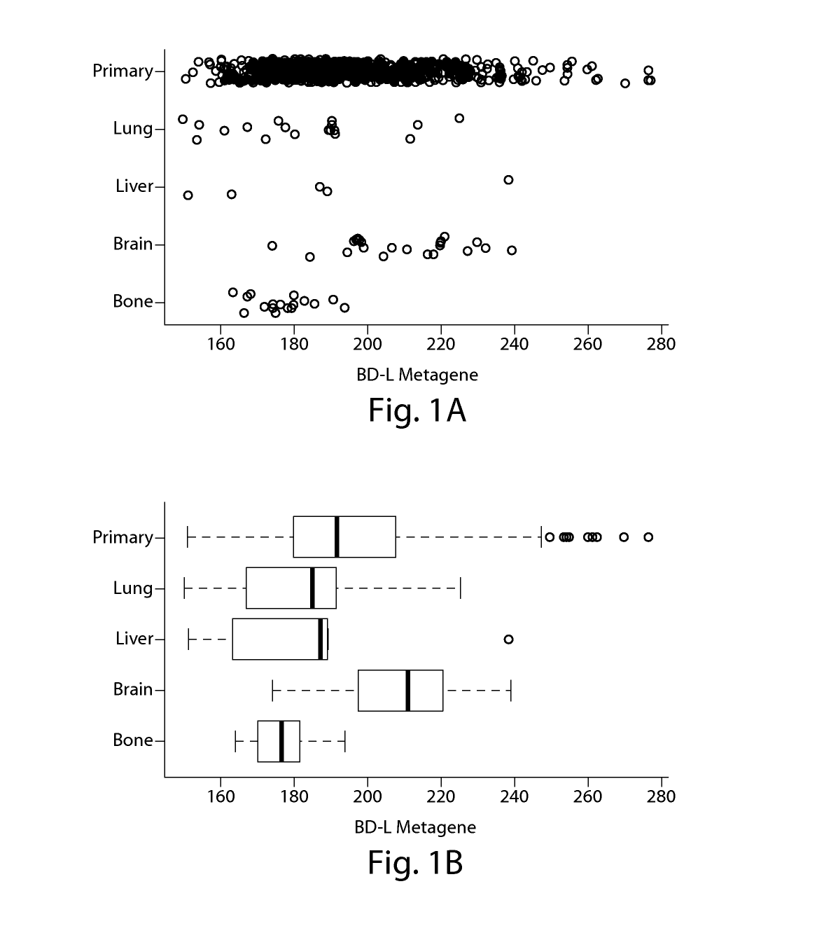

The p-values quoted for FIGS. 1, 2, and 3A were obtained by applying the Wilcoxon test to all pairs within the figure and correcting the resulting p-values for multiple hypothesis testing using the Holm method¹⁴.

### Cell Culture and Pharmacologic Inhibition Assay

All cell lines were obtained and maintained as previously described¹⁵. Independent pharmacologic inhibition assays were conducted in triplicate for each cell line. Cells were seeded at 20,000 per well in a 24-well plate. After 24 hours, cells were treated in triplicate with indicated concentrations of: DMSO, temozolomide (SIGMA ALDRICH™, T2577), or AZD-2281 (SELLECK CHEMICALS™, S1060). After five days of incubation, cells were fixed in 4% formaldehyde and stained with 1% Crystal Violet (SIGMA ALDRICH™, C0775) for ten minutes at room temperature. Cells were then washed to remove unincorporated dye and plates were inverted to dry overnight. Incorporated dye was extracted with 10% acetic acid and OD595 measurements were obtained within a linear range. Treated cells were normalized to the vehicle-treated control to obtain mean percent viability.

### Example 2

### Results

To identify gene expression patterns that differentiate HER2+ breast cancer brain metastases from HER2+ nonmetastatic primary tumors, the inventors performed a comparative gene expression analysis between 19 brain metastasis specimens from breast cancer patients with the brain as a first-site metastasis and 19 non-metastatic primary breast tumor specimens. These specimens were matched based upon the age of patient at initial detection and the HER2 and estrogen receptor (ER) status of the primary tumors (Table 1). Although the primary tumors matched to the brain metastatic specimens were not available for gene expression or sequencing analysis, the direct comparison of brain metastases to nonmetastatic primary tumors gives insight into the key molecular pathways underlying outgrowth in the brain microenvironment. Additionally, identification of such pathways may be masked by heterogeneity in the primary tumor or can be later acquired following the initial metastatic seeding by adaptation or mutation.

To compare gene expression, RNA derived from microdissected tissue was hybridized to AFFYMETRIX™ X3P GeneChips and the resulting data was subjected to bioinformatic analyses. Standard MASS pre-processing of the data with a t-test comparison and a false discovery rate set at 0.25 failed to identify individually differentially expressed genes between the brain metastatic specimens and the non-patient matched primary breast cancer specimens.

As no significant differential expression for individual genes was discovered, a Gene Set Enrichment Analysis (GSEA) using Version 2.5 the BROAD INSTITUTE™ MSigDB (Molecular Signatures Database) was conducted to determine if there were modulations of gene sets that comprise annotated biochemical pathways¹⁶. The analysis yielded 22 enriched gene sets with a false discovery rate of q-value <0.25 (Table 6).

TABLE 6
22 enriched gene sets with a false discovery rate of q-value <0.25.

| | | | | | | | RANK | LEAD- |
|---|---|---|---|---|---|---|---|---|
| | | | | NOM | FDR | FWER | AT | ING |
| NAME | SIZE | ES | NES | p-val | q-val | p-val | MAX | EDGE |
| BRCA1_OVEREXP_ | 110 | −0.56325257 | −2.0555308 | 0 | 0.08357126 | 0.066 | 3016 | tags = |
| DN | | | | | | | | 47%, |
| | | | | | | | | list = 1 |
| | | | | | | | | 5%, |
| | | | | | | | | signal = |
| | | | | | | | | 55% |
| CANTHARIDIN_DN | 50 | −0.57416445 | −1.9845413 | 0 | 0.12829918 | 0.156 | 3456 | tags = |
| | | | | | | | | 46%, |
| | | | | | | | | list = 1 |
| | | | | | | | | 7%, |
| | | | | | | | | signal = |
| | | | | | | | | 55% |
| ZHAN_MULTIPLE_ | 62 | −0.5245591 | −1.9430114 | 0 | 0.15066467 | 0.238 | 3331 | tags = |
| MYELOMA_VS_ | | | | | | | | 47%, |
| NORMAL_UP | | | | | | | | list = 1 |
| | | | | | | | | 6%, |
| | | | | | | | | signal = |
| | | | | | | | | 56% |
| HDACI_COLON_ | 37 | −0.53656936 | −1.8751472 | 0.0078125 | 0.15154383 | 0.439 | 1762 | tags = |
| CUR24HRS_UP | | | | | | | | 32%, |
| | | | | | | | | list = |
| | | | | | | | | 9%, |
| | | | | | | | | signal = |
| | | | | | | | | 35% |
| MOOTHA_VOXPHOS | 77 | −0.55048025 | −1.8133028 | 0.00782779 | 0.15416802 | 0.638 | 4598 | tags = |
| | | | | | | | | 53%, |
| | | | | | | | | list = 2 |
| | | | | | | | | 2%, |
| | | | | | | | | signal = |
| | | | | | | | | 68% |
| UVC_XPCS_8HR_UP | 57 | −0.4898372 | −1.8840362 | 0 | 0.15997946 | 0.411 | 2948 | tags = |
| | | | | | | | | 44%, |
| | | | | | | | | list = 1 |
| | | | | | | | | 4%, |
| | | | | | | | | signal = |
| | | | | | | | | 51% |
| POMEROY_MD_ | 24 | −0.5974129 | −1.8143351 | 0.00390625 | 0.16399892 | 0.63 | 3945 | tags = |
| TREATMENT_ | | | | | | | | 46%, |
| GOOD_VS_ | | | | | | | | list = 1 |
| POOR_DN | | | | | | | | 9%, |
| | | | | | | | | signal = |
| | | | | | | | | 57% |
| HSA05050_ | 15 | −0.623107 | −1.7948993 | 0.00583658 | 0.1640294 | 0.686 | 1918 | tags = |
| DENTAT- | | | | | | | | 40%, |
| ORUBRO- | | | | | | | | list = |
| PALLIDOLUYSIAN_ | | | | | | | | 9%, |
| ATROPHY | | | | | | | | signal = |
| | | | | | | | | 44% |
| UVC_XPCS_ALL_UP | 59 | −0.4958749 | −1.8953222 | 0 | 0.16551158 | 0.372 | 2948 | tags = |
| | | | | | | | | 44%, |
| | | | | | | | | list = 1 |
| | | | | | | | | 4%, |
| | | | | | | | | signal = |
| | | | | | | | | 51% |
| HSA05110_CHOLERA_ | 40 | −0.5189111 | −1.7981354 | 0.003861 | 0.17060181 | 0.68 | 3893 | tags = |
| INFECTION | | | | | | | | 50%, |
| | | | | | | | | list = 1 |
| | | | | | | | | 9%, |
| | | | | | | | | signal = |
| | | | | | | | | 61% |
| ELECTRON_ | 98 | −0.51865566 | −1.8155646 | 0.01335878 | 0.17477453 | 0.623 | 3892 | tags = |
| TRANSPORT_ | | | | | | | | 44%, |
| CHAIN | | | | | | | | list = 1 |
| | | | | | | | | 9%, |
| | | | | | | | | signal = |
| | | | | | | | | 54% |
| AMINOACYL_TRNA_ | 23 | −0.6677216 | −1.8204464 | 0.01629328 | 0.18069182 | 0.609 | 2187 | tags = |
| BIOSYNTHESIS | | | | | | | | 48%, |
| | | | | | | | | list = 1 |
| | | | | | | | | 1%, |
| | | | | | | | | signal = |
| | | | | | | | | 53% |
| HSA00190_ | 113 | −0.5231598 | −1.827699 | 0.006 | 0.18265781 | 0.591 | 3611 | tags = |
| OXIDATIVE_ | | | | | | | | 44%, |
| PHOSPHORYLATION | | | | | | | | list = 1 |
| | | | | | | | | 7%, |
| | | | | | | | | signal = |
| | | | | | | | | 53% |
| HDACI_COLON_ | 62 | −0.49318787 | −1.8325484 | 0.00398406 | 0.19129947 | 0.579 | 4121 | tags = |
| CUR48HRS_UP | | | | | | | | 48%, |
| | | | | | | | | list = 2 |
| | | | | | | | | 0%, |
| | | | | | | | | signal = |
| | | | | | | | | 60% |
| OXIDATIVE_ | 58 | −0.51359004 | −1.7707858 | 0.01757813 | 0.19722769 | 0.752 | 2973 | tags = |
| PHOSPHORYLATION | | | | | | | | 40%, |
| | | | | | | | | list = 1 |
| | | | | | | | | 4%, |
| | | | | | | | | signal = |
| | | | | | | | | 46% |
| MENSSEN_MYC_UP | 31 | −0.7032262 | −1.8978591 | 0.00390625 | 0.20059656 | 0.36 | 1670 | tags = |
| | | | | | | | | 52%, |
| | | | | | | | | list = |
| | | | | | | | | 8%, |
| | | | | | | | | signal = |
| | | | | | | | | 56% |
| FLECHNER_ | 153 | −0.45394486 | −1.8381206 | 0 | 0.20321022 | 0.565 | 4160 | tags = |
| KIDNEY_ | | | | | | | | 44%, |
| TRANSPLANT_ | | | | | | | | list = 2 |
| WELL_PBL_UP | | | | | | | | 0%, |
| | | | | | | | | signal = |
| | | | | | | | | 55% |
| CMV_ | 46 | −0.43538764 | −1.7482822 | 0.00199601 | 0.23084806 | 0.807 | 3727 | tags = |
| HCMV_ | | | | | | | | 50%, |
| TIMECOURSE_ | | | | | | | | list = 1 |
| 14HRS_UP | | | | | | | | 8%, |
| | | | | | | | | signa l= |
| | | | | | | | | 61% |
| TRNA_SYNTHETASES | 19 | −0.6983192 | −1.7365746 | 0.01810865 | 0.23295872 | 0.821 | 2187 | tags = |
| | | | | | | | | 53%, |
| | | | | | | | | list = 1 |
| | | | | | | | | 1%, |
| | | | | | | | | signal = |
| | | | | | | | | 59% |
| HDACI_COLON_ | 63 | −0.51326543 | −1.7380248 | 0.00984252 | 0.24220917 | 0.819 | 3892 | tags = |
| SUL24HRS_UP | | | | | | | | 44%, |
| | | | | | | | | list = 1 |
| | | | | | | | | 9%, |
| | | | | | | | | signal = |
| | | | | | | | | 55% |
| ET743_RESIST_UP | 17 | −0.60277396 | −1.7163161 | 0.0113852 | 0.24813692 | 0.866 | 2587 | tags = |
| | | | | | | | | 41%, |
| | | | | | | | | list = 1 |
| | | | | | | | | 3%, |
| | | | | | | | | signal = |
| | | | | | | | | 47% |
| CREB_BRAIN_ | 24 | −0.61414903 | −1.7108434 | 0.00194175 | 0.24931273 | 0.875 | 4038 | tags = |
| 2WKS_UP | | | | | | | | 50%, |
| | | | | | | | | list = 2 |
| | | | | | | | | 0%, |
| | | | | | | | | signal = |
| | | | | | | | | 62% |

The top gene set identified was BRCA1_OVEREXP_DN, which is comprised of probe sets that were downregulated by 2-4 fold after inducible expression of BRCA1 in the BRCA1-low, ER+ EcR-293 human embryonal kidney epithelial cell line¹⁷. A significant correlation of the HER2+ breast cancer brain metastases with a BRCA1-related signature was unexpected as several studies have reported a low frequency of HER2 expression in tumors of BRCA1 mutation carriers¹⁸⁻²°. Furthermore, sequencing analysis for 17 of the 19 HER2+ brain metastatic specimen for which there was suitable remaining material identified no previously known pathogenic or novel potentially pathogenic variants (Frame shift insertion/deletion, nonsense or essential splicing site variants) as classified by IARC recommendations²¹. Because this signature was identified as enriched in brain metastases from HER2+ breast cancer patients without pathogenic variants, the inventors designated the BRCA1_OVEREXP_DN signature as the “BRCA1 Deficient-Like” (BD-L) metagene.

To further explore the nature of this signature, the inventors hypothesized that a positive correlation with the BD-L signature can identify sporadic breast cancer patients with clinical outcomes similar to BRCA1-mutation carriers, and that a positive correlation indicates an underlying deficiency in a BRCA1 functional or regulated pathway, either directly through BRCA1 or indirectly through a cooperating factor.

To validate the inventors' original observation that the BRCA1 Deficient-Like metagene is enriched in breast cancer-derived brain metastases, gene expression data from an independent cohort consisting of 615 primary breast cancer specimens as well as breast cancer metastasis specimens from brain (n=19), lung (n=18), liver (n=5), and bone (n=5) was assessed for correlation with BD-L²². As demonstrated in FIG. 1, a higher mean BD-L metagene value was observed in metastases to the brain as compared to primary tumors (p-value=0.0043), bone metastases (p-value=4×10⁻⁶), and lung metastases (p-value=0.001), but not when compared to liver (p-value=0.38).

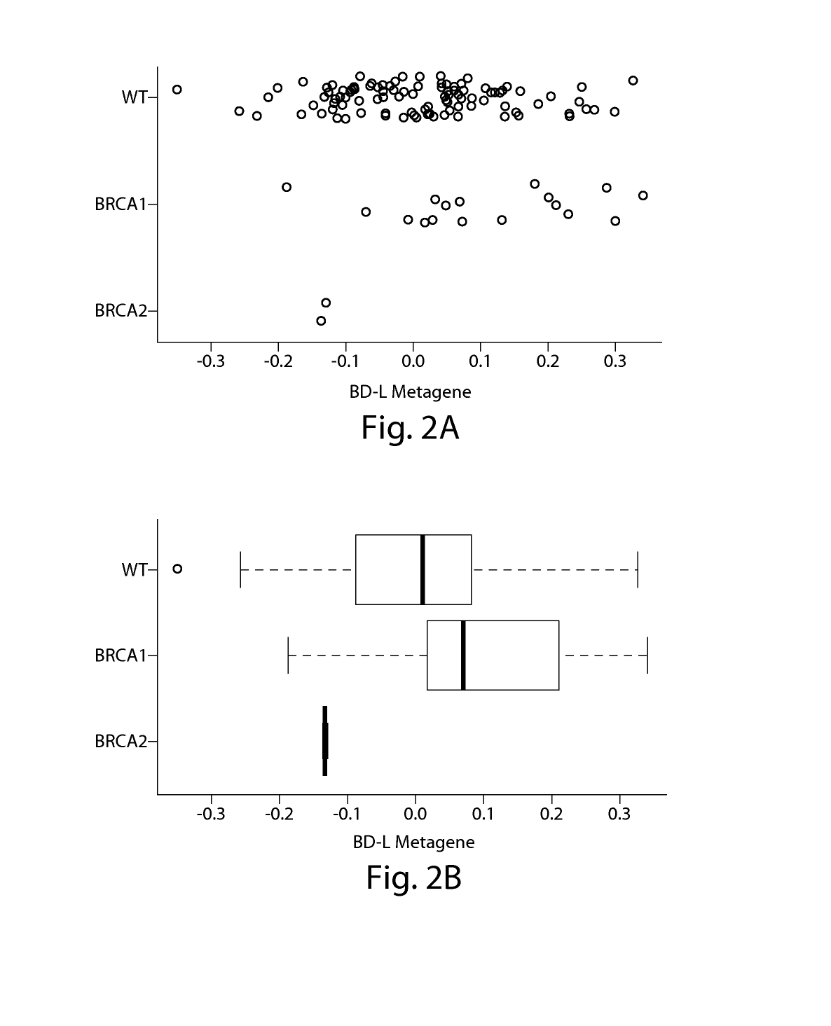

Having confirmed an enrichment of BD-L metagene value in brain metastases compared to primary tumors, the inventors then hypothesized that a metagene of BRCA1 deficiency would also demonstrate increased values in primary tumor specimen derived from mutant BRCA1 carriers compared to non-carriers. When a publicly available gene expression dataset was interrogated²³, a significantly higher mean BD-L value was found in mutant BRCA1 tumors (p-value=0.033) when compared to sporadic tumors (FIG. 2). While the BD-L value in primary tumors between sporadic breast cancer patients and BRCA2 mutation carriers was not significant, there is little power in the analysis given the small sample size (n=2). The BD-L values for sporadic primary tumors included a subset with elevated metagene values comparable to those of BRCA1 mutation carriers, which may be indicative of a subpopulation of sporadic tumors with characteristics similar to BRCA1 mutated tumors. The correlation of the BD-L signature with both brain metastases and BRCA1 mutation is consistent with the published literature as BRCA1 mutation carriers are reported to have an increased prevalence of breast cancer brain relapse as compared to noncarriers^{8,24}.

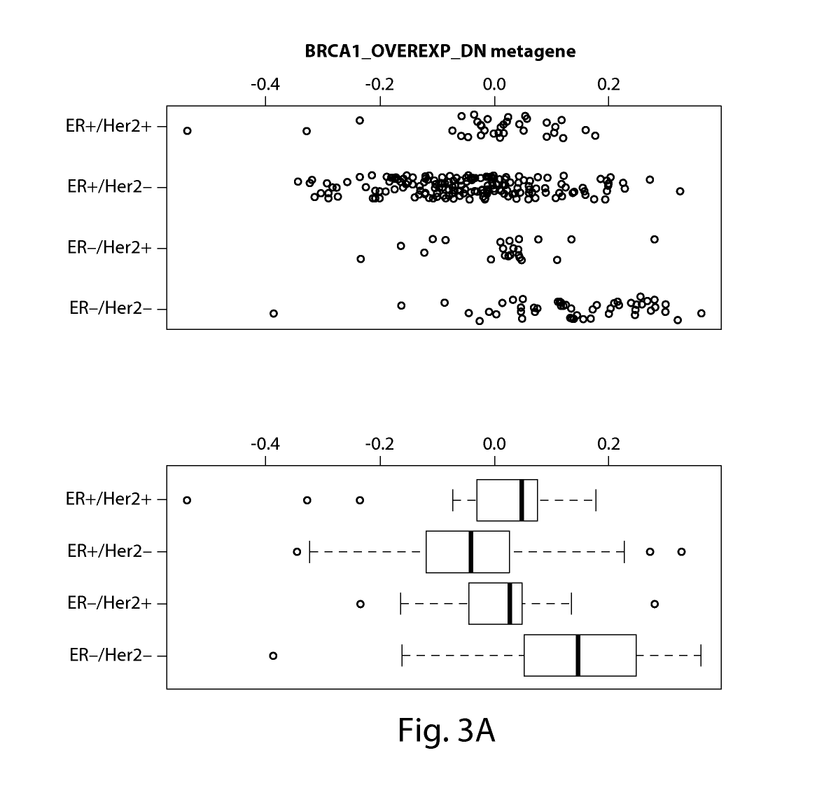

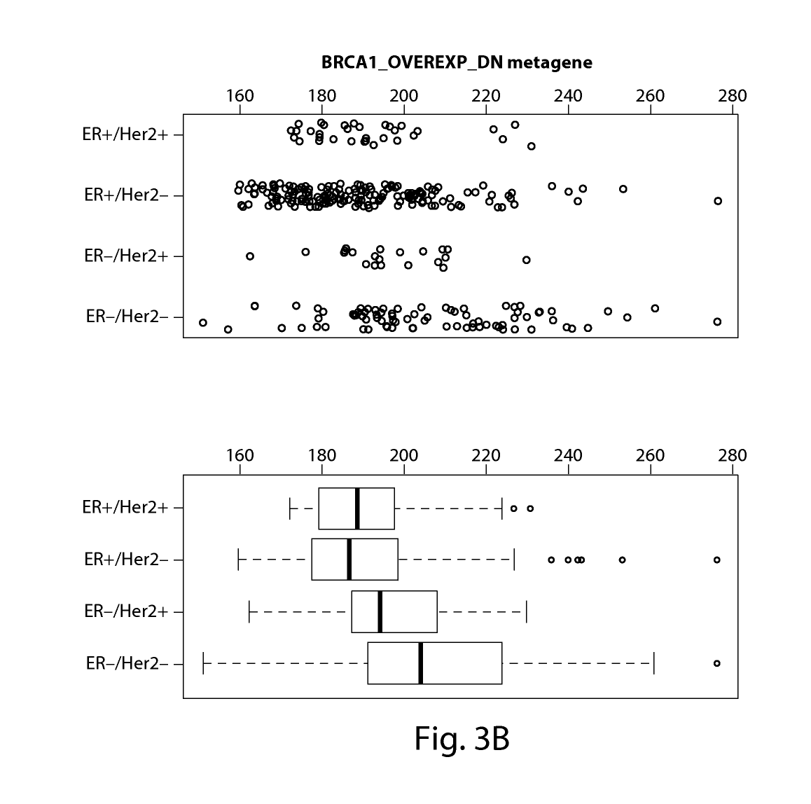

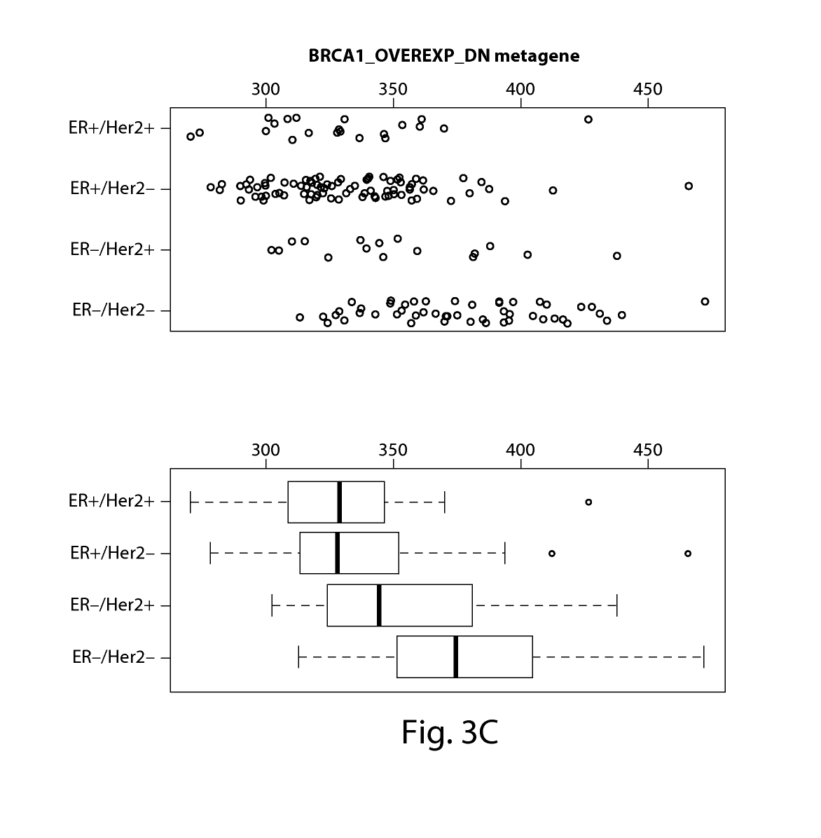

To investigate the correlation of the BD-L metagene with important molecular markers of sporadic breast cancer subtypes, the inventors next evaluated the distribution of BD-L value by HER2 and ER status in the NKI295²⁵, EMC286²⁶/MSK82²⁷, and EMC192²⁸ cohorts of sporadic primary tumors (FIGS. 3A-3C). As BRCA1 mutants represent a subpopulation within the triple negative breast cancer, an expected significantly higher BD-L metagene mean value was observed in ER−/HER− primary tumors when compared to ER+/HER2+(p-value=(A) 4.5×10⁻⁶, (B) 0.0025, (C) 2.8×10⁻⁵) and ER+/HER2− (p-value=(A) 1.1×10⁻¹³, (B) 4×10⁻⁸, (C) 8.7×10⁻¹° subgroups. Although not inconsistent across the cohorts, the trend is observed when comparing ER−/HER− tumors to ER−/HER2+ tumors (p-value=(A) 0.0023, (B) 0.097, (C) 0.05). Despite the significant correlation with a negative ER and/or HER2 receptor expression, it was notable that a small population of tumors with high BD-L value were found within the ER+ and HER2+ subtypes (FIGS. 3A-3C dotplots), indicating that the BD-L phenotype may extend beyond primary tumors of BRCA1 mutation carriers and the sporadic ER−/HER2− subtype. This is especially intriguing for primary ER+ tumors because the brain is not a prevalent metastatic site for the ER+ subtype²⁹. Intrigued by the possibility that the BD-L signature may extend across current breast cancer classifications of receptor expression or mutational status, the inventors next sought to apply the BD-L signature to breast cancer cell lines independent of receptor and mutational status with an aim to identify a phenotype of pharmacological sensitivity.

The inventors hypothesized that the BRCA1 Deficient-Like metagene will identify breast cancers that fall within a spectrum of dysfunction for a BRCA1 functional or regulated pathway. Having demonstrated that BD-L was enriched in BRCA1 mutation carriers, the inventors hypothesized that breast cancer cell lines with elevated BD-L values exhibit increased sensitivity to therapeutic agents that target a dysfunctional BRCA1-associated pathway. Poly(ADP-ribose) polymerase (PARP) inhibitors represent a class of drugs that have demonstrated promise in clinical BRCA1/2 related cancers as single agents^{30,31} and in preclinical studies in combination with certain classes of DNA damaging agents^{32,33}. Because the inventors hypothesize that the BD-L metagene correlates with a spectrum of dysfunction, the inventors chose to induce DNA damage to enhance the effectiveness of the PARP inhibitor. Therefore, a panel of breast cancer cell lines was tested for sensitivity to a combination treatment with the PARP inhibitor olaparib (AZD-2281), an oral PARP inhibitor in clinical use that has shown evidence of crossing the blood/brain barrier³⁴, and the DNA alkylating/methylating agent temozolomide, a clinically utilized chemotherapeutic that can cross the blood/brain barrier and has demonstrated increased efficacy in combination with a PARP inhibitor³⁵⁻³⁸. Using a publicly available gene expression set, the inventors determined BD-L metagene values for 51 well-defined human breast cancer cell lines as described in Neve et al. (Table 7)¹⁵.

TABLE 7
BD-L metagene values for 51 well-defined human breast cancer cell lines.

| | | | | | BRCA1 |
|---|---|---|---|---|---|
| | | | | | Deficient- |
| | | | | | like |
| Cell Line | ER | PR | Her2 | BRCA1 | Metagene |
| HCC1007 | + | − | − | NA | 194.12 |
| HCC1143 | − | − | − | NA | 206.21 |
| MDAMB415 | + | − | − | wt/— | 210.1 |
| SUM44PE | + | − | − | wt/— | 211.62 |
| SUM52PE | + | − | − | wt/— | 218.58 |
| BT20 | − | − | + | wt/— | 221.69 |
| HCC3153 | − | − | − | NA | 223.26 |
| MDAMB157 | − | + | + | wt/— | 223.54 |
| HCC1937 | − | − | − | m/— | 223.65 |
| HCC38 | − | − | − | NA | 224.34 |
| BT483 | + | − | + | wt/wt | 229.94 |
| SUM225CWN | − | + | − | wt/— | 229.98 |
| HCC70 | − | + | − | NA | 230.09 |
| HCC202 | − | − | − | NA | 233.18 |
| HBL100 | − | − | + | NA | 233.63 |
| SUM190PT | − | − | − | wt/— | 235.19 |
| 600MPE | + | − | − | NA | 236.37 |
| ZR7530 | + | − | + | wt/— | 239.09 |
| MDAMB175VII | + | − | + | wt/wt | 239.74 |
| MDAMB453 | − | − | + | wt/— | 240.26 |
| CAMA1 | + | − | − | wt/wt | 240.47 |
| SUM1315MO2 | − | − | − | m/— | 245.27 |
| HS578T | − | − | − | wt/— | 246.63 |
| UACC812 | + | − | − | wt/— | 248.22 |
| ZR751 | + | − | − | wt/wt | 249.87 |
| BT549 | − | − | − | wt/— | 250.23 |
| MCF7 | + | − | − | wt/— | 251.01 |
| HCC1569 | − | − | − | NA | 251.47 |
| SUM149PT | − | − | − | m/— | 254.88 |
| T47D | + | − | − | wt/— | 256.41 |
| MDAMB436 | − | + | − | m/— | 256.73 |
| MDAMB361 | + | − | + | wt/— | 258.05 |
| HCC1187 | − | − | − | NA | 258.64 |
| MDAMB468 | − | − | − | wt/— | 260.85 |
| MDAMB435 | − | − | − | wt/— | 267.2 |
| AU565 | − | − | − | NA | 267.47 |
| BT474 | + | − | − | wt/— | 267.58 |
| HCC1428 | + | − | − | NA | 269.63 |
| MDAMB231 | − | − | − | wt/— | 270.49 |
| ZR75B | + | + | − | NA | 272.72 |
| HCC2185 | − | − | + | NA | 273.16 |
| MDAMB134VI | + | − | − | wt/wt | 273.18 |
| HCC2157 | − | − | + | NA | 282.71 |
| SKBR3 | − | − | − | wt/— | 284.14 |
| MCF12A | − | − | − | NA | 287.31 |
| MCF10A | − | − | + | NA | 288.56 |
| SUM185PE | − | − | − | wt/— | 289.65 |
| LY2 | + | − | − | NA | 293.7 |
| SUM159PT | − | − | − | wt/wt | 294.74 |
| HCC1500 | − | − | − | NA | 303.84 |
| HCC1954 | − | − | − | NA | 304.32 |

The lines were rank-ordered by increasing metagene value, and 12 cell lines were selected as they were predicted to be among either the most resistant or most sensitive to pharmacologic inhibition (Table 1). This panel included the BRCA1-deficient HCC1937 cell line, which the BD-L metagene predicts to exhibit low sensitivity. While this may appear paradoxical, clinical trials have demonstrated that not all BRCA1 mutation carriers are responsive to PARP inhibitors^{30, 31}. Additionally, FIG. 2A demonstrated that although the BD-L metagene is enriched in BRCA1-mutation carriers compared to noncarriers, a subset of BRCA1 mutation carriers have low metagene values. Because the inventors hypothesize the BD-L metagene provides a measure of a BRCA1-associated pathway function rather than a BRCA1 gene mutation or the expression status, the metagene can also account for potential compensatory mechanisms.

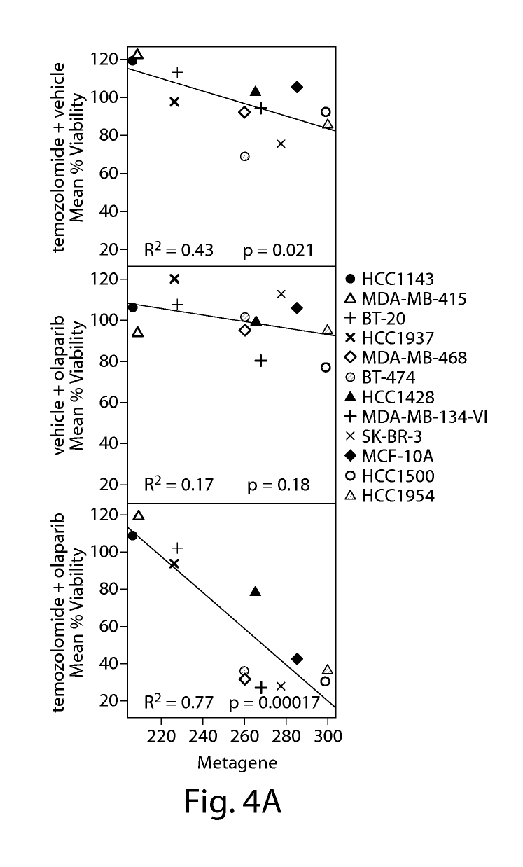

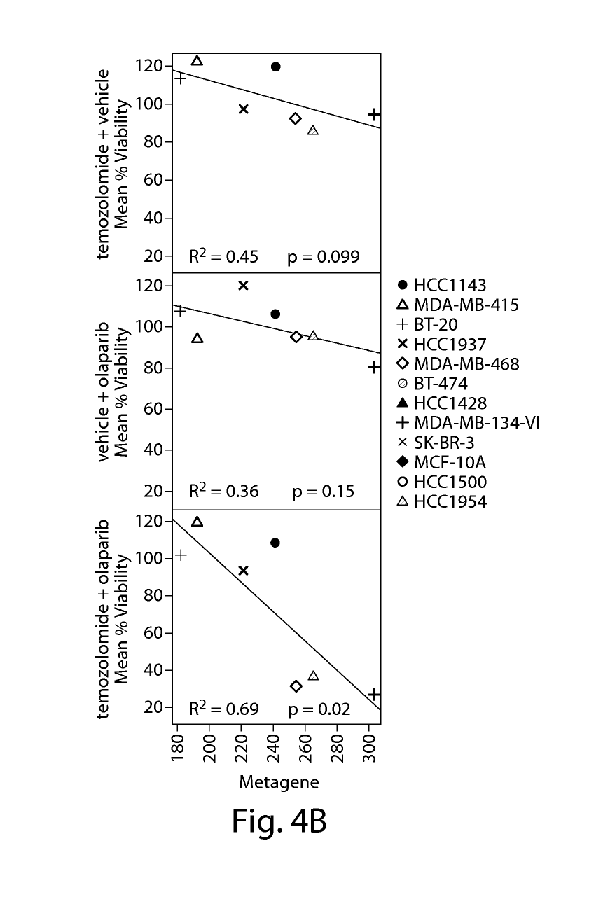

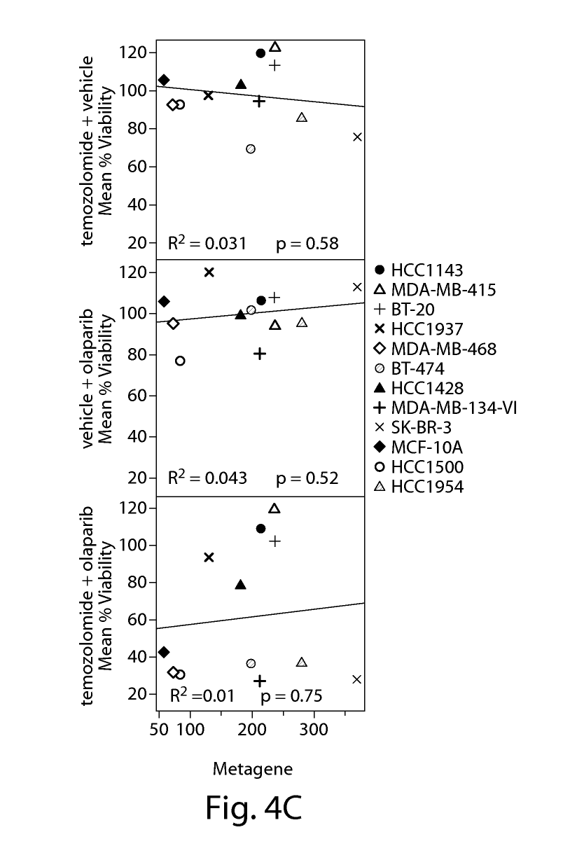

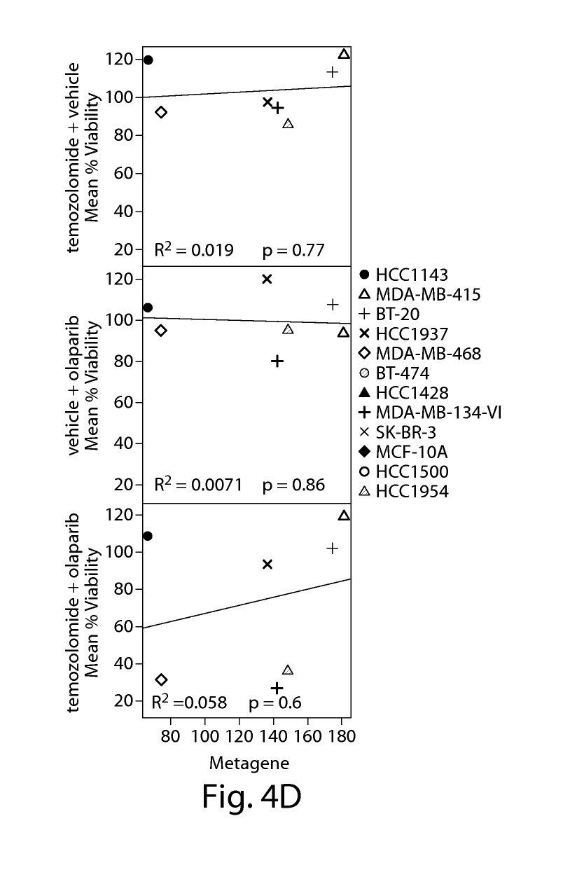

Based upon known mechanisms of temozolomide-specific sensitivity and extensive in vitro pharmacological studies in cell lines³⁹, 100 uM was determined to be a physiologically relevant dose that does not demonstrate significant reduction in viability across the breast cancer cell line panel (FIG. 4A, top panel). Through single treatment and combined treatment with temozolomide, 40 nM olaparib was determined to be a sub-physiological dose that does not demonstrate inhibitory effects alone (FIG. 4A, middle panel), but exerts inhibition when combined with temozolomide. As originally hypothesized, there is a highly significant correlation (R²=0.77; p-value 0.00017) of the BD-L metagene with pharmacological response of cell lines to the combined administration of olaparib and temozolomide (FIG. 4A, lower panel). It is interesting to note that the metagene was able to correctly predict the response of the BRCA1-deficient HCC1937 cell line, indicating the BD-L metagene is a better indicator of pharmacological response than BRCA1 gene status. To further support the correlation with sensitivity, BD-L metagene values were calculated from an independent dataset described in Garnett et al.⁴⁰ that contained gene expression for seven of the tested cell lines. While single administration of either temozolomide or olaparib alone (FIG. 4B, top and middle panel) did not demonstrate a significant reduction in viability, a significant correlation (R²=0.69; p-value 0.02) is observed upon dual administration and supports the inventors' original observations (FIG. 4B, lower panel). Thus, using independent gene expression datasets, the BD-L metagene demonstrated a strong correlation with DNA damage-induced PARP inhibitor sensitivity.

To determine the robustness of BD-L metagene in predicting sensitivity, the inventors evaluated the performance of five published signatures^{23, 41-43} of BRCA1/2 deficiency and/or function in predicting the pharmacologic response of the breast cancer cell line panel using the gene expression data from Neve et al.¹⁵ (FIG. 4C and data not shown) and Garnett et al.⁴⁰ (FIG. 4D and data not shown). In contrast to the BD-L metagene (FIGS. 4A and 4B, bottom panels), all five BRCA1/2 related signatures failed to correlate with pharmacologic response (FIGS. 4C and 4D). The difference in predictive power is potentially due to the approach taken in discovering these signatures. The BD-L metagene was derived from changes in gene expression due to a modest overexpression of BRCA1 within a single cell line. This unbiased approach goes beyond indicating the BRCA1 mutational status or the acute response to a stimulus to provide a measure of BRCA1 pathway function that can include the contribution of BRCA1 and its interacting components. In conclusion, the BD-L metagene provides a measure of BRCA1 pathway function as opposed to indicating BRCA1 mutational status, direct expression levels, or response to an acute stimulus.

In summary, the inventors identified a BRCA1 Deficient-Like metagene that is enriched in HER2+ brain metastases when compared with HER2+ primary tumors, and in an independent dataset confirmed the enrichment of the metagene in brain metastases as compared to bone metastases, lung metastases, and primary breast tumors. Furthermore, the inventors demonstrated that high BD-L metagene value is enriched in, but not limited to, primary tumors of BRCA1 mutation carriers and sporadic ER−/HER2− patients. When the BD-L signature is calculated for a breast cancer cell line panel using gene expression from two independent datasets, the BD-L metagene correlates with pharmacologic response to a combination treatment of olaparib and a temozolomide. Lastly, the inventors demonstrated that the BD-L metagene outperforms extant classifiers of BRCA1/2 status in predicting pharmacological response to the drug combination in the breast cancer cell panel.

Since the clinical administration of PARP inhibitors is still in its infancy, there is a crucial need to both identify patients who will gain benefit from this class of drugs and to develop biomarkers that predict clinical response. Currently, BRCA1/2 status is the prevailing indicator of potential PARP inhibitor sensitivity, although not all BRCA1/2 breast cancers respond and there is preclinical evidence to suggest that PARP inhibitors may hold benefit in cancer populations beyond BRCA1/2 mutation carriers⁴⁴. Herein, the inventors provide evidence that the BD-L metagene is enriched in clinically detectable breast cancer brain metastases and the metagene implicates sporadic breast cancers across the conventional receptor and mutational status classifications that benefit from a PARP inhibitor-based therapy while also identifying triple negative and BRCA1-mutant cancers that prove refractory to treatment.

### REFERENCES

1. Leyland-Jones B. Human epidermal growth factor receptor 2-positive breast cancer and central nervous system metastases. J Clin Oncol. 2009; 27:5278-86.
2. Pienkowski T, Zielinski C C. Trastuzumab treatment in patients with breast cancer and metastatic CNS disease. Ann Oncol. 21:917-24.
3. Weil R J, Palmieri D C, Bronder J L, Stark A M, Steeg P S. Breast cancer metastasis to the central nervous system. Am J Pathol. 2005; 167:913-20.
4. Gabos Z, Sinha R, Hanson J, Chauhan N, Hugh J, Mackey J R, et al. Prognostic significance of human epidermal growth factor receptor positivity for the development of brain metastasis after newly diagnosed breast cancer. J Clin Oncol. 2006; 24:5658-63.
5. Lin N U, Vanderplas A, Hughes M E, Theriault R L, Edge S B, Wong Y N, et al. Clinicopathologic features, patterns of recurrence, and survival among women with triple-negative breast cancer in the National Comprehensive Cancer Network. Cancer.
6. Atchley D P, Albarracin C T, Lopez A, Valero V, Amos C I, Gonzalez-Angulo A M, et al. Clinical and pathologic characteristics of patients with BRCA-positive and BRCA-negative breast cancer. J Clin Oncol. 2008; 26:4282-8.
7. Gonzalez-Angulo A M, Timms K M, Liu S, Chen H, Litton J K, Potter J, et al. Incidence and outcome of BRCA mutations in unselected patients with triple receptor-negative breast cancer. Clin Cancer Res. 17:1082-9.
8. Lee L J, Alexander B, Schnitt S J, Comander A, Gallagher B, Garber J E, et al. Clinical outcome of triple negative breast cancer in BRCA1 mutation carriers and noncarriers. Cancer. 117:3093-100.
9. Olson E M, Abdel-Rasoul M, Maly J, Wu C S, Lin N U, Shapiro C L. Incidence and risk of central nervous system metastases as site of first recurrence in patients with HER2-positive breast cancer treated with adjuvant trastuzumab. Ann Oncol. 24:1526-33.
10. Ma X J, Wang Z, Ryan P D, Isakoff S J, Barmettler A, Fuller A, et al. A two-gene expression ratio predicts clinical outcome in breast cancer patients treated with tamoxifen. Cancer Cell. 2004; 5:607-16.
11. Ma X J, Dahiya S, Richardson E, Erlander M, Sgroi D C. Gene expression profiling of the tumor microenvironment during breast cancer progression. Breast Cancer Res. 2009; 11:R7.
12. Edgar R, Domrachev M, Lash A E. Gene Expression Omnibus: NCBI gene expression and hybridization array data repository. Nucleic Acids Res. 2002; 30:207-10.
13. Rozen S, Skaletsky H. Primer3 on the WWW for general users and for biologist programmers. Methods Mol Biol. 2000; 132:365-86.
14. Holm S. A Simple Sequentially Rejective Multiple Test Procedure. Scandanavian Journal of Statistics. 1979; 6:65-70.
15. Neve R M, Chin K, Fridlyand J, Yeh J, Baehner F L, Fevr T, et al. A collection of breast cancer cell lines for the study of functionally distinct cancer subtypes. Cancer Cell. 2006; 10:515-27.
16. Subramanian A, Tamayo P, Mootha V K, Mukherjee S, Ebert B L, Gillette M A, et al. Gene set enrichment analysis: a knowledge-based approach for interpreting genome-wide expression profiles. Proc Natl Acad Sci USA. 2005; 102:15545-50.
17. Welcsh P L, Lee M K, Gonzalez-Hernandez R M, Black D J, Mahadevappa M, Swisher E M, et al. BRCA1 transcriptionally regulates genes involved in breast tumorigenesis. Proc Natl Acad Sci USA. 2002; 99:7560-5.
18. Armes J E, Trute L, White D, Southey M C, Hammet F, Tesoriero A, et al. Distinct molecular pathogeneses of early-onset breast cancers in BRCA1 and BRCA2 mutation carriers: a population-based study. Cancer Res. 1999; 59:2011-7.
19. Robson M, Rajan P, Rosen P P, Gilewski T, Hirschaut Y, Pressman P, et al. BRCA-associated breast cancer: absence of a characteristic immunophenotype. Cancer Res. 1998; 58:1839-42.
20. Vaziri S A, Krumroy L M, Elson P, Budd G T, Darlington G, Myles J, et al. Breast tumor immunophenotype of BRCA1-mutation carriers is influenced by age at diagnosis. Clin Cancer Res. 2001; 7:1937-45.
21. Plon S E, Eccles D M, Easton D, Foulkes W D, Genuardi M, Greenblatt M S, et al. Sequence variant classification and reporting: recommendations for improving the interpretation of cancer susceptibility genetic test results. Hum Mutat. 2008; 29:1282-91.
22. Zhang X H, Wang Q, Gerald W, Hudis C A, Norton L, Smid M, et al. Latent bone metastasis in breast cancer tied to Src-dependent survival signals. Cancer Cell. 2009; 16:67-78.
23. van't Veer L J, Dai H, van de Vijver M J, He Y D, Hart A A, Mao M, et al. Gene expression profiling predicts clinical outcome of breast cancer. Nature. 2002; 415:530-6.
24. Albiges L, Andre F, Balleyguier C, Gomez-Abuin G, Chompret A, Delaloge S. Spectrum of breast cancer metastasis in BRCA1 mutation carriers: highly increased incidence of brain metastases. Ann Oncol. 2005; 16:1846-7.
25. van de Vijver M J, He Y D, van't Veer L J, Dai H, Hart A A, Voskuil D W, et al. A gene-expression signature as a predictor of survival in breast cancer. N Engl J Med. 2002; 347:1999-2009.
26. Wang Y, Klijn J G, Zhang Y, Sieuwerts A M, Look M P, Yang F, et al. Gene-expression profiles to predict distant metastasis of lymph-node-negative primary breast cancer. Lancet. 2005; 365:671-9.
27. Minn A J, Gupta G P, Siegel P M, Bos P D, Shu W, Giri D D, et al. Genes that mediate breast cancer metastasis to lung. Nature. 2005; 436:518-24.
28. Bos P D, Zhang X H, Nadal C, Shu W, Gomis R R, Nguyen D X, et al. Genes that mediate breast cancer metastasis to the brain. Nature. 2009; 459:1005-9.
29. Clark G M, Sledge G W, Jr., Osborne C K, McGuire W L. Survival from first recurrence: relative importance of prognostic factors in 1,015 breast cancer patients. J Clin Oncol. 1987; 5:55-61.
30. Audeh M W, Carmichael J, Penson R T, Friedlander M, Powell B, Bell-McGuinn K M, et al. Oral poly(ADP-ribose) polymerase inhibitor olaparib in patients with BRCA1 or BRCA2 mutations and recurrent ovarian cancer: a proof-of-concept trial. Lancet. 376:245-51.
31. Tutt A, Robson M, Garber J E, Domchek S M, Audeh M W, Weitzel J N, et al. Oral poly(ADP-ribose) polymerase inhibitor olaparib in patients with BRCA1 or BRCA2 mutations and advanced breast cancer: a proof-of-concept trial. Lancet. 376:235-44.
32. Plummer R. Poly(ADP-ribose) polymerase inhibition: a new direction for BRCA and triple-negative breast cancer? Breast Cancer Res. 13:218.
33. Rouleau M, Patel A, Hendzel M J, Kaufmann S H, Poirier G G. PARP inhibition: PARP1 and beyond. Nat Rev Cancer. 10:293-301.
34. Forster M D, Dedes K J, Sandhu S, Frentzas S, Kristeleit R, Ashworth A, et al. Treatment with olaparib in a patient with PTEN-deficient endometrioid endometrial cancer. Nat Rev Clin Oncol. 8:302-6.
35. Donawho C K, Luo Y, Luo Y, Penning T D, Bauch J L, Bouska J J, et al. ABT-888, an orally active poly(ADP-ribose) polymerase inhibitor that potentiates DNA-damaging agents in preclinical tumor models. Clin Cancer Res. 2007; 13:2728-37.
36. Liu X, Shi Y, Guan R, Donawho C, Luo Y, Palma J, et al. Potentiation of temozolomide cytotoxicity by poly(ADP)ribose polymerase inhibitor ABT-888 requires a conversion of single-stranded DNA damages to double-stranded DNA breaks. Mol Cancer Res. 2008; 6:1621-9.
37. Miknyoczki S J, Jones-Bolin S, Pritchard S, Hunter K, Zhao H, Wan W, et al. Chemopotentiation of temozolomide, irinotecan, and cisplatin activity by CEP-6800, a poly(ADP-ribose) polymerase inhibitor. Mol Cancer Ther. 2003; 2:371-82.
38. Plummer R, Jones C, Middleton M, Wilson R, Evans J, Olsen A, et al. Phase I study of the poly(ADP-ribose) polymerase inhibitor, AG014699, in combination with temozolomide in patients with advanced solid tumors. Clin Cancer Res. 2008; 14:7917-23.
39. Fu D, Calvo J A, Samson L D. Balancing repair and tolerance of DNA damage caused by alkylating agents. Nat Rev Cancer. 12:104-20.
40. Garnett M J, Edelman E J, Heidorn S J, Greenman C D, Dastur A, Lau K W, et al. Systematic identification of genomic markers of drug sensitivity in cancer cells. Nature. 483:570-5.
41. Konstantinopoulos P A, Spentzos D, Karlan B Y, Taniguchi T, Fountzilas E, Francoeur N, et al. Gene expression profile of BRCAness that correlates with responsiveness to chemotherapy and with outcome in patients with epithelial ovarian cancer. J Clin Oncol. 28:3555-61.
42. Kote-Jarai Z, Matthews L, Osorio A, Shanley S, Giddings I, Moreews F, et al. Accurate prediction of BRCA1 and BRCA2 heterozygous genotype using expression profiling after induced DNA damage. Clin Cancer Res. 2006; 12:3896-901.
43. Kote-Jarai Z, Williams R D, Cattini N, Copeland M, Giddings I, Wooster R, et al. Gene expression profiling after radiation-induced DNA damage is strongly predictive of BRCA1 mutation carrier status. Clin Cancer Res. 2004; 10:958-63.
44. Gelmon K A, Tischkowitz M, Mackay H, Swenerton K, Robidoux A, Tonkin K, et al. Olaparib in patients with recurrent high-grade serous or poorly differentiated ovarian carcinoma or triple-negative breast cancer: a phase 2, multicentre, open-label, non-randomised study. Lancet Oncol.12:852-61.

1. A method of treating cancer in a subject, the method comprising:
(a) assaying a biological sample comprising a cancer cell obtained from a subject having cancer or suspected of having cancer for the expression of a combination of at least the following markers: EZR, SOX4, EMG1, NARS, SNRNP70, CLTA, EIF3B, EIF3E, UBAP2L, SF3A3, HK1, CKAP4, NOLC1, HCFC1, PPM1F, BMS1, AIMP2, POLG, MCM2, U2AF1, MOGS, SKIV2L, CCND1, MLEC, RARS, SLC7A5, MANF, RAE1, CHD4, BYSL, GTF3C2, TRAP1, KIAA0114, CDC25B, SF3B2, SMARCA4, DDX11, LMNB2, DHCR24, SAFB, CTPS, DUSP1, DDX10, CDC123, NDRG1, EIF2B2, FAM38A, SEC24C, RABEPK, FGFR3, SSRP1, and SLC1A3;
(b) normalizing the expression data for each of the markers to a control, and
(c) administering to the subject a therapeutically effective amount of a poly ADP ribose polymerase (PARP) inhibitor and a DNA damage-inducing chemotherapeutic agent when expression of the combination of markers is increased compared to a reference value and not administering a PARP inhibitor and a DNA damage-inducing chemotherapeutic agent when the expression of the combination of markers is not changed or decreased compared to a reference value.

2. (canceled)

3. The method of claim 1 or 2, further comprising assaying the biological sample for at least one additional marker selected from the group consisting of: GCSH, BOP1, XRCC5, CCDC85B, H1FX, FDFT1, DNAJC7, PRKACA, MAP2K5, SNRPA1, RELA, RXRA, KDMSC, PWP2, NME3, FARSA, DNTTIP2, MRPS12, SCRIB, PPAT, CDC37, SLC29A1, SFRS11, SMAD6, EIF4H, VDAC1, NMT1, POLRMT, POLD1, CHAF1A, TAF1C, TCOF1, ALG3, CAD, RASSF7, MTHFD1, CCNE1, HSF1, PSMD1, AGPAT2, PHKG2, PKN1, GAL, SCAP, EIF4G1, DDT, PMPCA, RRP1B, FKBP4, NUP214, ATIC, FASN, AP3D1, RPIA, NSDHL, BRD2, PDCD11, SON, HSP90B1, and ANXA11.

4. The method of claim 1 or 2, wherein the control is the expression level of the total set of markers assayed.

5. The method of claim 1 or 2, further comprising a step of calculating a metagene value to represent the normalized expression of the combination of markers.

6. The method of claim 1 or 2, wherein the step of calculating the metagene comprises linear combination of the expression values of each marker in the combination of markers.

7. The method of claim 6, wherein the linear combination has a coefficient of 1 for each marker.

8. The method of claim 1 or 2, wherein the biological sample comprises a tumor sample.

9. The method of claim 3, wherein the biological sample is assayed for expression of all of the markers SFRS11, EZR, PKN1, SOX4, EMG1, RELA, AIMP2, NARS, HSP90B1, SNRP70, NME3, CLTA, CAD, EIF3B, EIF3E, UBAP2L, SF3A3, HK1, TCOF1, CKAP4, CCNE1, NOLC1, HCFC1, PPM1F, BMS1L, RASSF7, FDFT1, FARSLA, POLG, MCM2, VDAC1, U2AF1, MOGS, ATIC, CHAF1A, SKIV2L, GCSH, GAL, CCDC85B, CCND1, MLEC, RARS, WBSCR1, FKBP4, SLC7A5, MTHFD1, NSDHL, PWP2H, FASN, MANF, EIF4G1, JARID1C, SCRIB, SCAP, PSMD1, RAE1, CHD4, SLC29A1, BYSL, GTF3C2, TRAP1, PRKACA, KIAA0179, KIAA0114, POLD1, DNTTIP2, BOP1, CDC25B, MRPS12, SF3B2, NUP214, PHKG2, AGPAT2, SMARCA4, PPAT, SNRPA1, DDX11, RPIA, ALG3, LMNB2, TAF1C, DDT, DHCR24, SAFB, CDC37, H1FX, AP3D1, DNAJC7, HSF1, CTPS, DUSP1, DDX10, PDCD11, CDC123, RXRA, NDRG1, EIF2B2, ANXA11, FAM38A, XRCC5, SEC24C, PMPCA, POLRMT, RABEPK, SMAD6, NMT1, SON, MAP2K5, SLC1A3, FGFR3, SSRP1, and BRD2.

10.-12. (canceled)

13. The method of claim 1, wherein the cancer is breast cancer.

14.-15. (canceled)

16. A method for selecting a PARP inhibitor/DNA damaging chemotherapeutic-sensitive or PARP inhibitor/DNA damaging chemotherapeutic-insensitive subject having cancer, the method comprising:
(a) assaying a biological sample comprising a cancer cell obtained from a subject having cancer or suspected of having cancer for the expression of a combination of at least the following markers: EZR, SOX4, EMG1, NARS, SNRNP70, CLTA, EIF3B, EIF3E, UBAP2L, SF3A3, HK1, CKAP4, NOLC1, HCFC1, PPM1F, BMS1, AIMP2, POLG, MCM2, U2AF1, MOGS, SKIV2L, CCND1, MLEC, RARS, SLC7A5, MANF, RAE1, CHD4, BYSL, GTF3C2, TRAP1, KIAA0114, CDC25B, SF3B2, SMARCA4, DDX11, LMNB2, DHCR24, SAFB, CTPS, DUSP1, DDX10, CDC123, NDRG1, EIF2B2, FAM38A, SEC24C, RABEPK, FGFR3, SSRP1, and SLC1A3;
(b) normalizing the expression data for each of the markers to a control, and
(c) selecting the subject as a PARP inhibitor/DNA damaging chemotherapeutic-sensitive subject when expression of the combination of markers is increased compared to a reference value, and
selecting the subject as a PARP inhibitor/DNA damaging chemotherapeutic-insensitive subject when expression of the combination of markers is not changed or decreased compared to the reference value.

17. The method of claim 16, further comprising assaying the biological sample for at least one additional marker selected from the group consisting of: GCSH, BOP1, XRCC5, CCDC85B, H1FX, FDFT1, DNAJC7, PRKACA, MAP2K5, SNRPA1, RELA, RXRA, KDM5C, PWP2, NME3, FARSA, DNTTIP2, MRPS12, SCRIB, PPAT, CDC37, SLC29A1, SFRS11, SMAD6, EIF4H, VDAC1, NMT1, POLRMT, POLD1, CHAF1A, TAF1C, TCOF1, ALG3, CAD, RASSF7, MTHFD1, CCNE1, HSF1, PSMD1, AGPAT2, PHKG2, PKN1, GAL, SCAP, EIF4G1, DDT, PMPCA, RRP1B, FKBP4, NUP214, ATIC, FASN, AP3D1, RPIA, NSDHL, BRD2, PDCD11, SON, HSP90B1, and ANXA11.

18. The method of claim 17, wherein the biological sample is assayed for expression of all of the markers SFRS11, EZR, PKN1, SOX4, EMG1, RELA, AIMP2, NARS, HSP90B1, SNRP70, NME3, CLTA, CAD, EIF3B, EIF3E, UBAP2L, SF3A3, HK1, TCOF1, CKAP4, CCNE1, NOLC1, HCFC1, PPM1F, BMS1L, RASSF7, FDFT1, FARSLA, POLG, MCM2, VDAC1, U2AF1, MOGS, ATIC, CHAF1A, SKIV2L, GCSH, GAL, CCDC85B, CCND1, MLEC, RARS, WBSCR1, FKBP4, SLC7A5, MTHFD1, NSDHL, PWP2H, FASN, MANF, EIF4G1, JARID1C, SCRIB, SCAP, PSMD1, RAE1, CHD4, SLC29A1, BYSL, GTF3C2, TRAP1, PRKACA, KIAA0179, KIAA0114, POLD1, DNTTIP2, BOP1, CDC25B, MRPS12, SF3B2, NUP214, PHKG2, AGPAT2, SMARCA4, PPAT, SNRPA1, DDX11, RPIA, ALG3, LMNB2, TAF1C, DDT, DHCR24, SAFB, CDC37, H1FX, AP3D1, DNAJC7, HSF1, CTPS, DUSP1, DDX10, PDCD11, CDC123, RXRA, NDRG1, EIF2B2, ANXA11, FAM38A, XRCC5, SEC24C, PMPCA, POLRMT, RABEPK, SMAD6, NMT1, SON, MAP2K5, SLC1A3, FGFR3, SSRP1, and BRD2.

19. The method of claim 16, wherein the control is the expression level of the total set of markers assayed.

20. The method of claim 16, further comprising a step of calculating a metagene value to represent the normalized expression of the combination of markers.

21. The method of claim 20, wherein the step of calculating the metagene comprises linear combination of the expression values of each marker in the combination of markers.

22. The method of claim 21, wherein the linear combination has a coefficient of 1 for each marker.

23. The method of claim 16, wherein the biological sample comprises a tumor sample.

24.-26. (canceled)

27. The method of claim 16, wherein the cancer is breast cancer.

28.-46. (canceled)

47. A composition comprising an array consisting essentially of probes directed to the following genes: EZR, SOX4, EMG1, NARS, SNRNP70, CLTA, EIF3B, EIF3E, UBAP2L, SF3A3, HK1, CKAP4, NOLC1, HCFC1, PPM1F, BMS1, AIMP2, POLG, MCM2, U2AF1, MOGS, SKIV2L, CCND1, MLEC, RARS, SLC7A5, MANF, RAE1, CHD4, BYSL, GTF3C2, TRAP1, KIAA0114, CDC25B, SF3B2, SMARCA4, DDX11, LMNB2, DHCR24, SAFB, CTPS, DUSP1, DDX10, CDC123, NDRG1, EIF2B2, FAM38A, SEC24C, RABEPK, FGFR3, SSRP1, and SLC1A3; and attached to a solid support.

48. The composition of claim 47, wherein the probes are cDNA probes.

49.-65. (canceled)
